(19) United States
(12) Patent Application Publication
(10) Pub. No.: US 2024/0293401 A1
(43) Pub. Date: Sep. 5, 2024

(54) COMBINATION THERAPY WITH AN LXR AGONIST AND AN INHIBITOR COMPOUND

(71) Applicant: Asteroid Therapeutics, Indianapolis, IN (US)

(72) Inventors: Keith Lenden, San Carlos, CA (US); Steve Paul, San Carlos, CA (US); Francesco G. Salituro, Marlborough, MA (US)

(21) Appl. No.: 18/581,623

(22) Filed: Feb. 20, 2024

Related U.S. Application Data
(60) Provisional application No. 63/485,588, filed on Feb. 17, 2023.

(51) Int. Cl. A61K 31/497; A61K 31/18; A61K 31/192; A61K 31/195; A61K 31/341; A61K 31/404

(52) U.S. Cl. CPC A61K 31/497; A61K 31/18; A61K 31/192; A61K 31/195; A61K 31/341; A61K 31/404

(57) ABSTRACT

The invention describes novel methods of treating patients with LXR- or RXR-mediated disease using an Agonist selected from an LXR Agonist or an RXR Agonist in combination with an Inhibitor compound, wherein the Inhibitor is given to ameliorate one or more side effects of the selected Agonist. Examples of the Inhibitor include an LXR Antagonist, an LXR Inverse Agonist, and, a combination thereof.

### CROSS REFERENCE TO RELATED APPLICATIONS

This application is entitled to priority pursuant to 35 U.S.C. § 119(e) to U.S. provisional patent application No. 63/485,588, filed on 17 Feb. 2023. The contents of which are incorporated herein by reference in their entirety.

### BACKGROUND OF THE INVENTION

The liver X receptor (LXR), a nuclear receptor transcription factor, is known to be an important regulator of lipid homeostasis. 22(R)-hydroxycholesterol, and 24(S)-hydroxycholesterol, both LXR agonists, have been shown to suppress the proliferation of glioblastoma, prostate cancer, and breast cancer cells as well as to delay progression of prostate cancer to androgen-independent or a hormone-refractory state. So, the agonism of LXR serves as a potential therapeutic treatment for lipogenic cancers, leading to the reduction in tumor viability, cell growth, cell size and migration.

The LXR receptor is also known to be involved in numerous other disease-related pathways. Activation of the LXR receptors has been suggested to treat diseases including Alzheimer's, atherosclerosis, coronary artery disease, type II diabetes, skin aging, etc. (see, for example, US Patent Publication No. 20220363662A1 and U.S. Pat. No. 9,751,869B2). So, the agonism of the LXR also serves as a potential therapeutic treatment for these diseases.

While LXR agonists have shown promise in both animal and human studies, various side effects have been observed. For example, the Alzheimer's Drug Discovery Foundation reports that LXR agonists are potential treatments for Alzheimer's disease and atherosclerosis, but LXR agonists may increase the risk of metabolic diseases because they are known to increase triglyceride levels. Maczewsky et al. report that LXR agonists, specifically T0901317 and GW3965, can “interfere with mitochondrial metabolism and metabolism-independent processes in β-cells (see Endocrine 2020, 68, 526-535).

Since it is believed that activation of LXRα is the primary source of the side effects, companies have attempted to develop LXR agonists that primarily activate LXRβ. However, a recently developed LXR agonist that is primarily an LXRβ agonist (20% LXRα agonist) still showed substantial side effects. For example, Kirchgessner et al. report that BMS-852929, which was being developed for treatment of coronary artery disease, “showed increased plasma and hepatic [triglycerides], plasma LDL-C, apoB, apoE, and [cholesterol ester transfer protein] and decreased circulating neutrophils” (see Cell Metabolism 2016, 24, 223-233). Fessler, M. B., reports that “systemic LXR activation is complicated by hepatic steatosis and hypertriglyceridemia” (see Pharmacol. Ther. 2018, January, 181, 1-12).

The RXR agonist Targretin (bexarotene) is a clinically used antitumoral agent that exerts its action through binding to and activation of the retinoid-X-receptor (RXR) (see Endocrinology 2009, May, 150(5), 2368-2375). In recent years, additional evidence has demonstrated potential therapeutic application of RXR agonist(s) for neurodegenerative diseases (including Alzheimer's disease), immuno-oncology, and inflammation. The most frequent side-effect of bexarotene administration is an increase in plasma triglycerides, which is reported to be partially attributable to LXR target gene activation in the liver.

In view of the documented benefits of LXR agonists and RXR agonists, there is a need in the art to improve the side effect profiles of these agonists to clinically develop them. Thus, it would be beneficial to design a method of using LXR agonists and RXR agonists while attenuating or eliminating their inherent side effects.

### SUMMARY OF THE INVENTION

In an aspect, there are described methods of treating an LXR-mediated or RXR-mediated disease in a patient, comprising administering:
(a) a therapeutically effective amount of an Agonist selected from an LXR Agonist and a RXR Agonist; and,
(b) a therapeutically effective amount of an Inhibitor compound that ameliorates one or more side effects associated with the use of the Agonist.

In another aspect, there are described methods of combination therapy, comprising:
(a) administering, to a patient in need thereof, a therapeutically effective amount of an Agonist selected from an LXR Agonist and a RXR Agonist; and,
(b) administering a therapeutically effective amount of an Inhibitor compound that ameliorates one or more side effects associated with the use of the Agonist; wherein the patient suffers from a disease that is ameliorated by activation of the selected Agonist receptors.

In another aspect, there are described Inhibitor compounds that are:
i) an LXR Antagonist,
ii) an LXR Inverse Agonist, and,
iii) a combination thereof.

In another aspect, there are described pharmaceutical compositions, comprising an Agonist and an LXR Inhibitor compound and optionally a pharmaceutically acceptable carrier.

In another aspect, there are described compounds and/or compositions for use in medical therapy.

In another aspect, there are described the use of compounds for the manufacture of a medicament for the treatment of an indication described herein.

These and other aspects, which will become apparent during the following detailed description, have been achieved by the inventors' discovery that the side effects of an Agonist (RXR or LXR) can be attenuated by an Inhibitor compound that ameliorates one or more side effects associated with the use of the Agonist.

### DETAILED DESCRIPTION OF THE INVENTION

Exemplary aspects of the invention are described herein. Although the following detailed description contains many specifics for purposes of illustration, a person of ordinary skill in the art will appreciate that variations and alterations to the following details are within the scope of the invention. Accordingly, the following aspects of the invention are set forth without any loss of generality to, and without imposing limitations upon, the claimed invention.

### Definitions

The examples provided in the definitions present in this application are non-inclusive unless otherwise stated. They include but are not limited to the recited examples.

When introducing elements of the present disclosure or an aspect thereof, the articles “a”, “an”, “the” and “said” are intended to mean that there are one or more of the elements. The terms “comprising”, “including” and “having” are intended to be inclusive and mean that there may be additional elements other than the listed elements.

The term “and/or” when used in a list of two or more items, means that any one of the listed items can be employed by itself or in combination with any one or more of the listed items. For example, the expression “A and/or B” is intended to mean either or both of A and B, i.e. A alone, B alone or A and B in combination. The expression “A, B and/or C” is intended to mean A alone, B alone, C alone, A and B in combination, A and C in combination, B and C in combination or A, B, and C in combination.

“Adverse events” see “side effects.”

“Agonist” refers to an agonist selected from an LXR Agonist and a RXR Agonist

“Alleviates” refers to the Inhibitor reducing or preventing the occurrence of a side effect of the Agonist.

“Blood brain barrier” (BBB) refers to the barrier that prevents blood solutes (e.g., certain drugs) from non-selectively crossing into the extracellular fluid of the central nervous system. Compounds that do not cross the BBB are typically classified as being peripherally restricted.

“Controlled release” is defined as a prolonged release pattern of one or more drugs, such that the drugs are released over a period of time. A controlled release formation is a formulation with release kinetics that result in measurable serum levels of the drug over a period longer than what would be possible following intravenous injection or following administration of an immediate release oral dosage form. Controlled release, slow release, sustained release, extended release, prolonged release, and delayed release have the same definitions for the present invention.

“Effective amount” or “pharmaceutically effective amount” are used interchangeably and are defined to mean the amount or quantity of steroidal compound, which is sufficient to elicit an appreciable biological response when administered to a patient. It will be appreciated that the precise therapeutic dose will depend on the age and condition of the patient and the nature of the condition to be treated and will be at the ultimate discretion of the attendant physician.

“Lipogenic cancers” include brain (GBM), breast, lung, melanoma, hepatocellular carcinoma (HCC), colon, prostate, and ovarian cancers.

“Liver tissue specificity” refers to the liver exposure of the Inhibitor compound being higher than the plasma or other organ tissue exposure. For example, the Inhibitor compound loads and sequesters disproportionally in the liver versus the plasma (or other organ tissue). In some aspects, an Inhibitor compound having liver tissue specificity has a higher molarity in the liver than in the plasma (or other organ tissue) measured after administration. Examples of liver/plasma ratios (molar ratios) include >1, 5, 10, 15, 20, 25, 30, 35, 40, 45, 50, 100, 150, 200, 250, 300, 350, 400, 450, 500 or more. These ratios are determined at a certain time (or times) post Inhibitor compound administration. For example, the liver/plasma ratio can be measured 1, 2, 3, 4, 5, 6, or more hours after administration.

“LXR” is the Liver X receptor.

“LXR Agonist” means a molecule that functions as an agonist of the Liver X receptor at a pharmacological concentration thereby increasing the activity or signaling of this receptor.

“Inhibitor” means a molecule that functions as an antagonist or inverse agonist of the Liver X receptor. An antagonist typically blocks the function of an agonist. An inverse agonist typically decreases the activity or signaling of the receptor irrespective of the presence of an agonist.

“Mammal” includes human, primate, bovine, porcine, canine, feline, and rodent (e.g., mice and rats).

“Maximum tolerated dose” means the highest dose of a drug or therapeutic that can be taken by patients without the patients' experiencing intolerable side effects. The maximum tolerated dose is typically determined empirically in clinical trials.

“Peripheral restriction” or “peripherally restricted” refers to compounds that are either unable to cross the BBB or only able to cross in such a low concentration that no therapeutic effect (or adverse effect) is observed in the brain.

“Parenteral administration” and “administered parenterally” are art-recognized and refer to modes of administration other than enteral and topical administration, usually by injection, and includes, without limitation, intravenous, intramuscular, intraarterial, intrathecal, intracapsular, intraorbital, intracardiac, intradermal, intraperitoneal, transtracheal, subcutaneous, subcuticular, intra-articular, subcapsular, subarachnoid, intraspinal, and intrasternal injection and infusion.

“Patient,” “subject” or “host” to be treated by the subject method may mean either a human or non-human mammal.

“Pharmaceutically acceptable salts” refer to derivatives of the disclosed compounds wherein the parent compound is modified by making acid or base salts thereof.

“Therapeutically effective amount” includes an amount of a compound of the invention that is effective when administered alone or in combination to an indication listed herein. “Therapeutically effective amount” also includes an amount of the combination of compounds claimed that is effective to treat the desired indication. The combination of compounds can be a synergistic combination. Synergy, as described, for example, by Chou and Talalay, Adv. Enzyme Regul. 1984, 22:27-55, occurs when the effect of the compounds when administered in combination is greater than the additive effect of the compounds when administered alone as a single agent. In general, a synergistic effect is most clearly demonstrated at sub-optimal concentrations of the compounds. Synergy can be in terms of lower cytotoxicity, increased effect, or some other beneficial effect of the combination compared with the individual components.

“Reducing” refers to the side effect being measurably less severe that in the absence of the Inhibitor.

“RXR” is the Retinoid X receptor.

“RXR Agonist” means a molecule that functions as an agonist of the Retinoid X receptor at a pharmacological concentration thereby increasing the activity or signaling of this receptor.

“Side effects” and “adverse events” can be used interchangeably and refer to undesirable secondary effects that result from the administration of an LXR agonist. These are further defined below.

“Treating” or “treatment” covers the treatment of a disease-state in a mammal, and includes: (a) preventing the disease-state from occurring in a mammal, in particular, when such mammal is predisposed to the disease-state but has not yet been diagnosed as having it; (b) inhibiting the disease-state, e.g., arresting its development; and/or (c) relieving the disease-state, e.g., causing regression of the disease state until a desired endpoint is reached. Treating also includes the amelioration of a symptom of a disease (e.g., lessen the pain or discomfort), wherein such amelioration may or may not be directly affecting the disease (e.g., cause, transmission, expression, etc.).

### Methods

In an aspect, the invention involves methods, comprising administering to a patient in need thereof:
(a) a therapeutically effective amount of an Agonist selected from an LXR Agonist and an RXR Agonist; and,
(b) a therapeutically effective amount of an Inhibitor compound that ameliorates one or more side effects associated with the use of the Agonist.

In another aspect, the method is one of treating an LXR-mediated disease in a patient having an LXR-mediated disease.

In another aspect, the method is one of treating an RXR-mediated disease in a patient having an RXR-mediated disease.

In another aspect, the patient is susceptible to LXR agonist side effects.

In another aspect, the patient is susceptible to RXR agonist side effects.

In another aspect, the method involves combination therapy and a patient who suffers from a disease that is ameliorated by activation of LXR receptors.

In another aspect, the method involves combination therapy and a patient who suffers from a disease that is ameliorated by activation of RXR receptors.

In another aspect, the present invention involves a method of combination therapy, comprising:
(a) administering, to a patient in need thereof, a therapeutically effective amount of an Agonist selected from an LXR Agonist and an RXR Agonist; and,
(b) administering a therapeutically effective amount of an Inhibitor compound that ameliorates one or more side effects associated with the use of the Agonist;
wherein the patient suffers from a disease that is ameliorated by activation of the selected Agonist receptors.

In another aspect, the method is one of treating an LXR-mediated disease in a patient having an LXR-mediated disease.

In another aspect, the method is one of treating an RXR-mediated disease in a patient having an RXR-mediated disease.

In another aspect, the patient is susceptible to LXR agonist side effects.

In another aspect, the patient is susceptible to RXR agonist side effects.

In another aspect, the method involves combination therapy and a patient who suffers from a disease that is ameliorated by activation of LXR receptors.

In another aspect, the method involves combination therapy and a patient who suffers from a disease that is ameliorated by activation of RXR receptors.

In another aspect, the LXR Agonist meets the following criteria:
(a) LXRb RLB Ki <100 nM;
(b) LXR Agonist Reporter assay EC50 <300 nM; and,
(c) Free drug exposure Cmax in mouse liver >in vitro EC50.

LXRb RLB can be assayed as described in Br J Pharmacol 2011, April, 162(7), 1553-63 and US 2023/0340011, published Oct. 26, 2023.

LXR Reporter assay is described in ACS Pharmacol Transl Sci. 2021 Feb. 12, 4(1), 143-154.

Free drug exposure Cmax in mouse liver can be determined as described in J Pharm Sci. 2021, March, 110(3), 1401-1411.

In another aspect, the RXR Agonist meets the following criteria:
(a) RXR Ki <100 nM;
(b) RXR Reporter assay EC50 <200 nM (in M2H luciferase-based RXR-RXR homodimerization system); and,
(c) Free drug exposure Cmax in mouse liver >in vitro EC50.

RXR Agonism can be assayed as described by Warda, A. et al. in Cells 2023, November, 12(21), 2575.

In another aspect, the Agonist is selected from:

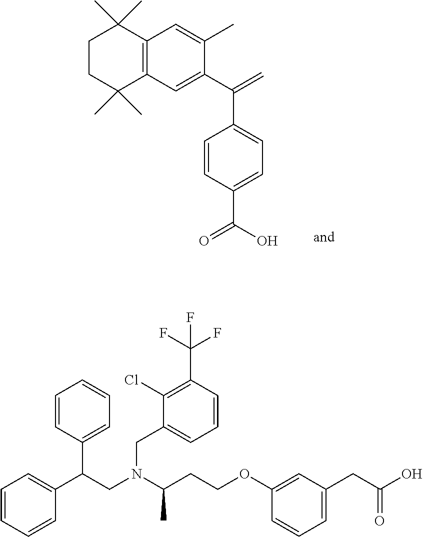 [SMILES: C=C(c1ccc(C(=O)O)cc1)c1cc2c(cc1C)C(C)(C)CCC2(C)C ; C[C@H](CCOc1cccc(CC(=O)O)c1)N(Cc1cccc(C(F)(F)F)c1Cl)CC(c1ccccc1)c1ccccc1]

or a pharmaceutically acceptable salt thereof.

### LXR-Mediated Diseases

In another aspect, the LXR-mediated disease is selected from age and UV exposure-dependent skin wrinkling, asthma, atherosclerosis, atopic dermatitis, cancer (e.g., endometrial cancer, glioblastoma (GBM), lung cancer (e.g., small cell and non-small cell), lymphoma, and solid malignancies), cardiac steatosis, coronary artery disease, dementia with Lewy bodies, dyslipidemia, familial hypercholesterolemia, fronto-temporal dementias, hepatitis C virus infection or its complications, hypercholesterolemia in nephrotic syndrome, hyperglycemia, hyperlipidemia, hypertension, inflammation, inflammatory bowel disease, insulin resistance, liver fibrosis, liver inflammation, macular degeneration, metabolic syndrome, multiple sclerosis, neurodegenerative diseases (e.g., Alzheimer's disease, amyotrophic lateral sclerosis, Huntington's disease, and Parkinson's disease), non-alcoholic fatty liver disease (NAFLD), non-alcoholic steatohepatitis (NASH), obesity, osteoporosis, osteoarthritis, peripheral neuropathy, psoriasis, rheumatoid arthritis, stroke, type II diabetes, viral myocarditis, and unwanted side-effects of long-term glucocorticoid treatment in diseases such as rheumatoid arthritis.

In another aspect, the LXR indication is a CNS indication.

Examples of CNS indications include dementia with Lewy bodies, fronto-temporal dementias, glioblastoma (GBM), and neurodegenerative diseases (e.g., Alzheimer's disease, amyotrophic lateral sclerosis, Huntington's disease, and Parkinson's disease).

In another aspect, the LXR indication is a liver indication.

Examples of liver indications include non-alcoholic fatty liver disease (NAFLD), non-alcoholic steatohepatitis (NASH), cardiac steatosis, liver fibrosis, liver inflammation, type II diabetes,

In another aspect, the LXR indication is a non-CNS, non-liver indication.

Examples of peripheral, non-liver indications include age and UV exposure-dependent skin wrinkling, asthma, atopic dermatitis, atherosclerosis, dyslipidemia, coronary artery disease, familial hypercholesterolemia, hepatitis C virus infection or its complications, hypercholesterolemia in nephrotic syndrome, hyperglycemia, hyperlipidemia, hypertension, inflammation, inflammatory bowel disease, insulin resistance, macular degeneration, metabolic syndrome, multiple sclerosis, obesity, osteoporosis, osteoarthritis, peripheral neuropathy, psoriasis, rheumatoid arthritis, stroke, viral myocarditis, and unwanted side-effects of long-term glucocorticoid treatment in diseases such as rheumatoid arthritis.

In another aspect, the RXR-mediated disease is selected from cutaneous T-cell lymphoma (CTCL), neurodegenerative diseases (e.g., Alzheimer's disease, amyotrophic lateral sclerosis, Huntington's disease, and Parkinson's disease), immune-oncology (e.g., HER2⁺ breast cancer and Kras-Driven lung cancer), and an inflammatory disease (e.g., inflammation, inflammatory bowel disease, liver inflammation, and rheumatoid arthritis).

In another aspect, the RXR indication is a CNS indication.

Examples of CNS indications include neurodegenerative diseases (e.g., Alzheimer's disease, amyotrophic lateral sclerosis, Huntington's disease, and Parkinson's disease).

In another aspect, the RXR indication is a liver indication.

Examples of liver indications include liver inflammation.

In another aspect, the RXR indication is a non-CNS, non-liver indication.

Examples of peripheral, non-liver indications include inflammation, inflammatory bowel disease, and rheumatoid arthritis.

### LXR Side Effects/Adverse Events

In another aspect, the side effect is a non-CNS side effect. These side effects can be separated into non-liver side effects and liver side effects. It is noted that non-liver side effects and liver side effects can result from the same LXR agonist.

In another aspect, the side effect is a non-liver side effect.

Examples of non-liver side effects include decreased neutrophil count and dyslipidemias including increased LDL-C (low-density lipoprotein cholesterol), increased plasma triglycerides and decreased HDL-C (high-density lipoprotein cholesterol). Additional examples include gastrointestinal side effects.

In another aspect, the side effect is a liver side effect.

Examples of liver side effects include increased hepatic triglycerides and hepatic steatosis.

### Additional Methods

In another aspect, the Inhibitor compound is selected from:
i) an LXR Antagonist,
ii) an LXR Inverse Agonist, and,
iii) a combination thereof.

In another aspect, the Inhibitor compound is an LXR Antagonist.

In another aspect, the LXR Antagonist meets the following criteria:
(a) LXRb RLB Ki <100 nM;
(b) LXR Antagonist Reporter assay, EC50 <300 nM; and,
(c) Free drug exposure Cmax in mouse liver >in vitro EC50.

In another aspect, the Inhibitor compound is an LXR Inverse Agonist.

In another aspect, the LXR Inverse Agonist meets the following criteria:
(a) LXRb RLB Ki <100 nM;
(b) LXR Antagonist Reporter assay EC50 <300 nM; and,
(a) Free drug exposure Cmax in mouse liver >in vitro EC50.

In another aspect, the LXR Agonist is peripherally restricted.

In another aspect, the RXR Agonist is peripherally restricted.

In another aspect, the Inhibitor compound is peripherally restricted.

In another aspect, both the LXR Agonist and the Inhibitor compound are peripherally restricted.

In another aspect, both the RXR Agonist and the Inhibitor compound are peripherally restricted.

In another aspect, the Inhibitor compound is a peripherally restricted LXR Antagonist.

In another aspect, the Inhibitor compound is a peripherally restricted LXR Inverse Agonist.

In another aspect, the Inhibitor compound is a combination of both of an LXR Antagonist and an LXR Inverse Agonist.

In another aspect, the Inhibitor compound has liver tissue specificity.

In another aspect, the Inhibitor compound is an LXR Antagonist that has liver tissue specificity.

In another aspect, the Inhibitor compound is an LXR Inverse Agonist that has liver tissue specificity.

In another aspect, the LXR Inverse Agonist is selected from:

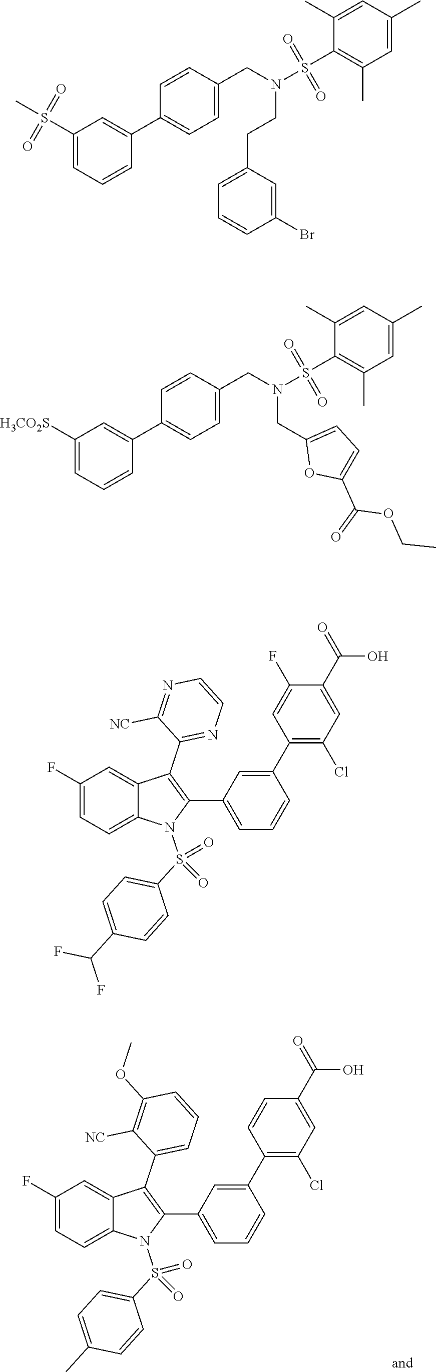

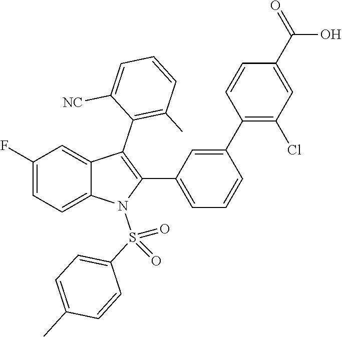 [SMILES: Cc1ccc(S(=O)(=O)n2c(-c3cccc(-c4ccc(C(=O)O)cc4Cl)c3)c(-c3c(C)cccc3C#N)c3cc(F)ccc32)cc1]

or a pharmaceutically acceptable salt thereof.

When the LXR agonist is selected to treat a CNS indication (e.g., GBM), the side effects are typically non-CNS (e.g., systemic non-liver side effects and/or liver side effects). The Inhibitor compound can then be selected to specifically address the liver side effects (e.g., an Inhibitor that has liver tissue specificity), the non-liver side effects (e.g., an Inhibitor that has non-liver tissue specificity), or both types of side effects (e.g., an Inhibitor that is present in the liver and non-liver tissues).

When the LXR agonist is selected to treat a non-CNS, non-liver indication (e.g., atherosclerosis) the side effects are often seen in the liver. So, the Inhibitor compound can then be selected to address these liver side effects.

When the LXR agonist is selected to treat a liver indication (e.g., hepatic steatosis), the side effects are often seen in the non-liver organs. So, the Inhibitor compound can be selected to address these non-liver side effects.

When the RXR agonist is selected to treat a CNS indication (e.g., Alzheimer's disease), the side effects are typically non-CNS (e.g., systemic non-liver side effects and/or liver side effects). The Inhibitor compound can then be selected to specifically address the liver side effects (e.g., an Inhibitor that has liver tissue specificity), the non-liver side effects (e.g., an Inhibitor that has non-liver tissue specificity), or both types of side effects (e.g., an Inhibitor that is present in the liver and non-liver tissues).

When the RXR agonist is selected to treat a non-CNS, non-liver indication (e.g., atherosclerosis) the side effects are often seen in the liver. So, the Inhibitor compound can then be selected to address these liver side effects.

When the RXR agonist is selected to treat a liver indication (e.g., liver inflammation), the side effects are often seen in the non-liver organs. So, the Inhibitor compound can be selected to address these non-liver side effects.

In another aspect, the invention involves compounds and/or compositions for use in medical therapy.

In another aspect, the invention involves the use of compounds for the manufacture of a medicament for the treatment of an indication described herein.

In another aspect, the invention involves a medicament, comprising:
(a) one or more Agonists selected from LXR Agonists and RXR Agonists; and,
(b) one or more Inhibitor compounds.

In another aspect, the invention involves a medicament, comprising:
(a) one or more LXR Agonists; and,
(b) one or more Inhibitor compounds.

In another aspect, the invention involves a medicament, comprising:
(a) one or more RXR Agonists; and,
(b) one or more Inhibitor compounds.

In another aspect, the invention involves pharmaceutical compositions, comprising:
(a) an Agonist selected from an LXR Agonist and an RXR Agonist;
(b) an LXR agonist Inhibitor compound; and,
(c) optionally a pharmaceutically acceptable carrier.

In another aspect, the invention involves pharmaceutical compositions, comprising an LXR agonist and an LXR agonist Inhibitor compound and optionally a pharmaceutically acceptable carrier.

In another aspect, the invention involves pharmaceutical compositions, comprising an RXR agonist and an LXR agonist Inhibitor compound and optionally a pharmaceutically acceptable carrier.

In another aspect, the invention involves pharmaceutical compositions, comprising:
(d) one or more Agonists selected from LXR Agonists and RXR Agonists;
(e) one or more Inhibitor compounds; and,
(f) a pharmaceutically acceptable carrier.

In another aspect, the invention involves pharmaceutical compositions, comprises: one or more LXR Agonists;

In another aspect, the invention involves pharmaceutical compositions, comprises: one or more RXR Agonists;

In another aspect, the invention involves a method of using one or more LXR agonists in combination with one or more Inhibitor compounds.

In another aspect, the invention involves a method of using one or more RXR agonists in combination with one or more Inhibitor compounds.

In another aspect, the invention involves a method of using a single LXR agonist in combination with a single Inhibitor compound.

In another aspect, the invention involves a method of using a single RXR agonist in combination with a single Inhibitor compound.

In another aspect, the invention involves a method of using a single LXR agonist in combination with one or more Inhibitor compounds.

In another aspect, the invention involves a method of using a single RXR agonist in combination with one or more Inhibitor compounds.

In another aspect, the invention involves a method of using one or more LXR agonists in combination with a single Inhibitor compound.

In another aspect, the invention involves a method of using one or more RXR agonists in combination with a single Inhibitor compound.

In another aspect, the method of treatment is applied to a mammal. In another aspect, the mammal is a human being.

In another aspect, the use of the Inhibitor alleviates some or all the side effects associated with use of the LXR Agonist.

In another aspect, the use of the Inhibitor alleviates some or all the side effects associated with use of the RXR Agonist.

In another aspect, use of the Inhibitor allows for a higher maximum tolerated dose of the LXR Agonist.

In another aspect, use of the Inhibitor allows for a higher maximum tolerated dose of the RXR Agonist.

In another aspect, the Agonist and Inhibitor are in the same dosage form or dosage vehicle.

In another aspect, the Agonist and Inhibitor are in separate dosage forms or dosage vehicles.

In another aspect, the Agonist and Inhibitor are in an immediate release dosage form.

In another aspect, Agonist and Inhibitor are in a controlled release dosage form.

In another aspect, either the Agonist or the Inhibitor is in an immediate release dosage form, while the other is in a controlled release dosage form.

In another aspect, the dosage forms further comprise one or more pharmaceutically acceptable carriers.

In another aspect, the dosage forms further comprise one or more pharmaceutically acceptable salts.

In another aspect, the Agonist and Inhibitor can be taken orally. In this aspect, the Agonist and Inhibitor can be given orally in tablets, troches, liquids, drops, capsules, caplets and gel caps or other such formulations known to one skilled in the art. In other aspect, another route of administration is used. These routes include parenteral, topical, transdermal, ocular, rectal, sublingual, and vaginal.

In another aspect, the invention involves a medicament comprising both an Agonist and an Inhibitor. In another aspect, the medicament can be taken orally. The medicament may be given orally in tablets, troches, liquids, drops, capsules, caplets and gel caps or other such formulations known to one skilled in the art. In other aspect, another route of administration is used. These routes include parenteral, topical, transdermal, ocular, rectal, sublingual, and vaginal.

In another aspect, the Agonist is administered sequentially with the Inhibitor.

In another aspect, the Agonist is administered concurrently with the Inhibitor.

In another aspect, the Agonist is administered prior to administration of the Inhibitor.

In another aspect, the Inhibitor is administered prior to administration of the Agonist.

In another aspect, the Inhibitor is administered within one hour of administration of the Agonist. Further examples include administering within 30 minutes, 10 minutes, 1 minute, and 30 seconds.

In another aspect, prior to the start of a drug regimen of the type outlined above, there may be a lead-in period that lasts from one to fourteen days. During this lead-in period, the Inhibitor may be given by itself prior to the start of administration of the combination.

In another aspect, from 10 micrograms to 10 grams of Agonist is used in the combination with the Inhibitor. Further examples include from 1 mg to 1 g, 5-500 mg, and 2.5-200 mg Agonist is used in combination with the Inhibitor.

In another aspect from 10 micrograms to 10 grams of Inhibitor is used in the combination with the Agonist. Further examples include from 1 mg to 1 g, 5-500 mg, and 2.5-200 mg is used in combination with the Agonist.

In another aspect, the Agonist and Inhibitor are administered to a patient 6 times during a 24-hour period. Further examples include 5, 4, 3, 2, and 1 time during a 24 hour period.

In another aspect, the Inhibitor is administered for one or more dose periods prior to administration of the Agonist to allow for accumulation of the Inhibitor in the body, or for the Inhibitor to reach or approach steady state exposure levels. This accumulation, or higher exposure levels of the Inhibitor, could increase the blockade of LXR/RXR receptors and reduce adverse events (e.g., side effects) upon initiation of dosing of the Agonist. In another aspect, the Inhibitor is administered for one or more days prior to administration of the LXR.

### Examples of RXR Agonists

In another aspect, the compound is:

Bexarotene (LGD1069)

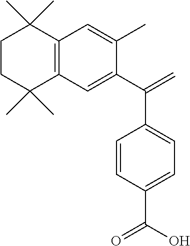 [SMILES: C=C(c1ccc(C(=O)O)cc1)c1cc2c(cc1C)C(C)(C)CCC2(C)C]

or a pharmaceutically acceptable salt thereof.

### Examples of LXR Agonists

In another aspect, the compound is:

RGX-104

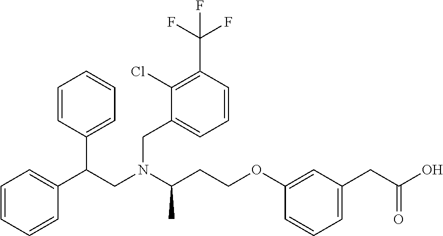 [SMILES: C[C@H](CCOc1cccc(CC(=O)O)c1)N(Cc1cccc(C(F)(F)F)c1Cl)CC(c1ccccc1)c1ccccc1]

or a pharmaceutically acceptable salt thereof.

In another aspect, the compound is:

GW3965

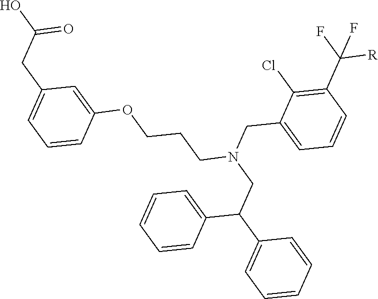

or a pharmaceutically acceptable salt thereof.

In another aspect, the compound is:

T0901317

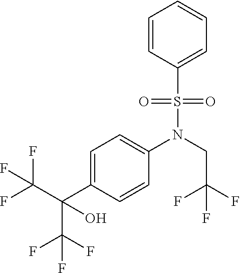 [SMILES: O=S(=O)(c1ccccc1)N(CC(F)(F)F)c1ccc(C(O)(C(F)(F)F)C(F)(F)F)cc1]

or a pharmaceutically acceptable salt thereof.

In another aspect, the compound is:

LXR-623 (brain penetrant)

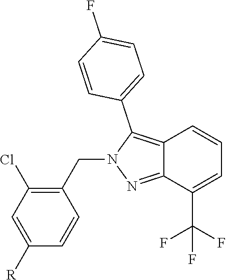

or a pharmaceutically acceptable salt thereof.

In another aspect, the compound is a described in U.S. Pat. No. 9,751,869 (see Formula I and II below).

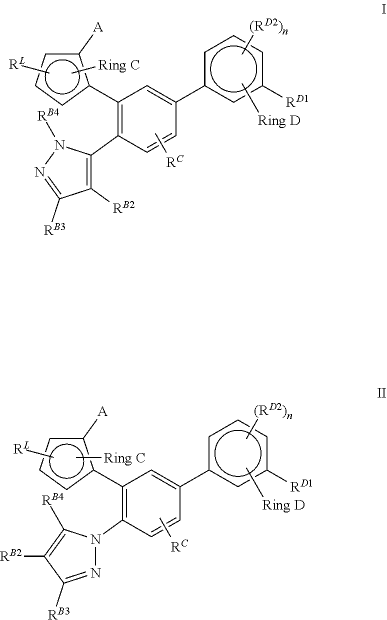

In another aspect, examples include a compound selected from the group:
1,1-difluoro-1-(1-methyl-5-(3′-(methylsulfonyl)-3-(5-(4-trifluoromethoxy)phenyl)-1H-1,2,3-triazol-1-yl)biphenyl-4-yl)-1H-pyrazol-3-yl)propan-2-ol
(4′-(3-(1,1-difluoropropyl)-1-methyl-1H-pyrazol-5-yl)-3-fluoro-5-(methylsulfonyl)-3′-(5-(4-(trifluoromethoxy)phenyl)-1H-1,2,3-triazol-1-yl)biphenyl-4-yl)-1H-pyrazol
1,1-difluoro-1-(5-(3′-fluoro-4′-(hydroxymethyl)-5′-(methylsulfonyl)-3-(5-(4-(trifluoromethoxy)phenyl)-1H-1,2,3-triazol-1-yl)biphenyl-4-yl)-1-methyl-1H-pyrazol-3-yl)-2-methylpropan-2-ol
2,2-difluoro-2-(1-methyl-5-(3′-(methylsulfonyl)-3-(5-(4-trifluoromethoxy)phenyl)-1H-1,2,3-triazol-1-yl)biphenyl-4-yl)-1H-pyrazol-3-yl)acetamide
((4′-(3-(1,1-difluoroethyl)-1,4-dimethyl-1H-pyrazol-5-yl)-3-fluoro-5-(methylsulfonyl)-3′-(5-(4-trifluoromethoxy)phenyl)-1H-1,2,3-triazol-1-yl)biphenyl-4-yl)methanol
1,1-difluoro-1-(5-(3′-fluoro-4′-(hydroxymethyl)-5′-(methylsulfonyl)-3-(5-(4-(trifluoromethoxy)phenyl)-1H-1,2,3-triazol-1-yl)biphenyl-4-yl)-1-methyl-1H-pyrazol-3-yl)propan-2-ol
1,1-difluoro-2-methyl-1-(1-methyl-5-3′-(methylsulfonyl)-3-(5-(4-(trifluoromethoxy)phenyl)-1H-1,2,3-triazol-1-yl)biphenyl-4-yl)-1H-pyrazol-3-yl)propan-2-ol
2,2-difluoro-2-(5-(3′-fluoro-4′-(hydroxymethyl)-5′-(methylsulfonyl)-3-(5-(4-(trifluoromethoxy)phenyl)-1H-1,2,3-triazol-1-yl)biphenyl-4-yl)-1-methyl-1H-pyrazol-3-yl)acetamide
1-(4-(3-(1,1-difluoropropyl)-1-methyl-1H-pyrazol-5-yl)-3′-methylsulfonyl)biphenyl-3-yl)-5-(4-(trifluoromethoxy)phenyl)-1H-1,2,3-triazole
1-(4-(3-(1,1-difluoroethyl)-1,4-dimethyl-1H-pyrazol-5-yl)-3′-(methylsulfonyl)biphenyl-3-yl)-5-(4-trifluoromethoxy)phenyl)-1H-1,2,3-triazole
(4′-(3-(1,1-difluoroethyl)-1,4-dimethyl-1H-pyrazol-5-yl)-3-(methylsulfonyl)-3′-(5-(4-(trifluoromethoxy)phenyl)-1H-1,2,3-triazol-1-yl)biphenyl-4-yl)methanol
2-methyl-2-{4′-[1-methyl-3-(trifluoromethyl)-1H-pyrazol-5-yl]-3′-(5-{4-[(trifluoromethyl)oxy]phenyl} 1H-1,2,3-triazol-1-yl)biphenyl-3-yl}propanamide
[3-chloro-5-(methylsulfonyl)-4′-[1-methyl-3-(trifluoromethyl)-1H-pyrazol-5-yl]-3′-(5-{4-[(trifluoromethyl)oxy]phenyl}-1H-1,2,3-triazol-1-yl)biphenyl-4-yl]methanol
1-{3′-(methylsulfonyl)-4-[1-methyl-3-(trifluoromethyl)-1H-pyrazol-5-yl]biphenyl-3-yl}-5-{4-[(trifluoromethyl)oxy]phenyl}-1H-1,2,3-triazole
(3-fluoro-4′-(1-methyl-3-(trifluoromethyl)-1H-pyrazol-5-yl)-5-methylsulfonyl)-3′-(5-(4-(trifluoromethoxy)phenyl)-1H-1,2,3-triazol-1-yl)biphenyl-4-yl)methanol
1-[4-{3-[1,1-difluoro-2-(methyloxy)ethyl]-1-methyl-1H-pyrazol-5-yl}3′-(methylsulfonyl)biphenyl-3-yl]-5-{4-[(trifluoromethyl)oxy]phenyl}-1H-1,2,3-triazole
[4′-{3-[1,1-difluoro-2-methyloxy)ethyl]-1-methyl-1H-pyrazol-5-yl}-3-fluoro-5-(methylsulfonyl)-3′-(5-{4-[(trifluoromethyl)oxy]phenyl}-1H-1,2,3-triazol-1-yl)biphenyl-4-yl]methanol
1-{4-[3-(1,1-difluoroethyl)-1-methyl-1H-pyrazol-5-yl]-3′-(methylsulfonyl)biphenyl-3-yl}-5-{4-[(trifluoromethyl)oxy]phenyl}-1H-1,2,3-triazole 4′-[3-(1,1-difluoroethyl)-1-methyl-1H-pyrazol-5-yl]-N,N-dimethyl-3′-(5-{4-[(trifluoromethyl)oxy]phenyl}-1H-1,2,3-triazol-1-yl)biphenyl-3-sulfonamide
4′-[3-(1,1-difluoroethyl)-1-methyl-1H-pyrazol-5-yl]-N-methyl-3′-(5-{4-[(trifluoromethyl)oxy]phenyl}-1H-1,2,3-triazol-1-yl)biphenyl-3-sulfonamide
{4′-[3-(1,1-difluoroethyl)-1-methyl-1H-pyrazol-5-yl]-3-fluoro-5-methylsulfonyl)-3′-(5-{4-trifluoromethyl)oxy]phenyl}-1H-1,2,3-triazol-1-yl)biphenyl-4-yl}methanol
1-{4-[3-(difluoromethyl)-1-methyl-1H-pyrazol-5-yl-3′-(methylsulfonyl)biphenyl-3-yl}-5-{4-[(trifluoromethyl)oxy]phenyl}-1H-1,2,3-triazole
{4′-[3-(difluoromethyl)-1-methyl-1H-pyrazol-5-yl]-3-fluoro-5-methylsulfonyl)-3′-(5-{4-[(trifluoromethyl)oxy]phenyl}-1H-1,2,3-triazol-1-yl)biphenyl-4-yl}methanol
2-(5-(3-(5-(4-chlorophenyl)-1H-1,2,3-triazol-1-yl)-3′-(methylsulfonyl)-[1,1′-biphenyl]-4-yl)-1-methyl-1H-pyrazol-3-yl)-2,2-difluoroethanol
2,2-difluoro-2-(1-methyl-5-(3′-(methylsulfonyl)-3-(5-(4-(trifluoromethoxy)phenyl)-1H-1,2,3-triazol-1-yl)-1,1′-biphenyl]-4-yl)-1H-pyrazol-3-yl)ethanol
2,2-difluoro-2-(5-(3′-fluoro-4′-(hydroxymethyl)-5′-(methylsulfonyl)-3-(5-(4-(trifluoromethoxy)phenyl)-1H-1,2,3-triazol-1-yl)-[1,1′-biphenyl]-4-yl)-1-methyl-1H-pyrazol-3-yl)ethanol
2,2-difluoro-2-(5-(3′-fluoro-4′-(hydroxymethyl)-5′-(methylsulfonyl)-3-(5-(4-(trifluoromethoxy)phenyl)-1H-1,2,3-triazol-1-yl)-[1,1′-biphenyl]-4-yl)-1-methyl-1H-pyrazol-3-yl)-N-(1-hydroxy-2-methylpropan-2-yl)acetamide
2,2-difluoro-N-(1-hydroxy-2-(methylsulfonyl)-3-(5-(4-trifluoromethoxy)phenyl)-1H-1,2,3-triazol-1-yl)-[1,1′-biphenyl]-4-yl)-1H-pyrazol-3-yl)acetamide
2-((2,2-difluoro-2-(1-methyl-5-(3′-(methylsulfonyl)-3-(5-(4-trifluoromethoxy)phenyl)-1H-1,2,3-triazol-1-yl)-[1,1′-biphenyl]-4-yl)-1H-pyrazol-3-yl)ethyl)amino)ethanol
2,2-difluoro-2-(1-methyl-5-(3′-(methylsulfonyl)-3-(5-(4-trifluoromethoxy)phenyl)-1H-1,2,3-triazol-1-yl)-[1,1′-biphenyl]-4-yl)-1H-pyrazol-3-yl)acetonitrile
1-(5-(3-(5-(4-chlorophenyl)-1H-1,2,3-triazol-1-yl)-3′-(methylsulfonyl)-[1,1′-biphenyl]-4-yl)-1-methyl-1H-pyrazol-3-yl)-1,1-difluoro-2-methylpropan-2-ol
2-(5-(3-(5-(2,4-dichlorophenyl)-1H-1,2,3-triazol-1-yl)-3′-(methylsulfonyl)-[1,1′-biphenyl]-4-yl)-1-methyl-1H-pyrazol-3-yl)-2,2-difluoroacetamide
1-(5-(3-(5-(2,2-difluorobenzo[d][1,3]dioxol-5-yl)-1H-1,2,3-triazol-1-yl)-3′-(methylsulfonyl)-[1,1′-biphenyl]-4-yl)-1-methyl-1H-pyrazol-3-yl)-1,1-difluoro-2-methylpropan-2-ol
1-(5-(3-(5-(2,2-difluorobenzo[d][1,3]dioxol-5-yl)-1H-1,2,3-triazol-1-yl)-3′-fluoro-4′-(hydroxymethyl)-5′-(methylsulfonyl)-[1,1′-biphenyl]-4-yl)-1-methyl-1H-pyrazol-3-yl)-1,1-difluoro-2-methylpropan-2-ol
2-(5-(3-(5-(2,2-difluorobenzo[d][1,3]dioxol-5-yl)-1H-1,2,3-triazol-1-yl)-3′-(methylsulfonyl)-[1,1′-biphenyl]-4-yl)-1-methyl-1H-pyrazol-3-yl)-2,2-difluoroethanol
2-(5-(3-(5-(2,2-difluorobenzo[d][1,3]dioxol-5-yl)-1H-(hydroxymethyl)-5′-(methylsulfonyl)-[1,1′-biphenyl]-4-yl)-1-methyl-1H-pyrazol-3-yl)-2,2-difluoroethanol
N-(1-cyano-1-methylethyl)-1-methyl-5-[3′-(methylsulfonyl)-3-(5-{4-[(trifluoromethyl)oxy]phenyl1-1H-1,2,3-triazol-1-yl)biphenyl-4-yl]-1H-pyrazole-3-carboxamide
N,N,1-trimethyl-5-[3′-(methylsulfonyl)-3-(5-{4-[(trifluoromethyl)oxy]phenyl]-1H-1,2,3-triazol-1-yl)biphenyl-4-yl]-1H-pyrazole-3-carboxamide
1-methyl-5-[3′-(methylsulfonyl)-3-(5-{4-[(trifluoromethyl)oxy]phenyl}-1H-1,2,3-triazol-1-yl)biphenyl-4-yl]-N-(2,2,2-trifluoroethyl)-1H-pyrazole-3-carboxamide
N-(1,1-dimethylethyl)-1-methyl-5-[3′-(methylsulfonyl)-3-(5-{4-[(trifluoromethyl)oxy]phenyl}-1H-1,2,3-triazol-1-yl)biphenyl-4-yl]-1H-pyrazole-3-carboxamide
2-(5-(3-(4-(2,2-difluorobenzo[d][1,3]dioxol-5-yl)-2-methyloxazol-5-yl)-3′-fluoro-5′-methylsulfonyl)biphenyl-4-yl)-1-methyl-1H-pyrazol-3-yl)-2,2-difluoroacetamide
2-(5-(3-(2-cyclopropyl-4-(2,2-difluorobenzo[d][1,3]dioxol-5-yl)oxazol-5-yl)-3′-fluoro-4′-(hydroxymethyl)-5′-(methylsulfonyl)biphenyl-4-yl)-1-methyl-1H-pyrazol-3-yl)-2,2-difluoroacetamide
2-(5-(3-(4-(2,2-difluorobenzo[d][1,3]dioxol-5-yl)-2-methyloxazol-5-yl)-3′-fluoro-4′-(hydroxymethyl)-5′-methylsulfonyl)biphenyl-4-yl)-1-methyl-1H-pyrazol-3-yl)-2,2-difluoroacetonitrile
1,1-difluoro-2-methyl-1-(1-methyl-5-(3-(2-methyl-4-(4-(trifluoromethoxy)phenyl)oxazol-5-yl)-3′-(methylsulfonyl)biphenyl-4-yl)-1H-pyrazol-3-yl)propan-2-ol
2-(5-(3-(4-(2,2-difluorobenzo[d][1,3]dioxol-5-yl)-2-methyloxazol-5-yl)-3′-(methylsulfonyl)biphenyl-4-yl)-1-methyl-1H-pyrazol-3-yl)-2,2-difluoroethanol
2-(5-(3-(4-(2,2-difluorobenzo[d][1,3]dioxol-5-yl)-2-methyloxazol-5-yl)-3′-fluoro-4′-(hydroxymethyl)-5′-methylsulfonyl)biphenyl-4-yl)-1-methyl-1H-pyrazol-3-yl)-2,2-difluoro-N-methylacetamide
(4′(3-(difluoro(1,3,4-oxadiazol-2-yl)methyl)-1-methyl-1H-pyrazol-5-yl)-3′-(4-(2,2-difluorobenzo[d][1,3]dioxol-5-yl)-2-methyloxazol-5-yl)-3-fluoro-5-(methylsulfonyl)biphenyl-4-yl)methanol
(4′-(3-(difluoro(4H-1,2,4-triazol-3-yl)methyl)-1-methyl-1H-pyrazol-5-yl)-3′-(4-(2,2-difluorobenzo[d][1,3]dioxol-5-yl)-2-methyloxazol-5-yl)-3-fluoro-5-(methylsulfonyl)biphenyl-4-yl)methanol
2-methyl-5-(4-(1-methyl-3-(trifluoromethyl)-1H-pyrazol-5-yl)-3′-(methylsulfonyl)biphenyl-3-yl)-4-(4-(trifluoromethoxy)phenyl)Oxazole
2-(5-(3-(4-(2,2-difluorobenzo[d][1,3]dioxol-5-yl)-2-methyloxazol-5-yl)-3′-(methylsulfonyl)biphenyl-4-yl)-1-methyl-1H-pyrazol-3-yl)propan-2-ol
N-(2-cyanopropan-2-yl)-5-(3-(4-(2,2-difluorobenzo[d][1,3]dioxol-5-yl)-2-methyloxazol-5-yl)-3′-fluoro-4′-(hydroxymethyl)-5′-(methylsulfonyl)biphenyl-4-yl)-1-methyl-1H-pyrazole-3-carboxamide
5-(3-(4-(2,2-difluorobenzo[d][1,3]dioxol-5-yl)-2-methyloxazol-5-yl)-3′-fluoro-4′-(hydroxymethyl)-5′-(methylsulfonyl)biphenyl-4-yl)-1-methyl-1H-pyrazole-3-carboxamide
(3′-(4-(42,2-difluorobenzo[d][1,3]dioxol-5-yl)-2-methyloxazol-5-yl)-3-fluoro-4′-(1-methyl-3-(oxazol-5-yl)-1H-pyrazol-5-yl)-5-(methylsulfonyl)-[1,1′-biphenyl]-4-yl)methanol
2-(5-(3-(4-(2,2-difluorobenzo[d][1,3]dioxol-5-yl)-2-methyloxazol-5-yl)-3′-fluoro-4′-(hydroxymethyl)-5′-(methylsulfonyl)-[1,1′-biphenyl]-4-yl)-1-methyl-1H-pyrazol-3-yl)acetonitrile
(S)-2-(5-(3-(4-(2,2-difluorobenzo[d][1,3]dioxol-5-yl)-2-methyloxazol-5-yl)-3′-fluoro-4′-(hydroxymethyl)-5′-(methylsulfonyl)-[1,1′-biphenyl]-4-yl)-1-methyl-1H-pyrazol-3-yl)propanenitrile
1-(5-(3-(4-(2,2-difluorobenzo[d][1,3]dioxol-5-yl)-2-methyloxazol-5-yl)-3′-fluoro-4′-(hydroxymethyl)-5′-(methylsulfonyl)-[1,1′-biphenyl]-4-yl)-1-methyl-1H-pyrazol-3-yl)cyclopropanecarbonitrile
2-(5-(3-(4-(2,2-difluorobenzo[d][1,3]dioxol-5-yl)-2-methyloxazol-5-yl)-3′-fluoro-4′-(hydroxymethyl)-5′-(methylsulfonyl)-[1,1′-biphenyl]-4-yl)-1-methyl-1H-pyrazol-3-yl)acetamide
2-(5-(3-(5-(2,2-difluorobenzo[d][1,3]dioxol-5-yl)-2-methyloxazol-4-yl)-3′-fluoro-4′-(hydroxymethyl)-5′-(methylsulfonyl)biphenyl-4-yl)-1-methyl-1H-pyrazol-3-yl)-2-methylpropan-1-ol
2-(5-(3-(5-(2,2-difluorobenzo[d][1,3]dioxol-5-yl)-2-methyloxazol-4-yl)-3′-fluoro-4′-(hydroxymethyl)-5′-(methylsulfonyl)biphenyl-4-yl)-1-methyl-1H-pyrazol-3-yl)-2-methylpropanamide
3-(5-(3-(5-(2,2-difluorobenzo[d][1,3]dioxol-5-yl)-2-methyloxazol-4-yl)-3′-fluoro-4′-(hydroxymethyl)-5′-methylsulfonyl)biphenyl-4-yl)-1-methyl-1H-pyrazol-3-yl)-2,3-dimethylbutan-2-ol
2-(5-(3-(4-(2,2-difluorobenzo[d][1,3]dioxol-5-yl)-2-methyloxazol-5-yl)-3′-fluoro-4′-(hydroxymethyl)-5′-methylsulfonyl)biphenyl-4-yl)-1-methyl-1H-pyrazol-3-yl)-2-methylpropanamide
3-(5-(3-(4-(2,2-difluorobenzo[d][1,3]dioxol-5-yl)-2-methyloxazol-5-yl)-3′-fluoro-4′-(hydroxymethyl)-5′-(methylsulfonyl)biphenyl-4-yl)-1-methyl-1H-pyrazol-3-yl)-2,3-dimethylbutan-2-ol
2-(5-(3-(4-(2,2-difluorobenzo[d][1,3]dioxol-5-yl)-2-methyloxazol-5-yl)-3′-fluoro-4′-(hydroxymethyl)-5′-(methylsulfonyl)biphenyl-4-yl)-1-methyl-1H-pyrazol-3-yl)-2-methylpropanenitrile
(4′-(3-(2-(1,3,4-oxadiazol-2-yl)propan-2-yl)-1-methyl-1H-pyrazol-5-yl)-3′-(4-(2,2-difluorobenzo[d][1,3]dioxol-5-yl)-2-methyloxazol-5-yl)-3-fluoro-5-(methylsulfonyl)biphenyl-4-yl)methanol
1-(5-(3-(4-(2,2-difluorobenzo[d][1,3]dioxol-5-yl)-2-methyloxazol-5-yl)-3′-fluoro-4′-(hydroxymethyl)-5′-(methylsulfonyl)-[1,1′-biphenyl]-4-yl)-1-methyl-1H-pyrazol-3-yl)-N,N-dimethylmethanesulfonamide
2,2-difluoro-2-(5-(3′-fluoro-4′-(hydroxymethyl)-3-(2-methyl-4-(4-(trifluoromethoxy)phenyl)oxazol-5-yl)-5′-(methylsulfonyl)biphenyl-4-yl)-1-methyl-1H-pyrazol-3-yl)acetamide
2-(5-(3-(4-(4-(difluoromethoxy)-3-fluorophenyl)-2-methyloxazol-5-yl)-3′-fluoro-4′-(hydroxymethyl)-5′-(methylsulfonyl)biphenyl-4-yl)-1-methyl-1H-pyrazol-3-yl)-2,2-difluoroacetamide
2-(5-(3-(4-(4-(difluoromethoxy)-3-fluorophenyl)-2-methyloxazol-5-yl)-3′-methylsulfonyl)biphenyl-4-yl)-1-methyl-1H-pyrazol-3-yl)-2,2-difluoroacetamide
2-(5-(3-(4-(2,2-difluorobenzo[d][1,3]dioxol-5-yl)-2-methyloxazol-5-yl)-3′-(methylsulfonyl)biphenyl-4-yl)-1-methyl-1H-pyrazol-3-yl)-2,2-difluoroacetamide
2-(5-(3-(4-(2,2-difluorobenzo[d][1,3]dioxol-5-yl)-2-methyloxazol-5-yl)-3′-fluoro-4′-(hydroxymethyl)-5′-methylsulfonyl)biphenyl-4-yl)-1-methyl-1H-pyrazol-3-yl)-2,2-difluoroacetamide
2-(5-(3-(4-(2,2-difluorobenzo[d][1,3]dioxol-5-yl)-2-methyloxazol-5-yl)-3′-fluoro-4′-(hydroxymethyl)-5′-(methylsulfonyl)biphenyl-4-yl)-1-methyl-1H-pyrazol-3-yl)-2,2-difluoroacetic acid
2-(5-(3-(4-(4-(difluoromethoxy)-3-fluorophenyl)-2-methyloxazol-5-yl)-3′-(methylsulfonyl)biphenyl-4-yl)-1-methyl-1H-pyrazol-3-yl)-2,2-difluoroethanol
2-(5-(3-(4-(4-(difluoromethoxy)-3-fluorophenyl)-2-methyloxazol-5-yl)-3′-fluoro-4′-(hydroxymethyl)-5′-(methylsulfonyl)biphenyl-4-yl)-1-methyl-1H-pyrazol-3-yl)-2,2-difluoroethanol
2-(5-(3-(4-(4-(difluoromethoxy)-3-fluorophenyl)-2-methyloxazol-5-yl)-3′-fluoro-4′-(hydroxymethyl)-5′-(methylsulfonyl)biphenyl-4-yl)-1-methyl-1H-pyrazol-3-yl)-2,2-difluoro-N-methylacetamide
2-(5-(3-(4-(4-(difluoromethoxy)-3-fluorophenyl)-2-methyloxazol-5-yl)-3′-fluoro-4′-(hydroxymethyl)-5′-(methylsulfonyl)biphenyl-4-yl)-1-methyl-1H-pyrazol-3-yl)-2,2-difluoro-N,N-dimethylacetamide
2-(5-(3-(4-(2,2-difluorobenzo[d][1,3]dioxol-5-yl)-2-methyloxazol-5-yl)-3′-fluoro-4′-(hydroxymethyl)-5′-(methylsulfonyl)biphenyl-4-yl)-1-methyl-1H-pyrazol-3-yl)-2,2-difluoro-N,N-dimethylacetamide
2-(5-(3-(4-(2,2-difluorobenzo[d][1,3]dioxol-5-yl)-2-methyloxazol-5-yl)-3′-fluoro-4′-OH(hydroxymethyl)-5′-methylsulfonyl)biphenyl-4-yl)-1-methyl-1H-pyrazol-3-yl)-2,2-difluoro-1-morpholinoethanone
2-(5-(3-(4-(2,2-difluorobenzo[d][1,3]dioxol-5-yl)-2-methyloxazol-5-yl)-3′-fluoro-4′-(hydroxymethyl)-5′-(methylsulfonyl)biphenyl-4-yl)-1-methyl-1H-pyrazol-3-yl)-2,2-difluoro-N-isopropylacetamide
2-(5-(3-(4-(2,2-difluorobenzo[d][1,3]dioxol-5-yl)-2-methyloxazol-5-yl)-3′-fluoro-4′-(hydroxymethyl)-5′-(methylsulfonyl)-[1,1′-biphenyl]-4-yl)-1-methyl-1H-pyrazol-3-yl)-2,2-difluoro-1-(pyrrolidin-1-yl)ethanone
N-tert-butyl-2-(5-(3-(4-(2,2-difluorobenzo[d][1,3]dioxol-5-yl)-2-methyloxazol-5-yl)-3′-fluoro-4′-(hydroxymethyl)-5′-(methylsulfonyl)biphenyl-4-yl)-1-methyl-1H-pyrazol-3-yl)-2,2-difluoroacetamide
2-(5-(3-(4-(2,2-difluorobenzo[d][1,3]dioxol-5-yl)-2-methyloxazol-5-yl)-3′-fluoro-5′-(methylsulfonyl)biphenyl-4-yl)-1-methyl-1H-pyrazol-3-yl)-2,2-difluoro-N-methylacetamide
2-(5-(3-(4-(2,2-difluorobenzo[d][1,3]dioxol-5-yl)-2-methyloxazol-5-yl)-3′-fluoro-4′-(hydroxymethyl)-5′-(methylsulfonyl) biphenyl-4-yl)-1-methyl-1H-pyrazol-3-yl)-2,2-difluoro-N-(2-hydroxyethyl)acetamide
N-(cyclopropylmethyl)-2-(5-(3-(4-(2,2-difluorobenzo[d][1,3]dioxol-5-yl)-2-methyloxazol-5-yl)-3′-fluoro-4′-(hydroxymethyl)-5′-(methylsulfonyl) biphenyl-4-yl)-1-methyl-1H-pyrazol-3-yl)-2,2-difluoroacetamide
N-cyclopropyl-2-(5-(3-(4-(2,2-difluorobenzo[d][1,3]dioxol-5-yl)-2-methyloxazol-5-yl)-3′-fluoro-4′-(hydroxymethyl)-5′-(methylsulfonyl) biphenyl-4-yl)-1-methyl-1H-pyrazol-3-yl)-2,2-difluoroacetamide
N-(2-amino-2-oxoethyl)-2-(5-(3-(4-(2,2-difluorobenzo[d][1,3]dioxol-5-yl)-2-methyloxazol-5-yl)-3′-fluoro-4′-(hydroxymethyl)-5′-(methylsulfonyl) biphenyl-4-yl)-1-methyl-1H-pyrazol-3-yl)-2,2-difluoroacetamide
2-(5-(3-(4-(2,2-difluorobenzo[d][1,3]dioxol-5-yl)-2-methyloxazol-5-yl)-3′-fluoro-4′-(hydroxymethyl)-5′-(methylsulfonyl) biphenyl-4-yl)-1-methyl-1H-pyrazol-3-yl)-2,2-difluoro-N-(2,2,2-trifluoroethyl)acetamide
2-(5-(3-(5-(2,2-difluorobenzo[d][1,3]dioxol-5-yl)-2-methyloxazol-4-yl)-3′-fluoro-4′-(hydroxymethyl)-5′-(methylsulfonyl)biphenyl-4-yl)-1-methyl-1H-pyrazol-3-yl)-2,2-difluoro-N-methylacetamide
2-(5-(3-(4-(2,2-difluorobenzo[d][1,3]dioxol-5-yl)-2-methyloxazol-5-yl)-3′-fluoro-4′-(hydroxymethyl)-5′-(methylsulfonyl) biphenyl-4-yl)-1-methyl-1H-pyrazol-3-yl)propan-2-ol
(3-fluoro-4′-(1-methyl-3-1-(trifluoromethyl)-1H-pyrazol-5-yl)-3′-(2-methyl-4-(4-(trifluoromethoxy) phenyl)oxazol-5-yl)-5-(methylsulfonyl) biphenyl-4-yl)methanol
4-(2,2-difluorobenzo[d][1,3]dioxol-5-yl)-5-(4-(3-(1,1-difluoroethyl)-1-methyl-1H-pyrazol-5-yl)-3′-(methylsulfonyl)biphenyl-3-yl)-2-methyloxazole
(3′-(4-(2,2-difluorobenzo[d][1,3]dioxol-5-yl)-2-methyloxazol-5-yl)-4′-(3-(1,1-difluoroethyl)-1-methyl-1H-pyrazol-5-yl)-3-fluoro-5-(methylsulfonyl) biphenyl-4-yl)methanol
2-(5-(3-(4-(2,2-difluorobenzo[d][1,3]dioxol-5-yl)-2-methyloxazol-5-yl)-3′-(methylsulfonyl) biphenyl-4-yl)-1-methyl-1H-pyrazol-3-yl)-2-methylpropanamide
2-(5-(3-(4-(2,2-difluorobenzo[d][1,3]dioxol-5-yl)-2-methyloxazol-5-yl)-3′-fluoro-4′-(hydroxymethyl)-5′-(methylsulfonyl)biphenyl-4-yl)-1-methyl-1H-pyrazol-3-yl)-2-methylpropanenitrile
(5-(3-(4-(2,2-difluorobenzo[d][1,3]dioxol-5-yl)-2-methyloxazol-5-yl)-3′-(methylsulfonyl)-[1,1′-biphenyl]-4-yl)-1-methyl-1H-pyrazol-3-yl)methanol
(5-(3-(4-(2,2-difluorobenzo[d][1,3]dioxol-5-yl)-2-methyloxazol-5-yl)-3′-fluoro-4′-(hydroxymethyl)-5′-(methylsulfonyl)-[1,1′-biphenyl]-4-yl)-1-methyl-1H-pyrazol-3-yl)methanol
2-(5-(3-(4-(2,2-difluorobenzo[d][1,3]dioxol-5-yl)-2-methyloxazol-5-yl)-3′-(methylsulfonyl)-[1,1′-biphenyl]-4-yl)-1-methyl-1H-pyrazol-3-yl)-1,3,4-oxadiazole
1-(5-(3-(4-(2,2-difluorobenzo[d][1,3]dioxol-5-yl)-2-methyloxazol-5-yl)-3′-(methylsulfonyl)-[1,1′-biphenyl]-4-yl)-1-methyl-1H-pyrazol-3-yl)ethanol
1-(5-(3-(4-(2,2-difluorobenzo[d][1,3]dioxol-5-yl)-2-methyloxazol-5-yl)-3′-(methylsulfonyl)-[1,1′-biphenyl]-4-yl)-1-methyl-1H-pyrazol-3-yl)ethanol
1-(5-(3-(4-(2,2-difluorobenzo[d][1,3]dioxol-5-yl)-2-methyloxazol-5-yl)-3′-fluoro-4′-(hydroxymethyl)-5′-(methylsulfonyl)-[1,1′-biphenyl]-4-yl)-1-methyl-1H-pyrazol-3-yl)ethanol
1-(5-(3-(4-(2,2-difluorobenzo[d][1,3]dioxol-5-yl)-2-methyloxazol-5-yl)-3′-fluoro-4′-(hydroxymethyl)-5′-(methylsulfonyl)-[1,1′-biphenyl]-4-yl)-1-methyl-1H-pyrazol-3-yl)ethanol
2-(5-(3-(4-(2,2-difluorobenzo[d][1,3]dioxol-5-yl)-2-methyloxazol-5-yl)-3′-fluoro-4′-(hydroxymethyl)-5′-(methylsulfonyl)-[1,1′-biphenyl]-4-yl)-1-methyl-1H-pyrazol-3-yl)-2,2-difluoroethanol
2-(5-(3-(4-(2,2-difluorobenzo[d][1,3]dioxol-5-yl)-2-methyloxazol-5-yl)-3′-(methylsulfonyl)-[1,1′-biphenyl]-4-yl)-1-methyl-1H-pyrazol-3-yl)-2,2-difluoroethanol
N-cyclopropyl-5-(3-(4-(2,2-difluorobenzo[d][1,3]dioxol-5-yl)-2-methyloxazol-5-yl)-3′-(methylsulfonyl)-[1,1′-biphenyl]-4-yl)-1-methyl-1H-pyrazole-3-carboxamide
N-cyclopropyl-5-(3-(4-(2,2-difluorobenzo[d][1,3]dioxol-5-yl)-2-methyloxazol-5-yl)-3′-fluoro-4′-(hydroxymethyl)-5′-(methylsulfonyl)-[1,1′-biphenyl]-4-yl)-1-methyl-1H-pyrazole-3-carboxamide
N-(cyclopropylmethyl)-5-(3-(4-(2,2-difluorobenzo[d][1,3]dioxol-5-yl)-2-methyloxazol-5-yl)-3′-(methylsulfonyl)-[1,1′-biphenyl]-4-yl)-1-methyl-1H-pyrazole-3-carboxamide
N-(cyclopropylmethyl)-5-(3-(4-(2,2-difluorobenzo[d][1,3]dioxol-5-yl)-2-methyloxazol-5-yl)-3′-fluoro-4′-(hydroxymethyl)-5′-(methylsulfonyl)-[1,1′-biphenyl]-4-yl)-1-methyl-1H-pyrazole-3-carboxamide
5-(3-(4-(2,2-difluorobenzo[d][1,3]dioxol-5-yl)-2-methyloxazol-5-yl)-3′-(methylsulfonyl)-[1,1′-biphenyl]-4-yl)-1-methyl-N-(2,2,2-trifluoroethyl)-1H-pyrazole-3-carboxamide
5-(3-(4-(2,2-difluorobenzo[d][1,3]dioxol-5-yl)-2-methyloxazol-5-yl)-3′-fluoro-4′-(hydroxymethyl)-5′-(methylsulfonyl)-[1,1′-biphenyl]-4-yl)-1-methyl-N-(2,2,2-trifluoroethyl)-1H-pyrazole-3-carboxamide
N-(tert-butyl)-5-(3-(4-(2,2-difluorobenzo[d][1,3]dioxol-5-yl)-2-methyloxazol-5-yl)-3′-(methylsulfonyl)-[1,1′-biphenyl]-4-yl)-1-methyl-1H-pyrazole-3-carboxamide
N-(tert-butyl)-5-(3-(4-(2,2-difluorobenzo[d][1,3]dioxol-5-yl)-2-methyloxazol-5-yl)-3′-fluoro-4′-(hydroxymethyl)-5′-(methylsulfonyl)-[1,1′-biphenyl]-4-yl)-1-methyl-1H-pyrazole-3-carboxamide
2-(5-(3-(4-(2,2-difluorobenzo[d][1,3]dioxol-5-yl)-2-methyloxazol-5-yl)-3′-fluoro-4′-(hydroxymethyl)-5′-(methylsulfonyl)-[1,1′-biphenyl]-4-yl)-1-methyl-1H-pyrazol-3-yl)propanenitrile
2-(5-(3-(4-(2,2-difluorobenzo[d][1,3]dioxol-5-yl)-2-methyloxazol-5-yl)-3′-methylsulfonyl)[1,1′-biphenyl]-4-yl)-1-methyl-1H-pyrazol-3-yl)propanenitrile
2-(5-(3-(4-(2,2-difluorobenzo[d][1,3]dioxol-5-yl)-2-methyloxazol-5-yl)-3′-(methylsulfonyl)[1,1′-biphenyl]-4-yl)-1-methyl-1H-pyrazol-3-yl)propanenitrile
1-(5-(3-(4-(2,2-difluorobenzo[d][1,3]dioxol-5-yl)-2-methyloxazol-5-yl)-3′-(methylsulfonyl)-[1,1′-biphenyl]-4-yl)-1-methyl-1H-pyrazol-3-yl)cyclopropanecarbonitrile
2-(5-(3-(4-(2,2-difluorobenzo[d][1,3]dioxol-5-yl)-2-methyloxazol-5-yl)-3′-(methylsulfonyl)41,1′-biphenyl]-4-yl)-1-methyl-1H-pyrazol-3-yl)acetamide
1-(5-(3-(4-(2,2-difluorobenzo[d][1,3]dioxol-5-yl)-2-methyloxazol-5-yl)-3′-fluoro-4′-(hydroxymethyl)-5′-(methylsulfonyl)-[1,1′-biphenyl]-4-yl)-1-methyl-1H-pyrazol-3-yl)-2-methylpropan-2-ol
1-(5-(3-(4-(2,2-difluorobenzo[d][1,3]dioxol-5-yl)-2-methyloxazol-5-yl)-3′-(methylsulfonyl)41,1′-biphenyl]-4-yl)-1-methyl-1H-pyrazol-3-yl)-2-methylpropan-2-ol
5-(3-(4-(2,2-difluorobenzo[d][1,3]dioxol-5-yl)-2-methyloxazol-5-yl)-3′-fluoro-4′-(hydroxymethyl)-5′-(methylsulfonyl)-[1,1′-biphenyl]-4-yl)-1-methyl-1H-pyrazole-3-carbonitrile
5-(3-(4-(2,2-difluorobenzo[d][1,3]dioxol-5-yl)-2-methyloxazol-5-yl)-3′-(methylsulfonyl)-[1,1′-biphenyl]-4-yl)-1-methyl-1H-pyrazole-3-carbonitrile
N-(2-hydroxy-2-methylpropyl)-1-methyl-5-(3-(2-methyl-4-(4-(trifluoromethoxy)phenyl)oxazol-5-yl)-3′-(methylsulfonyl)-[1,1′-biphenyl]-4-yl)-1H-pyrazole-3-carboxamide
(3-chloro-4′-(1-methyl-3-(trifluoromethyl)-1H-pyrazol-5-yl)-3′-(2-methyl-4-(4-(trifluoromethoxy)phenyl)oxazol-5-yl)-5-(methylsulfonyl)-[1,1′-biphenyl]-4-yl)methanol
4′-(1-methyl-3-(trifluoromethyl)-1H-pyrazol-5-yl)-3′-(2-methyl-4-(4-(trifluoromethoxy)phenyl)oxazol-5-yl)-[1,1′-biphenyl]-3-sulfonamide
N-methyl-4′-(1-methyl-3-(trifluoromethyl)-1H-pyrazol-5-yl)-3′-(2-methyl-4-(4-(trifluoromethoxy)phenyl)oxazol-5-yl)-[1,1′-biphenyl]-3-sulfonamide
2,2-difluoro-2-(1-methyl-5-(3-(2-methyl-4-(4-(trifluoromethoxy)phenyl)oxazol-5-yl)-3′-(methylsulfonyl)-[1,1′-biphenyl]-4-yl)-1H-pyrazol-3-yl)acetamide
1-(5-(3-(4-(2,2-difluorobenzo[d][1,3]dioxol-5-yl)-2-methyloxazol-5-yl)-3′-(methylsulfonyl)-[1,1′-biphenyl]-4-yl)-1-methyl-1H-pyrazol-3-yl)ethylcarbamate
1-(5-(3-(4-(2,2-difluorobenzo[d][1,3]dioxol-5-yl)-2-methyloxazol-5-yl)-3′-(methylsulfonyl)-[1,1′-biphenyl]-4-yl)-1-methyl-1H-pyrazol-3-yl)ethylcarbamate
1-(5-(3-(4-(2,2-difluorobenzo[d][1,3]dioxol-5-yl)-2-methyloxazol-5-yl)-3′-fluoro-4′-(hydroxymethyl)-5′-(methylsulfonyl)-[1,1′-biphenyl]-4-yl)-1-methyl-1H-pyrazol-3-yl)ethylcarbamate
1-(5-(3-(4-(2,2-difluorobenzo[d][1,3]dioxol-5-yl)-2-methyloxazol-5-yl)-3′-fluoro-4′-(hydroxymethyl)-5′-(methylsulfonyl)-[1,1′-biphenyl]-4-yl)-1-methyl-1H-pyrazol-3-yl)ethylcarbamate
1-(5-(3-(4-(2,2-difluorobenzo[d][1,3]dioxol-5-yl)-2-methyloxazol-5-yl)-3′-fluoro-4′-(hydroxymethyl)-5′-(methylsulfonyl)-[1,1′-biphenyl]-4-yl)-1-methyl-1H-pyrazol-3-yl)ethanone
1-{3′-(methylsulfonyl)-4-[5-methyl-3-(trifluoromethyl)-1H-pyrazol-1-yl]biphenyl-3-yl}-5-{4-[(trifluoromethyl)oxy]phenyl}-1H-1,2,3-triazole
[3-fluoro-5-(methylsulfonyl)-4′-[5-methyl-3-(trifluoromethyl)-1H-pyrazol-1-yl]-3′-(5-{4-[(trifluoromethyl)oxy]phenyl}-1H-1,2,3-triazol-1-yl)biphenyl-4-yl]methanol
[3-chloro-5-(methylsulfonyl)-4′-[5-methyl-3-(trifluoromethyl)-1H-pyrazol-1-yl]-3′-(5-{4-[(trifluoromethyl)oxy]phenyl}-1H-1,2,3-triazol-1-yl)biphenyl-4-yl]methanol
2-methyl-2-{4′-[5-methyl-3-(trifluoromethyl)-1H-pyrazol-1-yl]-3′-(5-{4-[(trifluoromethyl)oxy]phenyl}-1H-1,2,3-triazol-1-yl)biphenyl-3-yl}propanamide
1-{4-[3-(1,1-difluoroethyl)-5-methyl-1H-pyrazol-1-yl]-3′-(methylsulfonyl)biphenyl-3-yl}-5-{4-[(trifluoromethyl)oxy]phenyl}-1H-1,2,3-triazole
{4′-3-(1,1-difluoroethyl)-5-methyl-1H-pyrazol-1-yl]-3-fluoro-5-(methylsulfonyl)-3′-(5-{4-[(trifluoromethyl)oxy]phenyl}-1H-1,2,3-triazol-1-yl)biphenyl-4-} methanol
1-{4-[5-(1,1-difluoroethyl)-3-methyl-1H-pyrazol-1-yl]-3′-(methylsulfonyl)biphenyl-3-yl}-5-{4-[(trifluoromethyl)oxy]phenyl}-1H-1,2,3-triazole
5-{3′-(methylsulfonyl)-4-[5-methyl-3-(trifluoromethyl)-1H-pyrazol-1-yl]biphenyl-3-yl}-4-{4-[(trifluoromethyl)oxy]phenyl}-1,3-oxazole
1-(3-(2-methyl-4-(4-(trifluoromethoxy)phenyl)oxazol-5-yl)-3′-(methylsulfonyl)biphenyl-4-yl)-1H-pyrazole-3-carboxamide
2-methyl-5-(3′-(methylsulfonyl)-4-(3-(trifluoromethyl)-1H-pyrazol-1-yl)-[1,1′-biphenyl]-3-yl)-4-(4-(trifluoromethoxy)phenyl)oxazole
4-(4-(4-chloro-3-(trifluoromethyl)-1H-pyrazol-1-yl)-3′-(methylsulfonyl)-1-1,1′-biphenyl]-3-yl)-2-methyl-5-(4-(trifluoromethoxy)phenyl)oxazole
4-(4-(5-ethoxy-3-(trifluoromethyl)-1H-pyrazol-1-yl)-3′-(methylsulfonyl)biphenyl-3-yl)-2-(trifluoromethoxy)phenyl)oxazole
4-(4-(5-cyclopropyl-3-(trifluoromethyl)-1H-pyrazol-1-yl)-3′-(methylsulfonyl)biphenyl-3-yl)-2-methyl-5-(4-(trifluoromethoxy)phenyl)oxazole
(4′-(3-(1,1-difluoroethyl)-1H-pyrazol-1-yl)-3-fluoro-3′-(2-methyl-4-(4-(trifluoromethoxy)phenyl)oxazol-5-yl)-5-(methylsulfonyl)-[1,1′-biphenyl]-4-yl)methanol
(5-(4-(4-chloro-3-(trifluoromethyl)-1H-pyrazol-1-yl)-3-(2-methyl-4-(4-(trifluoromethoxy)phenyl)oxazol-5-yl)phenyl)-3-(methylsulfonyl)pyridin-2-yl)methanol
(4′-(3-cyclopropyl-1H-pyrazol-1-yl)-3-fluoro-3′-(2-methyl-4-(4-(trifluoromethoxy)phenyl)oxazol-5-yl)-5-(methylsulfonyl)-[1,1′-biphenyl]-4-yl)methanol
2-(1-(3-(2-methyl-4-(4-(trifluoromethoxy)phenyl)oxazol-5-yl)-3′-(methylsulfonyl)biphenyl-4-yl)-1H-pyrazol-3-yl)propan-2-ol
2-(1-(3′-fluoro-4′-(hydroxymethyl)-3-(2-methyl-4-(4-(trifluoromethoxy)phenyl)oxazol-5-yl)-5′-(methylsulfonyl)biphenyl-4-yl)-1H-pyrazol-3-yl)propan-2-ol
1-(3-(2-methyl-4-(4-(trifluoromethoxy)phenyl)oxazol-5-yl)-3′-(methylsulfonyl)biphenyl-4-yl)-1H—H-pyrazole-3-carbonitrile
1-(3′-fluoro-4′-(hydroxymethyl)-3-(2-methyl-4-(4-(trifluoromethoxy)phenyl)oxazol-5-yl)-5′-(methylsulfonyl)biphenyl-4-yl)-1H-pyrazole-3-carbonitrile
1-(3′-fluoro-4′-(hydroxymethyl)-3-(2-methyl-4-(4-(trifluoromethoxy)phenyl)oxazol-5-yl)-5′-(methylsulfonyl)biphenyl-4-yl)-1H-pyrazole-3-carboxamide
(1-(3-(2-methyl-4-(4-(trifluoromethoxy)phenyl)oxazol-5-yl)-3′-(methylsulfonyl)biphenyl-4-yl)-1H-pyrazol-3-yl)methanol
(3-fluoro-4′-(3-(hydroxymethyl)-1H-pyrazol-1-yl)-3′-(2-methyl-4-(4-(trifluoromethoxy)phenyl)oxazol-5-yl)-5-(methylsulfonyl)biphenyl-4-yl)methanol
(4′-(5-cyclopropyl-3-(trifluoromethyl)-1H-pyrazol-1-yl)-3-fluoro-3′-(2-methyl-4-(4-(trifluoromethoxy)phenyl)oxazol-5-yl)-5-(methylsulfonyl)biphenyl-4-yl)methanol
5-(4-(5-cyclopropyl-3-(trifluoromethyl)-1H-pyrazol-1-yl)-3′-(methylsulfonyl)biphenyl-3-yl)-2-methyl-4-(4-(trifluoromethoxy)phenyl)oxazole
(3′-(4-(4-(difluoromethoxy)phenyl)-2-methyloxazol-5-yl)-3-fluoro-4′-(5-methyl-3-(trifluoromethyl)-1H-pyrazol-1-yl)-5-(methylsulfonyl)biphenyl-4-yl)methanol
(3′-(4-(4-(difluoromethoxy)phenyl)-2-methyloxazol-5-yl)-4′-(5-methyl-3-(trifluoromethyl)-1H-pyrazol-1-yl)-3-(methylsulfonyl)biphenyl-4-yl)methanol
4(4-(difluoromethoxy)phenyl)-2-methyl-5-(4-(5-methyl-3-(trifluoromethyl)-1H-pyrazol-1-yl)-3′-methylsulfonyl)biphenyl-3-yl)oxazole
4-(2,2-difluorobenzo[d][1,3]dioxol-5-yl)-2-methyl-5-(4-(5-methyl-3-(trifluoromethyl)-1H-pyrazol-1-yl)-3′-(methylsulfonyl)biphenyl-3-yl)oxazole
(3′-(4-(2,2-difluorobenzo[d][1,3]dioxol-5-yl)-2-methyloxazol-5-yl)-3-fluoro-4′-(5-methyl-3-(trifluoromethyl)-1H-pyrazol-1-yl)-5-(methylsulfonyl)biphenyl-4-yl)methanol difluorobenzo[d][1,3]dioxol-5-yl)-2-methyloxazol-5-yl)-4′(5-methyl-3-(trifluoromethyl)-1H-pyrazol-1-yl)-3-(methylsulfonyl)biphenyl-4-yl)methanol
2-methyl-5-(4-(5-methyl-3-(trifluoromethyl)-1H-pyrazol-1-yl)-3′-(methylsulfonyl)biphenyl-3-yl)-4-(4-(trifluoromethoxy)phenyl)oxazole
(3-fluoro-4′-(5-methyl-3-(trifluoromethyl)-1H-pyrazol-1-yl)-3′-(2-methyl-4-(4-(trifluoromethoxy)phenyl)oxazol-5-yl)-5-(methylsulfonyl)biphenyl-4-yl)methanol
(4′-(5-methyl-3-(trifluoromethyl)-1H-pyrazol-1-yl)-3′-(2-methyl-4-(4-(trifluoromethoxy)phenyl)oxazol-5-yl)-3-(methylsulfonyl)biphenyl-4-yl)methanol
5-(3′-fluoro-5′-(methylsulfonyl)-4-(3-(trifluoromethyl)-1H-pyrazol-1-yl)biphenyl-3-yl)-2-methyl-4-(4-(trifluoromethoxy)phenyl)oxazole
(3-fluoro-3′-(2-methyl-4-(4-(trifluoromethoxy)phenyl)oxazol-5-yl)-5-(methylsulfonyl)-4′-(3-(trifluoromethyl)-1H-pyrazol-1-yl)biphenyl-4-yl)methanol
(3′-(4-(4-chlorophenyl)-2-methyloxazol-5-yl)-3-fluoro-5-(methylsulfonyl)-4′-(3-(trifluoromethyl)-1H-pyrazol-1-yl)biphenyl-4-yl)methanol
4-(2,4-dichlorophenyl)-2-methyl-5-(3′-(methylsulfonyl)-4-(3-(trifluoromethyl)-1H-pyrazol-1-yl)biphenyl-3-yl)oxazole
2-cyclopropyl-5-(3′-(methylsulfonyl)-4-(3-(trifluoromethyl)-1H-pyrazol-1-yl)biphenyl-3-yl)-4-(4-(trifluoromethoxy)phenyl)oxazole
(3′-(2-cyclopropyl-4-(4-(trifluoromethoxy)phenyl)oxazol-5-yl)-3-fluoro-5-(methylsulfonyl)-4′-(3-(trifluoromethyl)-1H-pyrazol-1-yl)biphenyl-4-yl)methanol
2-isopropyl-5-(3′-(methylsulfonyl)-4-(3-(trifluoromethyl)-1H-pyrazol-1-yl)biphenyl-3-yl)-4-(4-(trifluoromethoxy)phenyl)oxazole
(3-fluoro-3′-(2-isopropyl-4-(4-(trifluoromethoxy)phenyl)oxazol-5-yl)-5-(methylsulfonyl)-4′-(3-(trifluoromethyl)-1H-pyrazol-1-yl)biphenyl-4-yl)methanol
2-methyl-2-(3′-(2-methyl-4-(4-(trifluoromethoxy)phenyl)oxazol-5-yl)-4′-(3-(trifluoromethyl)-1H-pyrazol-1-yl)biphenyl-3-yl)propanenitrile
2-methyl-2-(3′-(2-methyl-4-(4-(trifluoromethoxy)phenyl)oxazol-5-yl)-4′-(3-(trifluoromethyl)-1H-pyrazol-1-yl)biphenyl-3-yl)propanamide
4-(4-chlorophenyl)-2-methyl-5-(3′-(methylsulfonyl)-4-(3-(trifluoromethyl)-1H-pyrazol-1-yl)biphenyl-3-yl)oxazole
5-(4-(5-chloro-3-(trifluoromethyl)-1H-pyrazol-1-yl)-3′-(methylsulfonyl)biphenyl-3-yl)-2-methyl-4-(4-(trifluoromethoxy)phenyl)oxazole
5-(4-(5-chloro-3-(trifluoromethyl)-1H-pyrazol-1-yl)-3′-(methylsulfonyl)biphenyl-3-yl)-4-(2,2-difluorobenzo[d][1,3]dioxol-5-yl)-2-methyloxazole
(4′-(5-chloro-3-(trifluoromethyl)-1H-pyrazol-1-yl)-3′-(4-(2,2-(4-difluorobenzo[d][1,3]dioxol-5-yl)-2-methyloxazol-5-yl)-3-fluoro-5-(methylsulfonyl)biphenyl-4-yl)methanol
4′45-chloro-3-(trifluoromethyl)-1H-pyrazol-1-yl)-3-fluoro-3′42-methyl-4-(4-(trifluoromethoxy)phenyl)oxazol-5-yl)-5-(methylsulfonyl)biphenyl-4-yl)methanol
(3′(4-(2,2-difluorobenzo[d][1,3]dioxol-5-yl)-2-methyloxazol-5-yl)-3-fluoro-5-(methylsulfonyl)-4′-(3-(trifluoromethyl)-1H-pyrazol-1-yl)biphenyl-4-yl)methanol
4-(2,2-difluorobenzo[d][1,3]dioxol-5-yl)-2-methyl-5-(3′-(methylsulfonyl)-4-(3-(trifluoromethyl)-1H-pyrazol-1-yl)biphenyl-3-yl)oxazole
4-(4-(difluoromethoxy)-3,5-difluorophenyl)-2-methyl-5-(3′-(methylsulfonyl)-4-(3-(trifluoromethyl)-1H-pyrazol-1-yl)biphenyl-3-yl)oxazole
(3′-(4-(4-(difluoromethoxy)-3,5-difluorophenyl)-2-methyloxazol-5-yl)-3-fluoro-5-(methylsulfonyl)-4′-(3-(trifluoromethyl)-1H-pyrazol-1-yl)biphenyl-4-yl)methanol
4-(6-(difluoromethoxy)pyridin-3-yl)-2-methyl-5-(3′-(methylsulfonyl)-4-(3-(trifluoromethyl)-1H-pyrazol-1-yl)biphenyl-3-yl)oxazole
(3′-(4-(6-(difluoromethoxy)pyridin-3-yl)-2-methyloxaz01-5-yl)-3-fluoro-5-(methylsulfonyl)-4′-(3-(trifluoromethyl)-1H-pyrazol-1-yl)biphenyl-4-yl)methanol
4-(4-chloro-3-(difluoromethoxy)phenyl)-2-methyl-5-(3′-(methylsulfonyl)-4-(3-(trifluoromethyl)-1H-pyrazol-1-yl)biphenyl-3-yl)oxazole
(3′-(4-(4-chloro-3-(difluoromethoxy)phenyl)-2-methyloxazol-5-yl)-3-fluoro-5-(methylsulfonyl)-4′-(3-(trifluoromethyl)-1H-pyrazol-1-yl)biphenyl-4-yl)methanol
2-(1-(3-(4-(2,2-difluorobenzo[d][1,3]dioxol-5-yl)-2-methyloxazol-5-yl)-3′-(methylsulfonyl)-[1,1′-biphenyl]-4-yl)-1H-pyrazol-3-yl)propan-2-ol
2-(1-(3-(4-(2,2-difluorobenzo[d][1,3]dioxol-5-yl)-2-methyloxazol-5-yl)-3′-fluoro-4′-(hydroxymethyl)-5′-(methylsulfonyl)-[1,1′-biphenyl]-4-yl)-1H-pyrazol-3-yl)propan-2-ol
1-(3-(4-(2,2-difluorobenzo[d][1,3]dioxol-5-yl)-2-methyloxazol-5-yl)-3′-(methylsulfonyl)-[1,1′-biphenyl]-4-yl)-1H-pyrazole-3-carboxamide
1-(3-(4-(2,2-difluorobenzo[d][1,3]dioxol-5-yl)-2-methyloxazol-5-yl)-3′-fluoro-4′-(hydroxymethyl)-5′-(methylsulfonyl)-[1,1′-biphenyl]-4-yl)-1H-pyrazole-3-carboxamide
1-(3-(2-cyclopropyl-4-(4-(trifluoromethoxy)phenyl)oxazol-5-yl)-3′-(methylsulfonyl)[1,1′-biphenyl]-4-yl)-1H-pyrazole-3-carboxamide
1-(3-(4-(2,2-difluorobenzo[d][1,3]dioxol-5-yl)-2-methyloxazol-5-yl)-3′-(methylsulfonyl)-[1,1′-biphenyl]-4-yl)-1H-pyrazole-3-carbonitrile
(1-(3-(4-(2,2-difluorobenzo[d][1,3]dioxol-5-yl)-2-methyloxazol-5-yl)-3′-(methylsulfonyl)-[1,1′-biphenyl]-4-yl)-1H-pyrazol-3-yl)methanol
1-(3′-(1-amino-2-methyl-1-oxopropan-difluorobenzo[d][1,3]dioxol-5-yl)-2-methyloxazol-5-yl)-[1,1′-biphenyl]-4-yl)-1H-pyrazole-3-carboxamide
2-(4′-(3-(cyanomethyl)-1H-pyrazol-1-yl)-3′-(2-methyl-4-(4-(trifluoromethoxy)phenyl)oxazol-5-yl)-[1,1′-biphenyl]-3-yl)-2-methylpropanamide
1-(3′-(1-amino-2-methyl-1-oxopropan-2-yl)-3-(2-methyl-4-(4-(trifluoromethoxy)phenyl)oxazol-5-yl)-[1,1′-biphenyl]-4-yl)-N-(2,2,2-trifluoroethyl)-1H-pyrazole-3-carboxamide
1-(1-(3-(2-methyl-4-(4-(trifluoromethoxy)phenyl)oxazol-5-yl)-3′-(methylsulfonyl)[1,1′-biphenyl]-4-yl)-1H-pyrazol-3-yl)ethanol
1-(1-(3-(2-methyl-4-(4-(trifluoromethoxy)phenyl)oxazol-5-yl)-3′-(methylsulfonyl)[1,1′-biphenyl]-4-yl)-1H-pyrazol-3-yl)ethanol
2-(1-(3-(2-methyl-4-(4-(trifluoromethoxy)phenyl)oxazol-5-yl)-3′-(methylsulfonyl)-[1,1′-biphenyl]-4-yl)-1H-pyrazol-3-yl)acetonitrile
2-(1-(3′-fluoro-4′-(hydroxymethyl)-3-(2-methyl-4-(4-(trifluoromethoxy)phenyl)oxazol-5-yl)-5′-(methylsulfonyl)-[1,1′-biphenyl]-4-yl)-1H-pyrazol-3-yl)acetonitrile
1-(3-(2-methyl-4-(4-(trifluoromethoxy)phenyl)oxazol-5-yl)-3′-(methylsulfonyl)-[1,1′-biphenyl]-4-yl)-N-(2,2,2-trifluoroethyl)-1H-pyrazole-3-carboxamide
1-(3′-fluoro-4′-(hydroxymethyl)-3-(2-methyl-4-(4-(trifluoromethoxy)phenyl)oxazol-5-yl)-5′-(methylsulfonyl)-[1,1′-biphenyl]-4-yl)-N-(2,2,2-trifluoroethyl)-1H-pyrazole-3-carboxamide
2-methyl-2-(1-(3-(2-methyl-4-(4-(trifluoromethoxy)phenyl)oxazol-5-yl)-3′-(methylsulfonyl)-[1,1′-biphenyl]-4-yl)-1H-pyrazol-3-yl)propanenitrile
(1-(3-(2-methyl-4-(4-(trifluoromethoxy)phenyl)oxazol-5-yl)-3′-(methylsulfonyl)-[1,1′-biphenyl]-4-yl)-1H-pyrazol-3-yl)methylcarbamate
(1-(3′-fluoro-4′-(hydroxymethyl)-3-(2-methyl-4-(4-(trifluoromethoxy)phenyl)oxazol-5-yl)-5′-(methylsulfonyl)-[1,1′-biphenyl]-4-yl)-1H-pyrazol-3-yl)methylcarbamate
(1-(3′-(1-amino-2-methyl-1-oxopropan-2-yl)-3-(2-methyl-4-(4-(trifluoromethoxy)phenyl)oxazol-5-yl)-[1,1′-biphenyl]-4-yl)-1H-pyrazol-3-yl)methylcarbamate
2-(1-(3′-fluoro-4′-(hydroxymethyl)-3-2-methyl-4-(4-(trifluoromethoxy)phenyl)oxazol-5-yl)-5′-(methylsulfonyl)-[1,1′-biphenyl]-4-yl)-1H-pyrazol-3-yl)-2-methylpropanenitrile
2-(1-(4′-(hydroxymethyl)-3-(2-methyl-4-(4-(trifluoromethoxy)phenyl)oxazol-5-yl)-3′-(methylsulfonyl)-[1,1′-biphenyl]-4-yl)-1H-pyrazol-3-yl)-2-methylpropanenitrile
(4′-(4-chloro-3-(trifluoromethyl)-1H-pyrazol-1-yl)-3-fluoro-3′-(2-methyl-4-(4-(trifluoromethoxy)phenyl)oxazol-5-yl)-5-(methylsulfonyl)-[1,1′-biphenyl]-4-yl)methanol
5-(4-(4-chloro-3-(trifluoromethyl)-1H-pyrazol-1-yl)-3′-(methylsulfonyl)-[1,1′-biphenyl]-3-yl)-2-methyl-4-(4-(trifluoromethoxy)phenyl)oxazole
1-(4′-(5-chloro-3-(trifluoromethyl)-1H-pyrazol-1-yl)-3′-(2-methyl-4-(4-(trifluoromethoxy)phenyl)oxazol-5-yl)-[1,1′-biphenyl]-3-yl)cyclopropanecarboxamide
(4′-(5-ethoxy-3-(trifluoromethyl)-1H-pyrazol-1-yl)-3-fluoro-3′-(2-methyl-5-(4-(trifluoromethoxy)phenyl)oxazol-4-yl)-5-(methylsulfonyl)biphenyl-4-yl)methanol
5-(4-(difluoromethoxy)phenyl)-2-methyl-4-(4-(5-methyl-3-(trifluoromethyl)-1H-pyrazol-1-yl)-3′-(methylsulfonyl)biphenyl-3-yl)oxazole
(3′-(5-(4-(difluoromethoxy)phenyl)-2-methyloxazol-4-yl)-3-fluoro-4′-(5-methyl-3-(trifluoromethyl)-1H-pyrazol-1-yl)-5-(methylsulfonyl)biphenyl-4-yl)methanol
2-methyl-4-(4-(5-methyl-3-(trifluoromethyl)-1H-pyrazol-1-yl)-3′-(methylsulfonyl)biphenyl-3-yl)-5-(4-(trifluoromethoxy)phenyl)oxazole
(3-fluoro-4′-(5-methyl-3-(trifluoromethyl)-1H-pyrazol-1-yl)-3′-(2-methyl-5-(4-(trifluoromethoxy)phenyl)oxazol-4-yl)-5-(methylsulfonyl)biphenyl-4-yl)methanol
(4′-(5-methyl-3-(trifluoromethyl)-1H-pyrazol-1-yl)-3′-(2-methyl-5-(4-(trifluoromethoxy)phenyl)oxazol-4-yl)-3-(methylsulfonyl)biphenyl-4-yl)methanol
5-(2,2-difluorobenzo[d][1,3]dioxol-5-yl)-2-methyl-4-(4-(5-methyl-3-(trifluoromethyl)-1H-pyrazol-1-yl)-3′-(methylsulfonyl)biphenyl-3-yl)oxazole difluorobenzo[d][1,3]dioxol-5-yl)-2-methyloxazol-4-yl)-3-fluoro-4′-(5-methyl-3-(trifluoromethyl)-1H-pyrazol-1-yl)-5-(methylsulfonyl)biphenyl-4-yl)methanol
(3′-(5-(2,2-difluorobenzo[d][1,3]dioxol-5-yl)-2-methyloxazol-4-yl)-4′-(5-methyl-3-(trifluoromethyl)-1H-pyrazol-1-yl)-3-(methylsulfonyl)biphenyl-4-yl)methanol
(3′-(5-(4-(difluoromethoxy)phenyl)-2-methyloxazol-4-yl)-3-fluoro-4′-(3-methyl-5-(trifluoromethyl)-1H-pyrazol-1-yl)-5-(methylsulfonyl)biphenyl-4-yl)methanol
(3′-(5-(4-(difluoromethoxy)phenyl)-2-methyloxazol-4-yl)-4′-(5-methyl-3-(trifluoromethyl)-1H-pyrazol-1-yl)-3-methylsulfonyl)biphenyl-4-yl)methanol
5-(4-(difluoromethoxy)phenyl)-2-methyl-4-(4-(3-methyl-5-(trifluoromethyl)-1H-pyrazol-1-yl)-3′-(methylsulfonyl)biphenyl-3-yl)oxazole
5-(6-(difluoromethoxy)pyridin-3-yl)-2-methyl-4-(3′-(methylsulfonyl)-4-(3-(trifluoromethyl)-1H-pyrazol-1-yl)biphenyl-3-yl)oxazole
(3′-(5-(6-(difluoromethoxy)pyridin-3-yl)-2-methyloxazol-4-yl)-3-fluoro-5-(methylsulfonyl)-4′-(3-(trifluoromethyl)-1H-pyrazol-1-yl)biphenyl-4-yl)methanol
(3′-(5-(4-chloro-3-(difluoromethoxy)phenyl)-2-methyloxazol-4-yl)-3-fluoro-5-(methylsulfonyl)-4′-(3-(trifluoromethyl)-1H-pyrazol-1-yl)biphenyl-4-yl)methanol
5-(3-chloro-4-(difluoromethoxy)phenyl)-2-methyl-4-(3′-(methylsulfonyl)-4-(3-(trifluoromethyl)-1H-pyrazol-1-yl)biphenyl-3-yl)oxazole
(3-fluoro-3′-(2-isopropyl-5-(4-(trifluoromethoxy)phenyl)oxazol-4-yl)-5-(methylsulfonyl)-4′-(3-(trifluoromethyl)-1H-pyrazol-1-yl)biphenyl-4-yl)methanol
2-isopropyl-4-(3′-(methylsulfonyl)-4-(3-(trifluoromethyl)-1H-pyrazol-1-yl)biphenyl-3-yl)-5-(4-(trifluoromethoxy)phenyl)oxazole
(3′-(5-(4-chlorophenyl)-2-methyloxazol-4-yl)-3-fluoro-5-(methylsulfonyl)-4′-(3-(trifluoromethyl)-1H-pyrazol-1-yl)biphenyl-4-yl)methanol
5-(4-chlorophenyl)-2-methyl-4-(3′-(methylsulfonyl)-4-(3-(trifluoromethyl)-1H-pyrazol-1-yl)biphenyl-3-yl)oxazole
(3′-(2-cyclopropyl-5-(4-(trifluoromethoxy)phenyl)oxazol-4-yl)-3-fluoro-5-(methylsulfonyl)-4′-(3-(trifluoromethyl)-1H-pyrazol-1-yl)biphenyl-4-yl)methanol
2-cyclopropyl-4-(3′-(methylsulfonyl)-4-(3-(trifluoromethyl)-1H-pyrazol-1-yl)biphenyl-3-yl)-5-(4-(trifluoromethoxy)phenyl)oxazole
2-(4′-(5-chloro-3-(trifluoromethyl)-1H-pyrazol-1-yl)-3′-(2-methyl-5-(4-(trifluoromethoxy)phenyl)oxazol-4-yl)biphenyl-3-yl)-2-methylpropanamide
(4′-(5-chloro-3-(trifluoromethyl)-1H-pyrazol-1-yl)-3-fluoro-3′-(2-methyl-5-(4-(trifluoromethoxy)phenyl)oxazol-4-yl)-5-(methylsulfonyl)biphenyl-4-yl)methanol
4-(4-(5-chloro-3-(trifluoromethyl)-1H-pyrazol-1-yl)-3′-(methylsulfonyl)biphenyl-3-yl)-2-methyl-5-(4-(trifluoromethoxy)phenyl)oxazole
5-(5-fluoro-6-methoxypyridin-3-yl)-2-methyl-4-(3′-(methylsulfonyl)-4-(3-(trifluoromethyl)-1H-pyrazol-1-yl)biphenyl-3-yl)oxazole
(3-fluoro-3′-(5-(5-fluoro-6-methoxypyridin-3-yl)-2-methyloxazol-4-yl)-5-(methylsulfonyl)-4′-(3-(trifluoromethyl)-1H-pyrazol-1-yl)biphenyl-4-yl)methanol
5-(4-(difluoromethoxy)-3,5-difluorophenyl)-2-methyl-4-(3′-(methylsulfonyl)-4-(3-(trifluoromethyl)-1H-pyrazol-1-yl)biphenyl-3-yl)oxazole
(3′-(5-(4-(difluoromethoxy)-3,5-difluorophenyl)-2-methyloxazol-4-yl)-3-fluoro-5-(methylsulfonyl)-4′-(3-(trifluoromethyl)-1H-pyrazol-1-yl)biphenyl-4-yl)methanol
(4′-(5-chloro-3-(trifluoromethyl)-1H-pyrazol-1-yl)-3′-(5-(2,2-difluorobenzo[d][1,3]dioxol-5-yl)-2-methyloxazol-4-yl)-3-fluoro-5-(methylsulfonyl)biphenyl-4-yl)methanol
4-(4-(5-chloro-3-(trifluoromethyl)-1H-pyrazol-1-yl)-3′-(methylsulfonyl)biphenyl-3-yl)-5-(2,2-difluorobenzo[d][1,3]dioxol-5-yl)-2-methyloxazole
(3-fluoro-3′-(2-methyl-5-(4-trifluoromethoxy)phenyl)oxazol-4-yl)-5-(methylsulfonyl)-4′-(3-(trifluoromethyl)-1H-pyrazol-1-yl)biphenyl-4-yl)methanol
2-methyl-4-(3′-(methylsulfonyl)-4-(3-(trifluoromethyl)-1H-pyrazol-1-yl)biphenyl-3-yl)-5-(4-(trifluoromethoxy)phenyl)oxazole
(4′-(5-cyclopropyl-3-(trifluoromethyl)-1H-pyrazol-1-yl)-3-fluoro-3′-(2-methyl-5-(4-(trifluoromethoxy)phenyl)oxazol-4-yl)-5-(methylsulfonyl)biphenyl-4-yl)methanol
1-(3-(2-methyl-5-(4-(trifluoromethoxy)phenyl)oxazol-4-yl)-3′-(methylsulfonyl)-[1,1′-biphenyl]-4-yl)-1H-pyrazole-3-carboxamide
2-(1-(3-(2-methyl-5-(4-(trifluoromethoxy)phenyl)oxazol-4-yl)-3′-(methylsulfonyl)41,1′-biphenyl]-4-yl)-1H-pyrazol-3-yl)propan-2-ol
1-(4′-(5-chloro-3-(trifluoromethyl)-1H-pyrazol-1-yl)-3′-(2-methyl-5-(4-(trifluoromethoxy)phenyl)oxazol-4-yl)-[1,1′-biphenyl]-3-yl)cyclopropanecarboxamide
5-(4-(4-chloro-3-(trifluoromethyl)-1H-pyrazol-1-yl)-3′-(methylsulfonyl)-[1,1′-biphenyl]-3-yl)-4-(2,2-difluorobenzo[d][1,3]dioxol-5-yl)-2-methyloxazole
(4′-(4-chloro-3-(trifluoromethyl)-1H-pyrazol-1-yl)-3′-(4-(2,2-difluorobenzo[d][1,3]dioxol-5-yl)-2-methyloxazol-5-yl)-3-fluoro-5-(methylsulfonyl)-[1,1′-biphenyl]-4-yl)methanol
5-(4-(4-chloro-3-(trifluoromethyl)-1H-pyrazol-1-yl)-3′-(methylsulfonyl)-[1,1′-biphenyl]-3-yl)-4-(4-(difluoromethoxy)-3,5-difluorophenyl)-2-methyloxazole
(4′-(4-chloro-3-(trifluoromethyl)-1H-pyrazol-1-yl)-3′(4-(4-(difluoromethoxy)-3,5-difluorophenyl)-2-methyloxazol-5-yl)-3-fluoro-5-(methylsulfonyl)-[1,1′-biphenyl]-4-yl)methanol
5-(4-(4-chloro-3-(trifluoromethyl)-1H-pyrazol-1-yl)-3′-(methylsulfonyl)-[1,1′-biphenyl]-3-yl)-4-(2,4-dichlorophenyl)-2-methyloxazole
(4′-(4-chloro-3-trifluoromethyl)-1H-pyrazol-1-yl)-3′-(4-(2,4-(4dichlorophenyl)-2-methyloxazol-5-yl)-3-fluoro-5-(methylsulfonyl)-[1,1′-biphenyl]-4-yl)methanol
5-(4-(4-chloro-3-(trifluoromethyl)-1H-pyrazol-1-yl)-3′-(methylsulfonyl)-[1,1′-biphenyl]-3-yl)-4-(6-(difluoromethoxy)pyridin-3-yl)-2-methyloxazole
(4′-(4-chloro-3-(trifluoromethyl)-1H-pyrazol-1-yl)-3′-(4-(6-(difluoromethoxy)pyridin-3-yl)-2-methyloxazol-5-yl)-3-fluoro-5-(methylsulfonyl)-[1,1′-biphenyl]-4-yl)methanol
5-(4-(4-chloro-3-(trifluoromethyl)-1H-pyrazol-1-yl)-3′-(methylsulfonyl)-[1,1′-biphenyl]-3-yl)-4-(2-methoxypyrimidin-5-yl)-2-methyloxazole
(4′-(4-chloro-3-(trifluoromethyl)-1H-pyrazol-1-yl)-3-fluoro-3′-(4-(2-methoxypyrimidin-5-yl)-2-methyloxazol-5-yl)-5-(methylsulfonyl)-[1,1′-biphenyl]-4-yl)methanol
2-(4′-(4-chloro-3-(trifluoromethyl)-1H-pyrazol-1-yl)-3′-(2-methyl-4-(4-(trifluoromethoxy)phenyl)oxazol-5-yl)-[1,1′-biphenyl]-3-yl)-2-methylpropanenitrile
2-(4′-(4-chloro-3-(trifluoromethyl)-1H-pyrazol-1-yl)-3′-(2-methyl-4-(4-(trifluoromethoxy)phenyl)oxazol-5-yl)-[1,1′-biphenyl]-3-yl)-2-methylpropanamide
5-(4-(4-chloro-3-(trifluoromethyl)-1H-pyrazol-1-yl)-3′-(methylsulfonyl)-[1,1′-biphenyl]-3-yl)-4-(2-chloro-4-(difluoromethoxy)phenyl)-2-methyloxazole
(4′-(4-chloro-3-(trifluoromethyl)-1H-pyrazol-1-yl)-3′-(4-(2-chloro-4-(difluoromethoxy)phenyl)-2-methyloxazol-5-yl)-3-fluoro-5-(methylsulfonyl)-[1,1′-biphenyl]-4-yl)methanol
5-(4-(4-chloro-3-(trifluoromethyl)-1H-pyrazol-1-yl)-3′-(methylsulfonyl)-[1,1′-biphenyl]-3-yl)-4-(4-(difluoromethoxy)-3-fluorophenyl)-2-methyloxazole
(4′-(4-chloro-3-(trifluoromethyl)-1H-pyrazol-1-yl)-3′-(4-(4-(difluoromethoxy)-3-fluorophenyl)-2-methyloxazol-5-yl)-3-fluoro-5-(methylsulfonyl)-[1,1′-biphenyl]-4-yl)methanol
4-(4-(difluoromethoxy)phenyl)-2-methyl-5-(3′-(methylsulfonyl)-4-(3-(trifluoromethyl)-1H-pyrazol-1-yl)-[1,1′-biphenyl]-3-yl)oxazole
(3′-(4-(4-(difluoromethoxy)phenyl)-2-methyloxazol-5-yl)-3-fluoro-5-(methylsulfonyl)-4′-(3-(trifluoromethyl)-1H-pyrazol-1-yl)-[1,1′-biphenyl]-4-yl)methanol
5-(4-(4-chloro-3-(trifluoromethyl)-1H-pyrazol-1-yl)-3′-(methylsulfonyl)-[1,1-biphenyl]-3-yl)-2-methyl-4-(4-(trifluoromethyl)phenyl)oxazole
(4′-(4-chloro-3-(trifluoromethyl)-1H-pyrazol-1-yl)-3-fluoro-3′-(2-methyl-4-(4-(trifluoromethyl)phenyl)oxazol-5-yl)-5-(methylsulfonyl)-[1,1′-biphenyl]-4-yl)methanol
(3-fluoro-4′-(4-methyl-3-(trifluoromethyl)-1H-pyrazol-1-yl)-3′-(2-methyl-4-(4-(trifluoromethoxy)phenyl)oxazol-5-yl)-5-(methylsulfonyl)-[1,1′-biphenyl]-4-yl)methanol
2-methyl-5-(4-(4-methyl-3-(trifluoromethyl)-1H-pyrazol-1-yl)-3′-(methylsulfonyl)-[1,1′-biphenyl]-3-yl)-4-(4-(trifluoromethoxy)phenyl)oxazole
2-(4-chloro-1-(3-(2-methyl-4-(4-(trifluoromethoxy)phenyl)oxazol-5-yl)-3′-(methylsulfonyl)41,1′-biphenyl]-4-yl)-1H-pyrazol-3-yl)propan-2-ol
5-(4-(3-(1,1-difluoroethyl)-1H-pyrazol-1-yl)-3′-(methylsulfonyl)-[1,1′-biphenyl]-3-yl)-2-methyl-4-(4-(trifluoromethoxy)phenyl)oxazole
2-(4-chloro-1-(3′-fluoro-4′-(hydroxymethyl)-3-(2-methyl-4-(4-(trifluoromethoxy)phenyl)oxazol-5-yl)-5′-(methylsulfonyl)-[1,1′-biphenyl]-4-yl)-1H-pyrazol-3-yl)propan-2-ol
4′-(4-chloro-3-(trifluoromethyl)-1H-pyrazol-1-yl)-N-methyl-3′-(2-methyl-4-(4-(trifluoromethoxy)phenyl)oxazol-5-yl)-[1,1′-biphenyl]-3-sulfonamide
4′-(4-chloro-3-(trifluoromethyl)-1H-pyrazol-1-yl)-3′-(4-(2-methoxypyrimidin-5-yl)-2-methyloxazol-5-yl)-N-methyl-[1,1′-biphenyl]-3-sulfonamide
3-cyclopropyl-1-(3′-fluoro-4′-(hydroxymethyl)-3-(2-methyl-4-(4-(trifluoromethoxy)phenyl)oxazol-5-yl)-5′-(methylsulfonyl)-[1,1′-biphenyl]-4-yl)-1H-pyrazole-4-carbonitrile
3-cyclopropyl-1-(3-(2-methyl-4-(4-(trifluoromethoxy)phenyl)oxazol-5-yl)-3′-(methylsulfonyl)41,1′-biphenyl]-4-yl)-1H-pyrazole-4-carbonitrile
3-cyclopropyl-1-(3′-fluoro-4′-(hydroxymethyl)-3-(2-methyl-4-(4-(trifluoromethoxy)phenyl)oxazol-5-yl)-5′-(methylsulfonyl)41,1′-biphenyl]-4-yl)-1H-pyrazole-4-carboxamide
   or a pharmaceutically acceptable salt thereof.

In another aspect, the compound is a compound described in U.S. Pat. No. 9,834,542 (see the formula below).

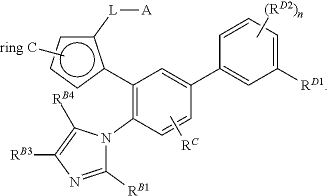

In another aspect, the compound is selected from:
{4′-[5-chloro-2-methyl-4-(trifluoromethyl)-1H-imidazol-1-yl]-3-fluoro-5-(methylsulfonyl)-3′-(5-{4-[(trifluoromethyl)oxy]phenyl}-1H-1,2,3-triazol-1-yl)biphenyl-4-yl}methanol;
5-(4-chlorophenyl)-1-{4-[2-chloro-4-(trifluoromethyl)-1H-imidazol-1-yl]-3′-(methylsulfonyl)biphenyl-3-yl}-1H-1,2,3-triazole;
2-methyl-1-[3′-(methylsulfonyl)-3-(5-{4-[(trifluoromethyl)oxy]phenyl}-1H-1,2,3-triazol-1-yl)biphenyl-4-yl]-N-(2,2,2-trifluoroethyl)-1H-imidazole-4-carboxamide;
5-[4-(fluoromethyl)phenyl]-1-{3′-(methylsulfonyl)-4-[2-methyl-4-(trifluoromethyl)-1H-imidazol-1-yl]biphenyl-3-yl}-1H-1,2,3-triazole;
5-[4-(difluoromethyl)phenyl]-1-{3′-(methylsulfonyl)-4-[2-methyl-4-(trifluoromethyl)-1H-imidazol-1-yl]biphenyl-3-yl}-1H-1,2,3-triazole;
1-(4-(4-(difluoromethyl)-2-methyl-1H-imidazol-1-yl)-3′-(methylsulfonyl)-[1,1′-biphenyl]-3-yl)-5-(4-(trifluoromethoxy)phenyl)-1H-1,2,3-triazole;
1-(5-chloro-3′-(methylsulfonyl)-4-(4-(trifluoromethyl)-1H-imidazol-1-yl)biphenyl-3-yl)-5-(4-chlorophenyl)-1H-1,2,3-triazole;
5-(2,4-dichlorophenyl)-1-{5-fluoro-3′-(methylsulfonyl)-4-[2-methyl-4-(trifluoromethyl)-1H-imidazol-1-yl]biphenyl-3-yl}-1H-1,2,3-triazole;
(3-fluoro-3′-{5-[1-(4-methylphenyl)ethyl]-1H-1,2,3-triazol-1-yl}-5-(methylsulfonyl)-4′-[2-methyl-4-(trifluoromethyl)-1H-imidazol-1-yl]biphenyl-4-yl)methanol;
(4-chlorophenyl)(1-{3′-(methylsulfonyl)-4-[2-methyl-4-(trifluoromethyl)-1H-imidazol-1-yl]biphenyl-3-yl}-1H-1,2,3-triazol-5-yl)methanone;
1-(4-chlorophenyl)-2,2,2-trifluoro-1-(1-{3′-(methylsulfonyl)-4-[2-methyl-4-(trifluoromethyl)-1H-imidazol-1-yl]biphenyl-3-yl}-1H-1,2,3-triazol-5-yl)ethanol;
5-[(4-chlorophenyl)(difluoro)methyl]-1-{3′-(methylsulfonyl)-4-[2-methyl-4-(trifluoromethyl)-1H-imidazol-1-yl]biphenyl-3-yl}-1H-1,2,3-triazole;
5-(4-chlorophenyl)-1-{3′-(methylsulfonyl)-4-[4-(trifluoromethyl)-1H-imidazol-1-yl]biphenyl-3-yl}-1H-1,2,3-triazole;
{3-chloro-3′-[5-(2,4-dichlorophenyl)-1H-1,2,3-triazol-1-yl]-5-(methylsulfonyl)-4′-[2-methyl-4-(trifluoromethyl)-1H-imidazol-1-yl]biphenyl-4-yl}methanol;
5-(2,4-dichlorophenyl)-1-{3′-(methylsulfonyl)-4-[4-(trifluoromethyl)-1H-imidazol-1-yl]biphenyl-3-yl}-1H-1,2,3-triazole;
1-{3′-(methylsulfonyl)-4-[2-methyl-4-(trifluoromethyl)-1H-imidazol-1-yl]biphenyl-3-yl}-5-{3-methyl-4-[(trifluoromethyl)oxy]phenyl}-1H-1,2,3-triazole;
{3-chloro-3′-[5-(2,2-difluoro-1,3-benzodioxol-5-yl)-1H-1,2,3-triazol-1-yl]-5-(methylsulfonyl)-4′-[2-methyl-4-(trifluoromethyl)-1H-imidazol-1-yl]biphenyl-4-yl}methanol;
N,N-dimethyl-4-(1-{3′-(methylsulfonyl)-4-[2-methyl-4-(trifluoromethyl)-1H-imidazol-1-yl]biphenyl-3-yl}-1H-1,2,3-triazol-5-yl)aniline;
1-{3′-(methylsulfonyl)-4-[2-methyl-4-(trifluoromethyl)-1H-imidazol-1-yl]biphenyl-3-yl}-5-{4-[(1,1,2,2-tetrafluoroethyl)oxy]phenyl}-1H-1,2,3-triazole;
5-[2-methyl-4-(methyloxy)phenyl]-1-{3′-(methylsulfonyl)-4-[2-methyl-4-(trifluoromethyl)-1H-imidazol-1-yl]biphenyl-3-yl}-1H-1,2,3-triazole;
1-{3′-(methylsulfonyl)-4-[2-methyl-4-trifluoromethyl)-1H-imidazol-1-yl]biphenyl-3-yl}-5-[3-(trifluoromethyl)phenyl]-1H-1,2,3-triazole;
5-[4-(methyloxy)phenyl]-1-{3′-(methylsulfonyl)-4-[2-methyl-4-(trifluoromethyl)-1H-imidazol-1-yl]biphenyl-3-yl}-1H-1,2,3-triazole;
2-methyl-2-{4′-[2-methyl-4-(trifluoromethyl)-1H-imidazol-1-yl]-3′-(5-{4-[(trifluoromethyl)oxy]phenyl}-1H-1,2,3-triazol-1-yl)biphenyl-3-yl}propanamide;
5-(4-fluoro-3-methylphenyl)-1-{3′-(methylsulfonyl)-4-[2-methyl-4-(trifluoromethyl)-1H-imidazol-1-yl]biphenyl-3-yl}-1H-1,2,3-triazole;
5-(1,3-benzodioxol-5-yl)-1-{3′-(methylsulfonyl)-4-[2-methyl-4-(trifluoromethyl)-1H-imidazol-1-yl]biphenyl-3-yl}-1H-1,2,3-triazole;
5-[4-(butyloxy)phenyl]-1-{3′-(methylsulfonyl)-4-[2-methyl-4-(trifluoromethyl)-1H-imidazol-1-yl]biphenyl-3-yl}-1H-1,2,3-triazole;
1-{3′-(methylsulfonyl)-4-[2-methyl-4-(trifluoromethyl)-1H-imidazol-1-yl]biphenyl-3-yl}-5-{4-[(2,2,2-trifluoroethyl)oxy]phenyl}-1H-1,2,3-triazole;
1-{3′-(methylsulfonyl)-4-[2-methyl-4-(trifluoromethyl)-1H-imidazol-1-yl]biphenyl-3-yl}-5-{4-[(trifluoromethyl)thio]phenyl}-1H-1,2,3-triazole;
5-(3-chloro-4-fluorophenyl)-1-{3′-(methylsulfonyl)-4-[2-methyl-4-(trifluoromethyl)-1H-imidazol-1-yl]biphenyl-3-yl}-1H-1,2,3-triazole;
5-(3,4-difluorophenyl)-1-{3′-(methylsulfonyl)-4-[2-methyl-4-(trifluoromethyl)-1H-imidazol-1-yl]biphenyl-3-yl}-1H-1,2,3-triazole;
5-{4-chloro-3-[(trifluoromethyl)oxy]phenyl}-1-{3′-(methylsulfonyl)-4-[2-methyl-4-(trifluoromethyl)-1H-imidazol-1-yl]biphenyl-3-yl}-1H-1,2,3-triazole;
1-[4-(1-{3′-(methylsulfonyl)-4-[2-methyl-4-(trifluoromethyl)-1H-imidazol-1-yl]biphenyl-3-yl}-1H-1,2,3-triazol-5-yl)phenyl]ethanone;
5-{3-[(difluoromethyl)oxy]phenyl}-1-{3′-(methylsulfonyl)-4-[2-methyl-4-(trifluoromethyl)-1H-imidazol-1-yl]biphenyl-3-yl}-1H-1,2,3-triazole;
5-(4-chloro-2-fluorophenyl)-1-{3′-(methylsulfonyl)-4-[2-methyl-4-(trifluoromethyl)-1H-imidazol-1-yl]biphenyl-3-yl}-1H-1,2,3-triazole;
5-(2,4-difluorophenyl)-1-{3′-(methylsulfonyl)-4-[2-methyl-4-(trifluoromethyl)-1H-imidazol-1-yl]biphenyl-3-yl}-1H-1,2,3-triazole;
2-chloro-5-(1-{3′-(methylsulfonyl)-4-[2-methyl-4-(trifluoromethyl)-1H-imidazol-1-yl]biphenyl-3-yl}-1H-1,2,3-triazol-5-yl)benzonitrile;
5-{3-[(difluoromethyl)oxy]-4-(methyloxy)phenyl}-1-{3′-(methylsulfonyl)-4-[2-methyl-4-(trifluoromethyl)-1H-imidazol-1-yl]biphenyl-3-yl}-1H-1,2,3-triazole;
5-(4-fluorophenyl)-1-{3′-(methylsulfonyl)-4-[2-methyl-4-(trifluoromethyl)-1H-imidazol-1-yl]biphenyl-3-yl}-1H-1,2,3-triazole;
4′-[2-methyl-4-(trifluoromethyl)-1H-imidazol-1-yl]-3′-(5-{4-[(trifluoromethyl)oxy]phenyl}-1H-1,2,3-triazol-1-yl)biphenyl-3-sulfonamide;
5-(1-{3′-(methylsulfonyl)-4-[2-methyl-4-(trifluoromethyl)-1H-imidazol-1-yl]biphenyl-3-yl}-1H-1,2,3-triazol-5-yl)-1H-indole;
5-{4-fluoro-3-[(trifluoromethyl)oxy]phenyl}-1-{3′-(methylsulfonyl)-4-[2-methyl-4-(trifluoromethyl)-1H-imidazol-1-yl]biphenyl-3-yl}-1H-1,2,3-triazole;
4-(1-{3′-(methylsulfonyl)-4-[2-methyl-4-(trifluoromethyl)-1H-imidazol-1-yl]biphenyl-3-yl}-1H-1,2,3-triazol-5-yl)benzonitrile;
{3-fluoro-3′-[5-(4-methylphenyl)-1H-1,2,3-triazol-1-yl]-5-(methylsulfonyl)-4′-[2-methyl-4-(trifluoromethyl)-1H-imidazol-1-yl]biphenyl-4-yl}methanol;
1-{3′-(methylsulfonyl)-4-[2-methyl-4-(trifluoromethyl)-1H-imidazol-1-yl]biphenyl-3-yl}-5-{4-[(trifluoromethyl)oxy]phenyl}-1H-1,2,3-triazole;
5-(4-methylphenyl)-1-{3′-(methylsulfonyl)-4-[2-methyl-4-(trifluoromethyl)-1H-imidazol-1-yl]biphenyl-3-yl}-1H-1,2,3-triazole;
1-{3′-(methylsulfonyl)-4-[2-methyl-4-(trifluoromethyl)-1H-imidazol-1-yl]biphenyl-3-yl}-5-(4-pyrrolidin-1-ylphenyl)-1H-1,2,3-triazole;
{3′-[5-(2,2-difluoro-1,3-benzodioxol-5-yl)-1H-1,2,3-triazol-1-yl]-3-fluoro-5-(methylsulfonyl)-4′-[2-methyl-4-(trifluoromethyl)-1H-imidazol-1-yl]biphenyl-4-yl}methanol;
5-{3-chloro-4-[(trifluoromethyl)oxy]phenyl}-1-{3′-(methylsulfonyl)-4-[2-methyl-4-(trifluoromethyl)-1H-imidazol-1-yl]biphenyl-3-yl}-1H-1,2,3-triazole;
5-{3-fluoro-4-[(trifluoromethyl)oxy]phenyl}-1-{3′-(methylsulfonyl)-4-[2-methyl-4-(trifluoromethyl)-1H-imidazol-1-yl]biphenyl-3-yl}-1H-1,2,3-triazole;
1-{4-[5-chloro-2-methyl-4-(trifluoromethyl)-1H-imidazol-1-yl]-3′-(methylsulfonyl)biphenyl-3-yl}-5-{4-[(trifluoromethyl)oxy]phenyl}-1H-1,2,3-triazole;
{3-fluoro-3′-(5-{3-fluoro-4-[(trifluoromethyl)oxy]phenyl}-1H-1,2,3-triazol-1-yl)-5-(methylsulfonyl)-4′-[2-methyl-4-(trifluoromethyl)-1H-imidazol-1-yl]biphenyl-4-yl}methanol;
1-{3′-(methylsulfonyl)-4-[2-methyl-4-(trifluoromethyl)-1H-imidazol-1-yl]biphenyl-3-yl}-5-{2-methyl-4-(trifluoromethyl)oxy]phenyl}-1H-1,2,3-triazole;
{3′-[5-(4-chlorophenyl)-1H-1,2,3-triazol-1-yl]-3-fluoro-5-(methylsulfonyl)-4′-[2-methyl-4-(trifluoromethyl)-1H-imidazol-1-yl]biphenyl-4-yl}methanol;
1-{4-[5-chloro-2-methyl-4-(trifluoromethyl)-1H-imidazol-1-yl]-3′-(methylsulfonyl)biphenyl-3-yl}-5-(2,4-dichlorophenyl)-1H-1,2,3-triazole;
{3-chloro-4′-[5-chloro-2-methyl-4-(trifluoromethyl)-1H-imidazol-1-yl]-5-(methylsulfonyl)-3′-(5-{4-[(trifluoromethyl)oxy]phenyl}-1H-1,2,3-triazol-1-yl)biphenyl-4-yl}methanol;
5-{2-fluoro-4-[(trifluoromethyl)oxy]phenyl}-1-{3′-(methylsulfonyl)-4-[2-methyl-4-(trifluoromethyl)-1H-imidazol-1-yl]biphenyl-3-yl}-1H-1,2,3-triazole;
1-{3′-(methylsulfonyl)-4-[2-methyl-4-(trifluoromethyl)-1H-imidazol-1-yl]biphenyl-3-yl}-5-[4-(trifluoromethyl)phenyl]-1H-1,2,3-triazole;
{3-chloro-3′-[5-(4-methylphenyl)-1H-1,2,3-triazol-1-yl]-5-(methylsulfonyl)-4′-[2-methyl-4-(trifluoromethyl)-1H-imidazol-1-yl]biphenyl-4-yl}methanol;
5-[4-chloro-3-(trifluoromethyl)phenyl]-1-{3′-(methylsulfonyl)-4-[2-methyl-4-(trifluoromethyl)-1H-imidazol-1-yl]biphenyl-3-yl}-1H-1,2,3-triazole;
5-{4-[(difluoromethyl)oxy]phenyl}-1-{3′-(methylsulfonyl)-4-[2-methyl-4-(trifluoromethyl)-1H-imidazol-1-yl]biphenyl-3-yl}-1H-1,2,3-triazole;
5-(4-chlorophenyl)-1-{3′-(methylsulfonyl)-4-[2-methyl-4-(trifluoromethyl)-1H-imidazol-1-yl]biphenyl-3-yl}-1H-1,2,3-triazole;
{3′-[5-(2,4-dichlorophenyl)-1H-1,2,3-triazol-1-yl]-3-fluoro-5-(methylsulfonyl)-4′-[2-methyl-4-(trifluoromethyl)-1H-imidazol-1-yl]biphenyl-4-yl}methanol;
5-{2-chloro-4-[(trifluoromethyl)oxy]phenyl}-1-{3′-(methylsulfonyl)-4-[2-methyl-4-(trifluoromethyl)-1H-imidazol-1-yl]biphenyl-3-yl}-1H-1,2,3-triazole;
5-(4-chloro-3-fluorophenyl)-1-{3′-(methylsulfonyl)-4-[2-methyl-4-(trifluoromethyl)-1H-imidazol-1-yl]biphenyl-3-yl}-1H-1,2,3-triazole;
{3-chloro-3′-[5-(4-chlorophenyl)-1H-1,2,3-triazol-1-yl]-5-(methylsulfonyl)-4′-[2-methyl-4-(trifluoromethyl)-1H-imidazol-1-yl]biphenyl-4-yl}methanol;
1-{3′-(methylsulfonyl)-4-[2-methyl-4-(trifluoromethyl)-1H-imidazol-1-yl]biphenyl-3-yl}-5-(3,4,5-trifluorophenyl)-1H-1,2,3-triazole;
5-[4-(1-methylethyl)phenyl]-1-{3′-(methylsulfonyl)-4-[2-methyl-4-(trifluoromethyl)-1H-imidazol-1-yl]biphenyl-3-yl}-1H-1,2,3-triazole;
5-(2,2-difluoro-1,3-benzodioxol-5-yl)-1-{3′-(methylsulfonyl)-4-[2-methyl-4-(trifluoromethyl)-1H-imidazol-1-yl]biphenyl-3-yl}-1H-1,2,3-triazole;
5-[4-(methyloxy)-3-(trifluoromethyl)phenyl]-1-{3′-(methylsulfonyl)-4-[2-methyl-4-(trifluoromethyl)-1H-imidazol-1-yl]biphenyl-3-yl}-1H-1,2,3-triazole;
5-(3,4-dichlorophenyl)-1-{3′-(methylsulfonyl)-4-[2-methyl-4-(trifluoromethyl)-1H-imidazol-1-yl]biphenyl-3-yl}-1H-1,2,3-triazole;
5-(2,4-dichlorophenyl)-1-{3′-(methylsulfonyl)-4-[2-methyl-4-(trifluoromethyl)-1H-imidazol-1-yl]biphenyl-3-yl}-1H-1,2,3-triazole;
5-fluoro-2-(1-{3′-(methylsulfonyl)-4-[2-methyl-4-(trifluoromethyl)-1H-imidazol-1-yl]biphenyl-3-yl}-1H-1,2,3-triazol-5-yl)pyridine;
{3′-(5-{3-chloro-4-(trifluoromethyl)oxy]phenyl}-1H-1,2,3-triazol-1-yl)-3-fluoro-5-(methylsulfonyl)-4′-[2-methyl-4-(trifluoromethyl)-1H-imidazol-1-yl]biphenyl-4-yl}methanol;
{3-chloro-3′-[5-(3,4-dichlorophenyl)-1H-1,2,3-triazol-1-yl]-5-(methylsulfonyl)-4′-[2-methyl-4-(trifluoromethyl)-1H-imidazol-1-yl]biphenyl-4-yl}methanol;
5-(1-{3′-(methylsulfonyl)-4-[2-methyl-4-(trifluoromethyl)-1H-imidazol-1-yl]biphenyl-3-yl}-1H-1,2,3-triazol-5-yl)-2-(trifluoromethyl)pyridine;
{3′-[5-(3,4-dichlorophenyl)-1H-1,2,3-triazol-1-yl]-3-fluoro-5-(methylsulfonyl)-4′-[2-methyl-4-(trifluoromethyl)-1H-imidazol-1-yl]biphenyl-4-yl}methanol;
methyl 4-(1-{3′-(methylsulfonyl)-4-[2-methyl-4-(trifluoromethyl)-1H-imidazol-1-yl]biphenyl-3-yl}-1H-1,2,3-triazol-5-yl)benzoate;
1-[4-(2,4-dimethyl-1H-imidazol-1-yl)-3′-(methylsulfonyl)biphenyl-3-yl]-5-{4-[(trifluoromethyl)oxy]phenyl}-1H-1,2,3-triazole;
[4′-(2,4-dimethyl-1H-imidazol-1-yl)-3-fluoro-5-(methylsulfonyl)-3′-(5-{4-[(trifluoromethyl)oxy]phenyl}-1H-1,2,3-triazol-1-yl)biphenyl-4-yl]methanol;
1-{4′-[(ethyloxy)methyl]-3′-(methylsulfonyl)-4-[2-methyl-4-(trifluoromethyl)-1H-imidazol-1-yl]biphenyl-3-yl}-5-{4-[(trifluoromethyl)oxy]phenyl}-1H-1,2,3-triazole;
5-(1-benzofuran-5-yl)-1-{3′-(methylsulfonyl)-4-[2-methyl-4-(trifluoromethyl)-1H-imidazol-1-yl]biphenyl-3-yl}-1H-1,2,3-triazole;
N-{[3-(methylsulfonyl)-4′-[2-methyl-4-(trifluoromethyl)-1H-imidazol-1-yl]-3′-(5-{4-[(trifluoromethyl)oxy]phenyl}-1H-1,2,3-triazol-1-yl)biphenyl-4-yl]methyl}methanesulfonamide;
1-{3′-fluoro-4′-[(methyloxy)methyl]-5′-(methylsulfonyl)-4-[2-methyl-4-(trifluoromethyl)-1H-imidazol-1-yl]biphenyl-3-yl}-5-{4-[(trifluoromethyl)oxy]phenyl}-1H-1,2,3-triazole;
1-{4′-[(methyloxy)methyl]-3′-(methylsulfonyl)-4-[2-methyl-4-(trifluoromethyl)-1H-imidazol-1-yl]biphenyl-3-yl}-5-{4-[(trifluoromethyl)oxy]phenyl}-1H-1,2,3-triazole;
[3-(methylsulfonyl)-4′-[2-methyl-4-(trifluoromethyl)-1H-imidazol-1-yl]-3′-(5-{4-[(trifluoromethyl)oxy]phenyl}-1H-1,2,3-triazol-1-yl)biphenyl-4-yl]methanol;
1-{3′-fluoro-5′-(methylsulfonyl)-4-[2-methyl-4-(trifluoromethyl)-1H-imidazol-1-yl]biphenyl-3-yl}-5-{4-[(trifluoromethyl)oxy]phenyl}-1H-1,2,3-triazole;
[3-(ethylsulfonyl)-5-fluoro-4′-[2-methyl-4-(trifluoromethyl)-1H-imidazol-1-yl]-3′-(5-{4-[(trifluoromethyl)oxy]phenyl}-1H-1,2,3-triazol-1-yl)biphenyl-4-yl]methanol;
5-[2,4-bis(methyloxy)phenyl]-1-{3′-(methylsulfonyl)-4-[2-methyl-4-(trifluoromethyl)-1H-imidazol-1-yl]biphenyl-3-yl}-1H-1,2,3-triazole;
1-{4′-(methyloxy)-3′-(methylsulfonyl)-4-[2-methyl-4-(trifluoromethyl)-1H-imidazol-1-yl]biphenyl-3-yl}-5-{4-[(trifluoromethyl)oxy]phenyl}-1H-1,2,3-triazole;
2-methyl-2-{4′-[2-methyl-4-(trifluoromethyl)-1H-imidazol-1-yl]-3′-(5-{4-[(trifluoromethyl)oxy]phenyl}-1H-1,2,3-triazol-1-yl)biphenyl-3-yl}propanoic acid
1-{3′-[(1-methylethyl)sulfonyl]-4-[2-methyl-4-(trifluoromethyl)-1H-imidazol-1-yl]biphenyl-3-yl}-5-{4-[(trifluoromethyl)oxy]phenyl}-1H-1,2,3-triazole;
1-{6-fluoro-3′-(methylsulfonyl)-4-[2-methyl-4-(trifluoromethyl)-1H-imidazol-1-yl]biphenyl-3-yl}-5-{4-(trifluoromethyl)oxy]phenyl}-1H-1,2,3-triazole;
5-(4-chlorophenyl)-1-{6-fluoro-3′-(methylsulfonyl)-4-[2-methyl-4-(trifluoromethyl)-1H-imidazol-1-yl]biphenyl-3-yl}-1H-1,2,3-triazole;
5-(2,4-dichlorophenyl)-1-{6-fluoro-3′-(methylsulfonyl)-4-[2-methyl-4-(trifluoromethyl)-1H-imidazol-1-yl]biphenyl-3-yl}-1H-1,2,3-triazole;
{2′-chloro-3′-[5-(4-chlorophenyl)-1H-1,2,3-triazol-1-yl]-3-fluoro-5-(methylsulfonyl)-4′-[2-methyl-4-(trifluoromethyl)-1H-imidazol-1-yl]biphenyl-4-yl}methanol;
{2′-chloro-3′-[5-(2,4-dichlorophenyl)-1H-1,2,3-triazol-1-yl]-3-fluoro-5-(methylsulfonyl)-4′-[2-methyl-4-(trifluoromethyl)-1H-imidazol-1-yl]biphenyl-4-yl}methanol;
N,N-dimethyl-3′-{5-[1-(4-methylphenyl)ethyl]-1H-1,2,3-triazol-1-yl}-4′-[2-methyl-4-(trifluoromethyl)-1H-imidazol-1-yl]biphenyl-3-sulfonamide;
(3′-{5-[1-(4-chlorophenyl)cyclopropyl]-1H-1,2,3-triazol-1-yl}-3-fluoro-5-(methylsulfonyl)-4′-[2-methyl-4-(trifluoromethyl)-1H-imidazol-1-yl]biphenyl-4-yl)methanol;
1-{3′-(ethylsulfonyl)-4-[2-methyl-4-(trifluoromethyl)-1H-imidazol-1-yl]biphenyl-3-yl}-5-[1-(4-methylphenyl)ethyl]-1H-1,2,3-triazole;
5-[1-(4-chlorophenyl)ethyl]-1-{3′-(methylsulfonyl)-4-[2-methyl-4-(trifluoromethyl)-1H-imidazol-1-yl]biphenyl-3-yl}-1H-1,2,3-triazole;
(3′-{5-[1-(4-chlorophenyl)ethyl]-1H-1,2,3-triazol-1-yl}-3-fluoro-5-(methylsulfonyl)-4′-[2-methyl-4-(trifluoromethyl)-1H-imidazol-1-yl]biphenyl-4-yl)methanol;
(3-chloro-3′-{5-[1-(4-methylphenyl)ethyl]-1H-1,2,3-triazol-1-yl}-5-(methylsulfonyl)-4′-[2-methyl-4-(trifluoromethyl)-1H-imidazol-1-yl]biphenyl-4-yl)methanol;
5-[1-(4-methylphenyl)ethyl]-1-{3′-methylsulfonyl)-4-[2-methyl-4-(trifluoromethyl)-1H-imidazol-1-yl]biphenyl-3-yl}-1H-1,2,3-triazole;
5-[1-(4-chlorophenyl)cyclopropyl]-1-{3′-(methylsulfonyl)-4-[2-methyl-4-(trifluoromethyl)-1H-imidazol-1-yl]biphenyl-3-yl}-1H-1,2,3-triazole;
(3′-{5-[(4-chlorophenyl)methyl]-1H-1,2,3-triazol-1-yl}-3-fluoro-5-(methylsulfonyl)-4′-[2-methyl-4-(trifluoromethyl)-1H-imidazol-1-yl]biphenyl-4-yl)methanol;
2-(3′-{5-[(3-chlorophenyl)methyl]-1H-1,2,3-triazol-1-yl}-4′-[2-methyl-4-(trifluoromethyl)-1H-imidazol-1-yl]biphenyl-3-yl)-2-methylpropanamide;
5-[(3,4-dichlorophenyl)methyl]-1-{3′-(methylsulfonyl)-4-[2-methyl-4-(trifluoromethyl)-1H-imidazol-1-yl]biphenyl-3-yl}-1H-1,2,3-triazole;
1-{3′-(methylsulfonyl)-4-[2-methyl-4-(trifluoromethyl)-1H-imidazol-1-yl]biphenyl-3-yl}-5-(13-[(trifluoromethyl)oxy]phenyl}methyl)-1H-1,2,3-triazole;
(3-chloro-3′-{5-[(3-chlorophenyl)methyl]-1H-1,2,3-triazol-1-yl}-5-(methylsulfonyl)-4′-[2-methyl-4-(trifluoromethyl)-1H-imidazol-1-yl]biphenyl-4-yl)methanol;
1-{3′-(methylsulfonyl)-4-[2-methyl-4-(trifluoromethyl)-1H-imidazol-1-yl]biphenyl-3-yl}-5-(14-[(trifluoromethyl)oxy]phenyl}methyl)-1H-1,2,3-triazole;
2-(3′-{5-[(4-chlorophenyl)methyl]-1H-1,2,3-triazol-1-yl}-4′-[2-methyl-4-(trifluoromethyl)-1H-imidazol-1-yl]biphenyl-3-yl)-2-methylpropanamide;
(3′-{5-[(3-chlorophenyl)methyl]-1H-1,2,3-triazol-1-yl}-3-fluoro-5-(methylsulfonyl)-4′-[2-methyl-4-(trifluoromethyl)-1H-imidazol-1-yl]biphenyl-4-yl)methanol;
5-[(3-chlorophenyl)methyl]-1-{3′-(methylsulfonyl)-4-[2-methyl-4-(trifluoromethyl)-1H-imidazol-1-yl]biphenyl-3-yl}-1H-1,2,3-triazole;
(3-chloro-3′-{5-[(4-chlorophenyl)methyl]-1H-1,2,3-triazol-1-yl}-5-(methylsulfonyl)-4′-[2-methyl-4-(trifluoromethyl)-1H-imidazol-1-yl]biphenyl-4-yl)methanol;
1-{3′-(methylsulfonyl)-4-[2-methyl-4-(trifluoromethyl)-1H-imidazol-1-yl]biphenyl-3-yl}-5-({2-[(trifluoromethyl)oxy]phenyl}methyl)-1H-1,2,3-triazole;
5-[(4-fluorophenyl)methyl]-1-{3′-(methylsulfonyl)-4-[2-methyl-4-(trifluoromethyl)-1H-imidazol-1-yl]biphenyl-3-yl}-1H-1,2,3-triazole;
5-[(4-chlorophenyl)methyl]-1-{3′-(methylsulfonyl)-4-[2-methyl-4-(trifluoromethyl)-1H-imidazol-1-yl]biphenyl-3-yl}-1H-1,2,3-triazole;
5-[(4-methylphenyl)methyl]-1-{3′-(methylsulfonyl)-4-[2-methyl-4-(trifluoromethyl)-1H-imidazol-1-yl]biphenyl-3-yl}-1H-1,2,3-triazole;
5-[(4-methylcyclohexyl)methyl]-1-{3′-(methylsulfonyl)-4-[2-methyl-4-(trifluoromethyl)-1H-imidazol-1-yl]biphenyl-3-yl}-1H-1,2,3-triazole;
5-[2-(4-chlorophenyl)ethyl]-1-{3′-(methylsulfonyl)-4-[2-methyl-4-(trifluoromethyl)-1H-imidazol-1-yl]biphenyl-3-yl}-1H-1,2,3-triazole;
3′-{5-[1-(4-chlorophenyl)ethyl]-1H-1,2,3-triazol-1-yl}-4′-[4-(difluoromethyl)-2-methyl-1H-imidazol-1-yl]-N-methylbiphenyl-3-sulfonamide;
5-[1-(4-chlorophenyl)ethyl]-1-{4-[4-(difluoromethyl)-2-methyl-1H-imidazol-1-yl]-3′-(ethylsulfonyl)biphenyl-3-yl}-1H-1,2,3-triazole;
3′-{5-[1-(4-chlorophenyl)ethyl]-1H-1,2,3-triazol-1-yl}-4′-[4-(difluoromethyl)-2-methyl-1H-imidazol-1-yl]-N,N-dimethylbiphenyl-3-sulfonamide;
5-[1-(4-chlorophenyl)ethyl]-1-{4-[4-(difluoromethyl)-2-methyl-1H-imidazol-1-yl]-3′-(methylsulfonyl)biphenyl-3-yl}-1H-1,2,3-triazole;
[3′-{5-[1-(4-chlorophenyl)ethyl]-1H-1,2,3-triazol-1-yl}-4′-[4-(difluoromethyl)-2-methyl-1H-imidazol-1-yl]-3-fluoro-5-(methylsulfonyl)biphenyl-4-yl]methanol;
2-methyl-2-{4′-[2-methyl-4-(trifluoromethyl)-1H-imidazol-1-yl]-3′-(5-{[4-(trifluoromethyl)phenyl]carbonyl}-1H-1,2,3-triazol-1-yl)biphenyl-3-yl}propanamide;
(1-{3′-fluoro-4′-(hydroxymethyl)-5′-(methylsulfonyl)-4-[2-methyl-4-(trifluoromethyl)-1H-imidazol-1-yl]biphenyl-3-yl}-1H-1,2,3-triazol-5-yl)[3-(trifluoromethyl)phenyl]methanone;
(1-{3′-(methylsulfonyl)-4-[2-methyl-4-(trifluoromethyl)-1H-imidazol-1-yl]biphenyl-3-yl}-1H-1,2,3-triazol-5-yl)[3-(trifluoromethyl)phenyl] methanone;
(1-{3′-(methylsulfonyl)-4-[2-methyl-4-(trifluoromethyl)-1H-imidazol-1-yl]biphenyl-3-yl}-1H-1,2,3-triazol-5-yl)[4-(trifluoromethyl)phenyl]methanone;
2-(3′-{5-[(4-chlorophenyl)carbonyl]-1H-1,2,3-triazol-1-yl}-4′-[2-methyl-4-(trifluoromethyl)-1H-imidazol-1-yl]biphenyl-3-yl)-2-methylpropanamide;
(4-chlorophenyl)(1-{3′-fluoro-4′-(hydroxymethyl)-5′-(methylsulfonyl)-4-[2-methyl-4-(trifluoromethyl)-1H-imidazol-1-yl]biphenyl-3-yl}-1H-1,2,3-triazol-5-yl)methanone;
(1-{3′-fluoro-4′-(hydroxymethyl)-5′-(methylsulfonyl)-4-[2-methyl-4-(trifluoromethyl)-1H-imidazol-1-yl]biphenyl-3-yl}-1H-1,2,3-triazol-5-yl)[4-(trifluoromethyl)phenyl]methanone;
5-[(4-chlorophenyl)(difluoro)methyl]-1-{3′-(ethylsulfonyl)-4-[2-methyl-4-(trifluoromethyl)-1H-imidazol-1-yl]biphenyl-3-yl}-1H-1,2,3-triazole;
1-{4-[2-chloro-4-(trifluoromethyl)-1H-imidazol-1-yl]-3′-(methylsulfonyl)biphenyl-3-yl}-5-(2,4-dichlorophenyl)-1H-1,2,3-triazole;
{3′-[5-(4-chlorophenyl)-1H-1,2,3-triazol-1-yl]-4′-[2-chloro-4-(trifluoromethyl)-1H-imidazol-1-yl]-3-fluoro-5-(methylsulfonyl)biphenyl-4-yl}methanol;
{4′-[2-chloro-4-(trifluoromethyl)-1H-imidazol-1-yl]-3′-[5-(2,4-dichlorophenyl)-1H-1,2,3-triazol-1-yl]-3-fluoro-5-methylsulfonyl)biphenyl-4-yl}methanol;
1-{4-[2-chloro-4-(trifluoromethyl)-1H-imidazol-1-yl]-3′-(methylsulfonyl)biphenyl-3-yl}-5-{4-[(trifluoromethyl)oxy]phenyl}-1H-1,2,3-triazole;
[4′-(4-chloro-2-methyl-1H-imidazol-1-yl)-3-fluoro-5-(methylsulfonyl)-3′-(5-{4-[(trifluoromethyl)oxy]phenyl}-1H-1,2,3-triazol-1-yl)biphenyl-4-yl]methanol;
[4′-(4-chloro-2-methyl-1H-imidazol-1-yl)-3′-[5-(4-chlorophenyl)-1H-1,2,3-triazol-1-yl]-3-fluoro-5-(methylsulfonyl)biphenyl-4-yl]methanol;
1-[4-(4-chloro-2-methyl-1H-imidazol-1-yl)-3′-(methylsulfonyl)biphenyl-3-yl]-5-{4-[(trifluoromethyl)oxy]phenyl}-1H-1,2,3-triazole;
1-[4-(4-chloro-2-methyl-1H-imidazol-1-yl)-3′-(methylsulfonyl)biphenyl-3-yl]-5-(4-chlorophenyl)-1H-1,2,3-triazole;
1-[4-(4,5-dichloro-2-methyl-1H-imidazol-1-yl)-3′-(methylsulfonyl)biphenyl-3-yl]-5-{4-[(trifluoromethyl)oxy]phenyl}-1H-1,2,3-triazole;
N-(1,1-dimethylethyl)-2-methyl-1-[3′-methylsulfonyl)-3-(5-{4-[(trifluoromethyl)oxy]phenyl}-1H-1,2,3-triazol-1-yl)biphenyl-4-yl]-1H-imidazole-4-carboxamide;
4′-[4-(difluoromethyl)-2-methyl-1H-imidazol-1-yl]-N-methyl-3′-(5-{4-[(trifluoromethyl)oxy]phenyl}-1H-1,2,3-triazol-1-yl)biphenyl-3-sulfonamide;
4′-[4-(difluoromethyl)-2-methyl-1H-imidazol-1-yl]-N,N-dimethyl-3′-(5-{4-[(trifluoromethyl)oxy]phenyl}-1H-1,2,3-triazol-1-yl)biphenyl-3-sulfonamide;
2-{4′-[4-(difluoromethyl)-2-methyl-1H-imidazol-1-yl]-3′-(5-{4-[(trifluoromethyl)oxy]phenyl}-1H-1,2,3-triazol-1-yl)biphenyl-3-yl}-2-methylpropanamide;
1-{4-[4-(difluoromethyl)-2-methyl-1H-imidazol-1-yl]-3′-(ethylsulfonyl)biphenyl-3-yl}-5-{4-[(trifluoromethyl)oxy]phenyl}-1H-1,2,3-triazole;
{3-chloro-4′-[4-(difluoromethyl)-2-methyl-1H-imidazol-1-yl]-5-(methylsulfonyl)-3′-(5-{4-[(trifluoromethyl)oxy]phenyl}-1H-1,2,3-triazol-1-yl)biphenyl-4-yl}methanol;
{4′-[4-(difluoromethyl)-2-methyl-1H-imidazol-1-yl]-3-(methylsulfonyl)-3′-(5-{4-[(trifluoromethyl)oxy]phenyl}-1H-1,2,3-triazol-1-yl)biphenyl-4-yl}methanol;
5-(4-chlorophenyl)-1-{4-[4-(difluoromethyl)-2-methyl-1H-imidazol-1-yl]-3′-(methylsulfonyl)biphenyl-3-yl}-1H-1,2,3-triazole;
5-(2,4-dichlorophenyl)-1-{4-[4-(difluoromethyl)-2-methyl-1H-imidazol-1-yl]-3′-methylsulfonyl)biphenyl-3-yl}-1H-1,2,3-triazole;
1-{4-[4-(1,1-difluoro-2-methylpropyl)-2-methyl-1H-imidazol-1-yl]-3′-(methylsulfonyl)biphenyl-3-yl}-5-{4-[(trifluoromethyl)oxy]phenyl}-1H-1,2,3-triazole;
1-{4-[4-(1,1-difluoroethyl)-2-methyl-1H-imidazol-1-yl]-3′-(methylsulfonyl)biphenyl-3-yl}-5-{4-[(trifluoromethyl)oxy]phenyl}-1H-1,2,3-triazole;
1-{4-[4-(difluoromethyl)-2-methyl-1H-imidazol-1-yl]-3′-(methylsulfonyl)biphenyl-3-yl}-5-[4-(methyloxy)phenyl]-1H-1,2,3-triazole;
5-(2,2-difluoro-1,3-benzodioxol-5-yl)-1-{4-[4-(difluoromethyl)-2-methyl-1H-imidazol-1-yl]-3′-(methylsulfonyl)biphenyl-3-yl}-1H-1,2,3-triazole;
1-{4-[4-(difluoromethyl)-2-methyl-1H-imidazol-1-yl]-3′-(methylsulfonyl)biphenyl-3-yl}-5-(4-methylphenyl)-1H-1,2,3-triazole;
1-{4-[4-(difluoromethyl)-2-methyl-1H-imidazol-1-yl]-3′-(methylsulfonyl)biphenyl-3-yl}-5-{3-fluoro-4-[(trifluoromethyl)oxy]phenyl}-1H-1,2,3-triazole;
1-{4-[4-(difluoromethyl)-1H-imidazol-1-yl]-3′-(methylsulfonyl)biphenyl-3-yl}-5-{4-[(trifluoromethyl)oxy]phenyl}-1H-1,2,3-triazole;
1-{4-[4-(difluoromethyl)-2-methyl-1H-imidazol-1-yl]-3′-(methylsulfonyl)biphenyl-3-yl}-5-phenyl-1H-1,2,3-triazole;
1-{4-[5-chloro-4-(difluoromethyl)-2-methyl-1H-imidazol-1-yl]-3′-(methylsulfonyl)biphenyl-3-yl}-5-{4-[(trifluoromethyl)oxy]phenyl}-1H-1,2,3-triazole;
{4′-[5-chloro-4-(difluoromethyl)-2-methyl-1H-imidazol-1-yl]-3-fluoro-5-(methylsulfonyl)-3′-(5-{4-[(trifluoromethyl)oxy]phenyl}-1H-1,2,3-triazol-1-yl)biphenyl-4-yl}methanol;
{4′-[4-(1,1-difluoroethyl)-2-methyl-1H-imidazol-1-yl]-3-fluoro-5-(methylsulfonyl)-3′-(5-{4-[(trifluoromethyl)oxy]phenyl}-1H-1,2,3-triazol-1-yl)biphenyl-4-yl}methanol;
4′-[4-(1,1-difluoroethyl)-2-methyl-1H-imidazol-1-yl]-N-methyl-3′-(5-{4-[(trifluoromethyl)oxy]phenyl}-1H-1,2,3-triazol-1-yl)biphenyl-3-sulfonamide;
5-(4-chlorophenyl)-1-{4-[4-(1,1-difluoroethyl)-2-methyl-1H-imidazol-1-yl]-3′-(methylsulfonyl)biphenyl-3-yl}-1H-1,2,3-triazole;
1-{4-[4-(1,1-difluoroethyl)-2-methyl-1H-imidazol-1-yl]-3′-(ethylsulfonyl)biphenyl-3-yl}-5-{4-[(trifluoromethyl)oxy]phenyl}-1H-1,2,3-triazole;
{4′-[4-(1,1-difluoroethyl)-2-methyl-1H-imidazol-1-yl]-3-(methylsulfonyl)-3′-(5-{4-[(trifluoromethyl)oxy]phenyl}-1H-1,2,3-triazol-1-yl)biphenyl-4-yl}methanol;
1-{4-[4-(1,1-difluoroethyl)-2-methyl-1H-imidazol-1-yl]-3′-fluoro-4′-[(methyloxy)methyl]-5′-(methylsulfonyl)biphenyl-3-yl}-5-{4-[(trifluoromethyl)oxy]phenyl}-1H-1,2,3-triazole;
5-(2,4-dichlorophenyl)-1-{4-[4-(1,1-difluoroethyl)-2-methyl-1H-imidazol-1-yl]-3′-(methylsulfonyl)biphenyl-3-yl}-1H-1,2,3-triazole;
4′-[4-(1,1-difluoroethyl)-2-methyl-1H-imidazol-1-yl]-N,N-dimethyl-3′-(5-{4-[(trifluoromethyl)oxy]phenyl}-1H-1,2,3-triazol-1-yl)biphenyl-3-sulfonamide;
4′-[4-(1,1-difluoropropyl)-2-methyl-1H-imidazol-1-yl]-N,N-dimethyl-3′-(5-{4-[(trifluoromethyl)oxy]phenyl}-1H-1,2,3-triazol-1-yl)biphenyl-3-sulfonamide;
4′-[4-(1,1-difluoropropyl)-2-methyl-1H-imidazol-1-yl]-3′-(5-{4-[(trifluoromethyl)oxy]phenyl}-1H-1,2,3-triazol-1-yl)biphenyl-3-sulfonamide;
1-{4-[4-(1,1-difluoropropyl)-2-methyl-1H-imidazol-1-yl]-3′-(methylsulfonyl)biphenyl-3-yl}-5-{4-[(trifluoromethyl)oxy]phenyl}-1H-1,2,3-triazole;
4′-[4-(1,1-difluoropropyl)-2-methyl-1H-imidazol-1-yl]-N-methyl-3′-(5-{4-[(trifluoromethyl)oxy]phenyl}-1H-1,2,3-triazol-1-yl)biphenyl-3-sulfonamide;
{4′-[4-(1,1-difluoropropyl)-2-methyl-1H-imidazol-1-yl]-3-fluoro-5-(methylsulfonyl)-3′-(5-{4-[(trifluoromethyl)oxy]phenyl}-1H-1,2,3-triazol-1-yl)biphenyl-4-yl}methanol;
5-(4-chlorophenyl)-1-{5-fluoro-3′-(methylsulfonyl)-4-[4-(trifluoromethyl)-1H-imidazol-1-yl]biphenyl-3-yl}-1H-1,2,3-triazole;
1-{5-fluoro-3′-(methylsulfonyl)-4-[4-(trifluoromethyl)-1H-imidazol-1-yl]biphenyl-3-yl}-5-{4-[(trifluoromethyl)oxy]phenyl}-1H-1,2,3-triazole;
[3,3′-difluoro-5-(methylsulfonyl)-4′-[4-(trifluoromethyl)-1H-imidazol-1-yl]-5′-(5-{4-[(trifluoromethyl)oxy]phenyl}-1H-1,2,3-triazol-1-yl)biphenyl-4-yl]methanol;
{3′-[5-(2,4-dichlorophenyl)-1H-1,2,3-triazol-1-yl]-3,5′-difluoro-5-(methylsulfonyl)-4′-[4-(trifluoromethyl)-1H-imidazol-1-yl]biphenyl-4-yl}methanol;
5-(2,4-dichlorophenyl)-1-{5-fluoro-3′-(methylsulfonyl)-4-[4-(trifluoromethyl)-1H-imidazol-1-yl]biphenyl-3-yl}-1H-1,2,3-triazole;
1-{5-chloro-3′-(methylsulfonyl)-4-[4-(trifluoromethyl)-1H-imidazol-1-yl]biphenyl-3-yl}-5-{4-[(trifluoromethyl)oxy]phenyl}-1H-1,2,3-triazole;
[3′-chloro-3-fluoro-5-(methylsulfonyl)-4′-[4-(trifluoromethyl)-1H-imidazol-1-yl]-5′-(5-{4-[(trifluoromethyl)oxy]phenyl}-1H-1,2,3-triazol-1-yl)biphenyl-4-yl]methanol;
1-{5-chloro-3′-(methylsulfonyl)-4-[2-methyl-4-(trifluoromethyl)-1H-imidazol-1-yl]biphenyl-3-yl}-5-{4-[(trifluoromethyl)oxy]phenyl}-1H-1,2,3-triazole;
[3′-chloro-3-fluoro-5-(methylsulfonyl)-4′-[2-methyl-4-(trifluoromethyl)-1H-imidazol-1-yl]-5′-(5-{4-[(trifluoromethyl)oxy]phenyl}-1H-1,2,3-triazol-1-yl)biphenyl-4-yl]methanol;
[3-chloro-3′-fluoro-5-(methylsulfonyl)-4′-[2-methyl-4-(trifluoromethyl)-1H-imidazol-1-yl]-5′-(5-{4-[(trifluoromethyl)oxy]phenyl}-1H-1,2,3-triazol-1-yl)biphenyl-4-yl]methanol;
{3′-[5-(4-chlorophenyl)-1H-1,2,3-triazol-1-yl]-3,5′-difluoro-5-(methylsulfonyl)-4′-[2-methyl-4-(trifluoromethyl)-1H-imidazol-1-yl]biphenyl-4-yl}methanol;
1-{5-fluoro-4-[2-methyl-4-(trifluoromethyl)-1H-imidazol-1-yl]-3′-(pyrrolidin-1-ylsulfonyl)biphenyl-3-yl}-5-{4-[(trifluoromethyl)oxy]phenyl}-1H-1,2,3-triazole;
{3′-[5-(2,4-dichlorophenyl)-1H-1,2,3-triazol-1-yl]-3,5′-difluoro-5-(methylsulfonyl)-4′-[2-methyl-4-(trifluoromethyl)-1H-imidazol-1-yl]biphenyl-4-yl}methanol;
1-{5-fluoro-3′-(methylsulfonyl)-4-[2-methyl-4-(trifluoromethyl)-1H-imidazol-1-yl]biphenyl-3-yl}-5-{4-[(trifluoromethyl)oxy]phenyl}-1H-1,2,3-triazole;
[3,3′-difluoro-5-(methylsulfonyl)-4′-[2-methyl-4-(trifluoromethyl)-1H-imidazol-1-yl]-5′-(5-{4-[(trifluoromethyl)oxy]phenyl}-1H-1,2,3-triazol-1-yl)biphenyl-4-yl]methanol;
[3′-fluoro-3-(methylsulfonyl)-4′-[2-methyl-4-(trifluoromethyl)-1H-imidazol-1-yl]-5′-(5-{4-[(trifluoromethyl)oxy]phenyl}-1H-1,2,3-triazol-1-yl)biphenyl-4-yl]methanol;
5-(4-chlorophenyl)-1-{5-fluoro-3′-(methylsulfonyl)-4-[2-methyl-4-(trifluoromethyl)-1H-imidazol-1-yl]biphenyl-3-yl}-1H-1,2,3-triazole;
3′-fluoro-N-methyl-4′-[2-methyl-4-(trifluoromethyl)-1H-imidazol-1-yl]-5′-(5-{4-[(trifluoromethyl)oxy]phenyl}-1H-1,2,3-triazol-1-yl)biphenyl-3-sulfonamide;
1-{3′,5-difluoro-4′-[(methyloxy)methyl]-5′-(methylsulfonyl)-4-[2-methyl-4-(trifluoromethyl)-1H-imidazol-1-yl]biphenyl-3-yl}-5-{4-[(trifluoromethyl)oxy]phenyl}-1H-1,2,3-triazole;
5-(4-chlorophenyl)-1-{3′,5-difluoro-4′-[(methyloxy)methyl]-5′-(methylsulfonyl)-4-[2-methyl-4-(trifluoromethyl)-1H-imidazol-1-yl]biphenyl-3-yl}-1H-1,2,3-triazole;
3′-fluoro-N-(2-hydroxyethyl)-4′-[2-methyl-4-(trifluoromethyl)-1H-imidazol-1-yl]-5′-(5-{4-[(trifluoromethyl)oxy]phenyl}-1H-1,2,3-triazol-1-yl)biphenyl-3-sulfonamide;
3′-[5-(4-chlorophenyl)-1H-1,2,3-triazol-1-yl]-5′-fluoro-N-methyl-4′-[2-methyl-4-(trifluoromethyl)-1H-imidazol-1-yl]biphenyl-3-sulfonamide;
{3′-[5-(4-chlorophenyl)-1H-1,2,3-triazol-1-yl]-5′-fluoro-3-(methylsulfonyl)-4′-[2-methyl-4-(trifluoromethyl)-1H-imidazol-1-yl]biphenyl-4-yl}methanol;
(3-fluoro-4′-(2-methyl-4-(trifluoromethyl)-1H-imidazol-1-yl)-5-(methylsulfonyl)-3′-(5-(4-(trifluoromethoxy)phenyl)-1H-1,2,3-triazol-1-yl)-[1,1′-biphenyl]-4-yl)methanol;
(3-chloro-4′-(2-methyl-4-(trifluoromethyl)-1H-imidazol-1-yl)-5-(methylsulfonyl)-3′-(5-(4-(trifluoromethoxy)phenyl)-1H-1,2,3-triazol-1-yl)-[1,1′-biphenyl]-4-yl)methanol;
N-methyl-4′-(2-methyl-4-(trifluoromethyl)-1H-imidazol-1-yl)-3′-(5-(4-(trifluoromethoxy)phenyl)-1H-1,2,3-triazol-1-yl)-[1,1′-biphenyl]-3-sulfonamide;
N,N-dimethyl-4′-(2-methyl-4-(trifluoromethyl)-1H-imidazol-1-yl)-3′-(5-(4-(trifluoromethoxy)phenyl)-1H-1,2,3-triazol-1-yl)-[1,1′-biphenyl]-3-sulfonamide;
1-(3′-(ethylsulfonyl)-4-(2-methyl-4-(trifluoromethyl)-1H-imidazol-1-yl)-[1,1′-biphenyl]-3-yl)-5-(4-(trifluoromethoxy)phenyl)-1H-1,2,3-triazole;
(3′-(5-(benzofuran-5-yl)-1H-1,2,3-triazol-1-yl)-3-fluoro-4′-(2-methyl-4-(trifluoromethyl)-1H-imidazol-1-yl)-5-(methylsulfonyl)-[1,1′-biphenyl]-4-yl)methanol;
5-(2-chlorobenzyl)-1-(4-(2-methyl-4-(trifluoromethyl)-1H-imidazol-1-yl)-3′-(methylsulfonyl)-[1,1′-biphenyl]-3-yl)-1H-1,2,3-triazole;
(4′-(4-(difluoromethyl)-2-methyl-1H-imidazol-1-yl)-3-fluoro-5-(methylsulfonyl)-3′-(5-(4-(trifluoromethoxy)phenyl)-1H-1,2,3-triazol-1-yl)-[1,1′-biphenyl]-4-yl)methanol;
(4′-(4-(1,1-difluoro-2-methylpropyl)-2-methyl-1H-imidazol-1-yl)-3-fluoro-5-(methylsulfonyl)-3′-(5-(4-(trifluoromethoxy)phenyl)-1H-1,2,3-triazol-1-yl)-[1,1′-biphenyl]-4-yl)methanol;
5-(2-(4-chlorophenyl)propan-2-yl)-1-(4-(2-methyl-4-(trifluoromethyl)-1H-imidazol-1-yl)-3′-(methylsulfonyl)-[1,1′-biphenyl]-3-yl)-1H-1,2,3-triazole;
(3′-(5-(2-(4-chlorophenyl)propan-2-yl)-1H-1,2,3-triazol-1-yl)-3-fluoro-4′-(2-methyl-4-(trifluoromethyl)-1H-imidazol-1-yl)-5-(methylsulfonyl)-[1,1′-biphenyl]-4-yl)methanol;
5-(1-(4-chlorophenyl)-1-fluoroethyl)-1-(4-(2-methyl-4-(trifluoromethyl)-1H-imidazol-1-yl)-3′-(methylsulfonyl)-[1,1′-biphenyl]-3-yl)-1H-1,2,3-triazole;
5-(4-chlorophenyl)-1-(4-(2-(difluoromethyl)-4-(trifluoromethyl)-1H-imidazol-1-yl)-3′-(methylsulfonyl)-[1,1′-biphenyl]-3-yl)-1H-1,2,3-triazole;
1-(4-(2-(difluoromethyl)-4-(trifluoromethyl)-1H-imidazol-1-yl)-3′-(methylsulfonyl)-[1,1′-biphenyl]-3-yl)-5-(4-(trifluoromethoxy)phenyl)-1H-1,2,3-triazole;
1-(4-(2-ethyl-4-(trifluoromethyl)-1H-imidazol-1-yl)-3′-(methylsulfonyl)-[1,1′-biphenyl]-3-yl)-5-(4-(trifluoromethoxy)phenyl)-1H-1,2,3-triazole;
5-(4-chlorophenyl)-1-(4-(4-cyclopropyl-2-methyl-1H-imidazol-1-yl)-3′-(methylsulfonyl)-[1,1′-biphenyl]-3-yl)-1H-1,2,3-triazole;
1-(4-(4-cyclopropyl-2-methyl-1H-imidazol-1-yl)-3′-(methylsulfonyl)-[1,1′-biphenyl]-3-yl)-5-(4-(trifluoromethoxy)phenyl)-1H-1,2,3-triazole;
5-(4-chlorophenyl)-1-(4-(4-(difluoromethyl)-2,5-dimethyl-1H-imidazol-1-yl)-3′-(methylsulfonyl)-[1,1′-biphenyl]-3-yl)-1H-1,2,3-triazole;
(3′-(5-(4-chlorophenyl)-1H-1,2,3-triazol-1-yl)-4′-(4-(difluoromethyl)-2,5-dimethyl-1H-imidazol-1-yl)-3-fluoro-5-(methylsulfonyl)-[1,1′-biphenyl]-4-yl)methanol;
1-(4-(4-(difluoromethyl)-2,5-dimethyl-1H-imidazol-1-yl)-3′-(methylsulfonyl)-[1,1′-biphenyl]-3-yl)-5-(4-(trifluoromethoxy)phenyl)-1H-1,2,3-triazole;
(4′-(4-(difluoromethyl)-2,5-dimethyl-1H-imidazol-1-yl)-3-fluoro-5-methylsulfonyl)-3′-(5-(4-(trifluoromethoxy)phenyl)-1H-1,2,3-triazol-1-yl)-[1,1′-biphenyl]-4-yl)methanol;
2-methyl-1-(2-methyl-1-(3′-(methylsulfonyl)-3-(5-(4-(trifluoromethoxy)phenyl)-1H-1,2,3-triazol-1-yl)-[1,1′-biphenyl]-4-yl)-1H-imidazol-4-yl)propan-1-one;
1-(1-(3′-fluoro-4′-(hydroxymethyl)-5′-(methylsulfonyl)-3-(5-(4-(trifluoromethoxy)phenyl)-1H-1,2,3-triazol-1-yl)-[1,1′-biphenyl]-4-yl)-2-methyl-1H-imidazol-4-yl)-2-methylpropan-1-one;
2-(4′-(2-methyl-4-(trifluoromethyl)-1H-imidazol-1-yl)-3-(methylsulfonyl)-3′-(5-(4-(trifluoromethoxy)phenyl)-1H-1,2,3-triazol-1-yl)-[1,1′-biphenyl]-4-yl)ethanol;
2-(3-fluoro-4′-(2-methyl-4-(trifluoromethyl)-1H-imidazol-1-yl)-5-(methylsulfonyl)-3′-(5-(4-(trifluoromethoxy)phenyl)-1H-1,2,3-triazol-1-yl)-[1,1′-biphenyl]-4-yl)ethanol;
1-(3′-((methoxymethyl)sulfonyl)-4-(2-methyl-4-(trifluoromethyl)-1H-imidazol-1-yl)-[1,1′-biphenyl]-3-yl)-5-(4-(trifluoromethoxy)phenyl)-1H-1,2,3-triazole;
2-methyl-2-{4′-[2-methyl-4-(trifluoromethyl)-1H-imidazol-1-yl]-3′-(1-{4-[(trifluoromethyl)oxy]phenyl}-1H-1,2,3-triazol-5-yl)biphenyl-3-yl}propanamide;
5-{3′-(methylsulfonyl)-4-[2-methyl-4-(trifluoromethyl)-1H-imidazol-1-yl]biphenyl-3-yl}-1-{4-[(trifluoromethyl)oxy]phenyl}-1H-1,2,3-triazole;
5-{4-[5-chloro-2-methyl-4-(trifluoromethyl)-1H-imidazol-1-yl]-3′-(methylsulfonyl)biphenyl-3-yl}-1-(4-chlorophenyl)-1H-1,2,3-triazole;
[3-fluoro-5-(methylsulfonyl)-4′-[2-methyl-4-(trifluoromethyl)-1H-imidazol-1-yl]-3′-(1-{4-[(trifluoromethyl)oxy]phenyl}-1H-1,2,3-triazol-5-yl)biphenyl-4-yl]methanol;
1-(4-chlorobenzyl)-5-(4-(4-(difluoromethyl)-2-methyl-1H-imidazol-1-yl)-3′-(methylsulfonyl)-[1,1′-biphenyl]-3-yl)-1H-1,2,3-triazole;
3′-(1-(4-chlorobenzyl)-1H-1,2,3-triazol-5-yl)-4′-(4-(difluoromethyl)-2-methyl-1H-imidazol-1-yl)-N-methyl-[1,1′-biphenyl]-3-sulfonamide;
1-(4-chlorobenzyl)-5-(4-(4-(difluoromethyl)-2-methyl-1H-imidazol-1-yl)-3′-(ethylsulfonyl)-[1,1′-biphenyl]-3-yl)-1H-1,2,3-triazole;
(3′-(1-(4-chlorobenzyl)-1H-1,2,3-triazol-5-yl)-4′-(4-(difluoromethyl)-2-methyl-1H-imidazol-1-yl)-3-fluoro-5-(methylsulfonyl)-[1,1′-biphenyl]-4-yl)methanol;
1-(4-chlorophenyl)-5-{3′-(methylsulfonyl)-4-[2-methyl-4-(trifluoromethyl)-1H-imidazol-1-yl]biphenyl-3-yl}-1H-pyrazole;
(3′-(1-(4-(difluoromethoxy)phenyl)-3-methyl-1H-pyrazol-5-yl)-3-fluoro-4′-(2-methyl-4-(trifluoromethyl)-1H-imidazol-1-yl)-5-(methylsulfonyl)biphenyl-4-yl)methanol;
{3′-[1-(4-chlorophenyl)-1H-pyrazol-5-yl]-3-fluoro-5-(methylsulfonyl)-4′-[2-methyl-4-(trifluoromethyl)-1H-imidazol-1-yl]biphenyl-4-yl}methanol;
5-{3′-(methylsulfonyl)-4-[2-methyl-4-(trifluoromethyl)-1H-imidazol-1-yl]biphenyl-3-yl}-1-{4-[(trifluoromethyl)oxy]phenyl}-1H-pyrazole;
{3′-[1-(4-chlorophenyl)-3-methyl-1H-pyrazol-5-yl}-3-fluoro-5-(methylsulfonyl)-4′-[2-methyl-4-(trifluoromethyl)-1H-imidazol-1-yl]biphenyl-4-yl}methanol;
3-methyl-5-{3′-(methylsulfonyl)-4-[2-methyl-4-(trifluoromethyl)-1H-imidazol-1-yl]biphenyl-3-yl}-1-{4-[(trifluoromethyl)oxy]phenyl}-1H-pyrazole;
[3-fluoro-5-(methylsulfonyl)-4′-[2-methyl-4-(trifluoromethyl)-1H-imidazol-1-yl]-3′-(1-{4-[(trifluoromethyl)oxy]phenyl}-1H-pyrazol-5-yl)biphenyl-4-yl]methanol;
1-(2,2-difluorobenzo[d][1,3]dioxol-5-yl)-5-(4-(2-methyl-4-(trifluoromethyl)-1H-imidazol-1-yl)-3′-(methylsulfonyl)biphenyl-3-yl)-1H-pyrazole;
(3′-(1-(2,2-difluorobenzo[d][1,3]dioxol-5-yl)-1H-pyrazol-5-yl)-3-fluoro-4′-(2-methyl-4-(trifluoromethyl)-1H-imidazol-1-yl)-5-(methylsulfonyl)biphenyl-4-yl)methanol;
(3′-(1-(2,2-difluorobenzo[d][1,3]dioxol-5-yl)-1H-pyrazol-5-yl)-4′-(2-methyl-4-(trifluoromethyl)-1H-imidazol-1-yl)-3-(methylsulfonyl)biphenyl-4-yl)methanol;
2-chloro-5-(5-(4-(2-methyl-4-(trifluoromethyl)-1H-imidazol-1-yl)-3′-(methylsulfonyl)biphenyl-3-yl)-1H-pyrazol-1-yl)pyridine;
(3′-(1-(6-chloropyridin-3-yl)-1H-pyrazol-5-yl)-3-fluoro-4′-(2-methyl-4-(trifluoromethyl)-1H-imidazol-1-yl)-5-(methylsulfonyl)biphenyl-4-yl)methanol;
(3′-(1-(6-chloropyridin-3-yl)-1H-pyrazol-5-yl)-4′-(2-methyl-4-(trifluoromethyl)-1H-imidazol-1-yl)-3-(methylsulfonyl)biphenyl-4-yl)methanol;
(3′-(1-(2,2-difluorobenzo[d][1,3]dioxol-5-yl)-3-methyl-1H-pyrazol-5-yl)-3-fluoro-4′-(2-methyl-4-(trifluoromethyl)-1H-imidazol-1-yl)-5-(methylsulfonyl)biphenyl-4-yl)methanol;
(3′-(1-(2,2-difluorobenzo[d][1,3]dioxol-5-yl)-3-methyl-1H-pyrazol-5-yl)-4′-(2-methyl-4-(trifluoromethyl)-1H-imidazol-1-yl)-3-(methylsulfonyl)biphenyl-4-yl)methanol;
1-(4-(difluoromethoxy)phenyl)-3-methyl-5-(4-(2-methyl-4-(trifluoromethyl)-1H-imidazol-1-yl)-3′-(methylsulfonyl)biphenyl-3-yl)-1H-pyrazole;
(3′-(1-(4-(difluoromethoxy)phenyl)-3-methyl-1H-pyrazol-5-yl)-4′-(2-methyl-4-(trifluoromethyl)-1H-imidazol-1-yl)-3-(methylsulfonyl)-[1,1′-biphenyl]-4-yl)methanol;
4-(fluoromethyl)-3′-(3-methyl-1-(4-(trifluoromethoxy)phenyl)-1H-pyrazol-5-yl)-4′-(2-methyl-4-(trifluoromethyl)-1H-imidazol-1-yl)-5-(methylsulfonyl)-[1,1′-biphenyl]-3-ol;
1-(2,2-difluorobenzo[d][1,3]dioxol-5-yl)-3-methyl-5-(4-(2-methyl-4-(trifluoromethyl)-1H-imidazol-1-yl)-3′-(methylsulfonyl)-[1,1′-biphenyl]-3-yl)-1H-pyrazole;
5-(4-chlorophenyl)-4-{3′-(methylsulfonyl)-4-[2-methyl-4-(trifluoromethyl)-1H-imidazol-1-yl]biphenyl-3-yl}-1,3-oxazole;
{3′-[4-(4-chlorophenyl)-1,3-oxazol-5-yl]-3-fluoro-5-(methylsulfonyl)-4′-[2-methyl-4-(trifluoromethyl)-1H-imidazol-1-yl]biphenyl-4-yl}methanol;
(4′-(4-(1,1-difluoroethyl)-2-methyl-1H-imidazol-1-yl)-3-fluoro-5-(methylsulfonyl)-3′-(4-(4-(trifluoromethoxy)phenyl)oxazol-5-yl)biphenyl-4-yl)methanol;
N-methyl-4′-[2-[2-4-(trifluoromethyl)-1H-imidazol-1-yl]-3′-(4-{4-[(trifluoromethyl)oxy]phenyl}-1,3-oxazol-5-yl)biphenyl-3-sulfonamide;
N,N-dimethyl-4′-[2-methyl-4-(trifluoromethyl)-1H-imidazol-1-yl]-3′-(4-{4-[(trifluoromethyl)oxy]phenyl}-1,3-oxazol-5-yl)biphenyl-3-sulfonamide;
[3-fluoro-5-(methylsulfonyl)-4′-[2-methyl-4-(trifluoromethyl)-1H-imidazol-1-yl]-3′-(4-{4-[(trifluoromethyl)oxy]phenyl}-1,3-oxazol-5-yl)biphenyl-4-yl]methanol;
4-(4-chlorophenyl)-5-{3′-(methylsulfonyl)-4-[2-methyl-4-(trifluoromethyl)-1H-imidazol-1-yl]biphenyl-3-yl}-1,3-oxazole;
5-{3′-(methylsulfonyl)-4-[2-methyl-4-(trifluoromethyl)-1H-imidazol-1-yl]biphenyl-3-yl}-4-{4-[(trifluoromethyl)oxy]phenyl}-1,3-oxazole;
5-{4-[4-(1,1-difluoroethyl)-2-methyl-1H-imidazol-1-yl]-3′-(methylsulfonyl)biphenyl-3-yl}-4-{4-[(trifluoromethyl)oxy]phenyl}-1,3-oxazole;
{4′-[4-(1,1-difluoropropyl)-2-methyl-1H-imidazol-1-yl]-3-fluoro-5-(methylsulfonyl)-3′-(4-{4-[(trifluoromethyl)oxy]phenyl}-1,3-oxazol-5-yl)biphenyl-4-yl}methanol;
5-{4-[4-(1,1-difluoropropyl)-2-methyl-1H-imidazol-1-yl]-3′-(methylsulfonyl)biphenyl-3-yl}-4-{4-[(trifluoromethyl)oxy]phenyl}-1,3-oxazole;
{3′-[5-(4-chlorophenyl)-1,3-oxazol-4-yl]-3-fluoro-5-(methylsulfonyl)-4′-[2-methyl-4-(trifluoromethyl)-1H-imidazol-1-yl]biphenyl-4-yl}methanol;
2-methyl-4-{3′-(methylsulfonyl)-4-[2-methyl-4-(trifluoromethyl)-1H-imidazol-1-yl]biphenyl-3-yl}-5-{4-[(trifluoromethyl)oxy]phenyl}-1,3-oxazole;
5-(4-(difluoromethoxy)-3,5-difluorophenyl)-2-methyl-4-(4-(2-methyl-4-(trifluoromethyl)-1H-imidazol-1-yl)-3′-(methylsulfonyl)-[1,1′-biphenyl]-3-yl)oxazole;
2,2-difluoro-5-(2-methyl-4-(4-2-methyl-4-(trifluoromethyl)-1H-imidazol-1-yl)-3′-methylsulfonyl)biphenyl-3-yl)oxazol-5-yl)-2,3-dihydrobenzofuran-3-ol;
(1-(3-(5-(2,2-difluorobenzo[d][1,3]dioxol-5-yl)-2-methyloxazol-4-yl)-3′-(methylsulfonyl)biphenyl-4-yl)-2-methyl-1H-imidazol-4-yl)methanol;
2-(1-(3-(5-(2,2-difluorobenzo[d][1,3]dioxol-5-yl)-2-methyloxazol-4-yl)-3′-fluoro-4′-(hydroxymethyl)-5′-(methylsulfonyl)biphenyl-4-yl)-2-methyl-1H-imidazol-4-yl)propan-2-ol;
1-(3-(5-(2,2-difluorobenzo[d][1,3]dioxol-5-yl)-2-methyloxazol-4-yl)-3′-(methylsulfonyl)biphenyl-4-yl)-2-methyl-1H-imidazole-4-carbonitrile;
1-(1-(3-(5-(2,2-difluorobenzo[d][1,3]dioxol-5-yl)-2-methyloxazol-4-yl)-3′-(methylsulfonyl)-[1,1′-biphenyl]-4-yl)-2-methyl-1H-imidazol-4-yl)ethanol;
[3-fluoro-5-(methylsulfonyl)-4′-[2-methyl-4-(trifluoromethyl)-1H-imidazol-1-yl]-3′-(2-methyl-5-{4-[(trifluoromethyl)oxy]phenyl}-1,3-oxazol-4-yl)biphenyl-4-yl]methanol;
{3′-[5-(4-chlorophenyl)-2-methyl-1,3-oxazol-4-yl]-3-fluoro-5-(methylsulfonyl)-4′-[2-methyl-4-(trifluoromethyl)-1H-imidazol-1-yl]biphenyl-4-yl}methanol;
5-(4-chlorophenyl)-2-methyl-4-{3′-(methylsulfonyl)-4-[2-methyl-4-(trifluoromethyl)-1H-imidazol-1-yl]biphenyl-3-yl}-1,3-oxazole;
5-(4-(difluoromethoxy)-3-fluorophenyl)-2-methyl-4-(4-(2-methyl-4-(trifluoromethyl)-1H-imidazol-1-yl)-3′-(methylsulfonyl)biphenyl-3-yl)oxazole;
(3′-(5-(4-(difluoromethoxy)-3-fluorophenyl)-2-methyloxazol-4-yl)-3-fluoro-4′-(2-methyl-4-(trifluoromethyl)-1H-imidazol-1-yl)-5-(methylsulfonyl)biphenyl-4-yl)methanol;
3-(4-(3′-fluoro-4′-(hydroxymethyl)-4-(2-methyl-4-(trifluoromethyl)-1H-imidazol-1-yl)-5′-(methylsulfonyl)biphenyl-3-yl)-2-methyloxazol-5-yl)-1-isopropylpyridin-2(1H)-one;
1-isopropyl-3-(2-methyl-4-(4-(2-methyl-4-(trifluoromethyl)-1H-imidazol-1-yl)-3′-(methylsulfonyl)biphenyl-3-yl)oxazol-5-yl)pyridin-2(1H)-one;
5-(4-(difluoromethoxy)-3-methoxyphenyl)-2-methyl-4-(4-(2-methyl-4-(trifluoromethyl)-1H-imidazol-1-yl)-3′-(methylsulfonyl)biphenyl-3-yl)oxazole;
(3′-(5-(4-(difluoromethoxy)-3-methoxyphenyl)-2-methyloxazol-4-yl)-3-fluoro-4′-(2-methyl-4-(trifluoromethyl)-1H-imidazol-1-yl)-5-(methylsulfonyl)biphenyl-4-yl)methanol;
5-(3-(cyclopropylmethoxy)-4-(difluoromethoxy)phenyl)-2-methyl-4-(4-(2-methyl-4-(trifluoromethyl)-1H-imidazol-1-yl)-3′-(methylsulfonyl)biphenyl-3-yl)oxazole;
(3′-(5-(3-(cyclopropylmethoxy)-4-(difluoromethoxy)phenyl)-2-methyloxazol-4-yl)-3-fluoro-4′-(2-methyl-4-(trifluoromethyl)-1H-imidazol-1-yl)-5-(methylsulfonyl)biphenyl-4-yl)methanol;
5-(6-(difluoromethoxy)pyridin-3-yl)-2-methyl-4-(4-(2-methyl-4-(trifluoromethyl)-1H-imidazol-1-yl)-3′-(methylsulfonyl)biphenyl-3-yl)oxazole;
(3′-(5-(6-(difluoromethoxy)pyridin-3-yl)-2-methyloxazol-4-yl)-3-fluoro-4′-(2-methyl-4-(trifluoromethyl)-1H-imidazol-1-yl)-5-(methylsulfonyl)biphenyl-4-yl)methanol;
5-(3-cyclopropoxy-4-(difluoromethoxy)phenyl)-2-methyl-4-(4-(2-methyl-4-(trifluoromethyl)-1H-imidazol-1-yl)-3′-(methylsulfonyl)biphenyl-3-yl)oxazole;
(3′-(5-(3-cyclopropoxy-4-(difluoromethoxy)phenyl)-2-methyloxazol-4-yl)-3-fluoro-4′-(2-methyl-4-(trifluoromethyl)-1H-imidazol-1-yl)-5-(methylsulfonyl)biphenyl-4-yl)methanol;
5-(2-(difluoromethoxy)pyridin-3-yl)-2-methyl-4-(4-(2-methyl-4-(trifluoromethyl)-1H-imidazol-1-yl)-3′-(methylsulfonyl)biphenyl-3-yl)oxazole;
(3′-(5-(2-(difluoromethoxy)pyridin-3-yl)-2-methyloxazol-4-yl)-3-fluoro-4′-(2-methyl-4-(trifluoromethyl)-1H-imidazol-1-yl)-5-(methylsulfonyl)biphenyl-4-yl)methanol;
5-(2-cyclopropylpyridin-3-yl)-2-methyl-4-(4-(2-methyl-4-(trifluoromethyl)-1H-imidazol-1-yl)-3′-(methylsulfonyl)biphenyl-3-yl)oxazole;
(3′-(5-(2-cyclopropylpyridin-3-yl)-2-methyloxazol-4-yl)-3-fluoro-4′-(2-methyl-4-(trifluoromethyl)-1H-imidazol-1-yl)-5-(methylsulfonyl)biphenyl-4-yl)methanol;
5-(3,4-bis(difluoromethoxy)phenyl)-2-methyl-4-(4-(2-methyl-4-(trifluoromethyl)-1H-imidazol-1-yl)-3′-(methylsulfonyl)biphenyl-3-yl)oxazole;
(3′-(5-(3,4-bis(difluoromethoxy)phenyl)-2-methyloxazol-4-yl)-3-fluoro-4′-(2-methyl-4-(trifluoromethyl)-1H-imidazol-1-yl)-5-(methylsulfonyl)biphenyl-4-yl)methanol;
2-methyl-4-(4-(2-methyl-4-(trifluoromethyl)-1H-imidazol-1-yl)-3′-(methylsulfonyl)biphenyl-3-yl)-5-(2-methylpyridin-3-yl)oxazole;
(3-fluoro-4′-(2-methyl-4-(trifluoromethyl)-1H-imidazol-1-yl)-3′-(2-methyl-5-(2-methylpyridin-3-yl)oxazol-4-yl)-5-(methylsulfonyl)biphenyl-4-yl)methanol;
5-(2-chloro-4-(difluoromethoxy)phenyl)-2-methyl-4-(4-(2-methyl-4-(trifluoromethyl)-1H-imidazol-1-yl)-3′-(methylsulfonyl)biphenyl-3-yl)oxazole;
(3′-(5-(2-chloro-4-(difluoromethoxy)phenyl)-2-methyloxazol-4-yl)-3-fluoro-4′-(2-methyl-4-(trifluoromethyl)-1H-imidazol-1-yl)-5-(methylsulfonyl)biphenyl-4-yl)methanol;
5-(3-chloro-4-(difluoromethoxy)phenyl)-2-methyl-4-(4-(2-methyl-4-(trifluoromethyl)-1H-imidazol-1-yl)-3′-(methylsulfonyl)biphenyl-3-yl)oxazole;
(3′-(5-(3-chloro-4-(difluoromethoxy)phenyl)-2-methyloxazol-4-yl)-3-fluoro-4′-(2-methyl-4-(trifluoromethyl)-1H-imidazol-1-yl)-5-(methylsulfonyl)biphenyl-4-yl)methanol;
5-(5-fluoro-2-methoxypyridin-3-yl)-2-methyl-4-(4-(2-methyl-4-(trifluoromethyl)-1H-imidazol-1-yl)-3′-(methylsulfonyl)biphenyl-3-yl)oxazole;
(3-fluoro-3′-(5-(5-fluoro-2-methoxypyridin-3-yl)-2-methyloxazol-4-yl)-4′-(2-methyl-4-(trifluoromethyl)-1H-imidazol-1-yl)-5-(methylsulfonyl)biphenyl-4-yl)methanol;
(3′-(5-(4-(difluoromethoxy)-3-fluorophenyl)-2-methyloxazol-4-yl)-4′-(2-methyl-4-(trifluoromethyl)-1H-imidazol-1-yl)-3-(methylsulfonyl)biphenyl-4-yl)methanol;
5-(4-(cyclopropylmethoxy)-3-fluorophenyl)-2-methyl-4-(4-(2-methyl-4-(trifluoromethyl)-1H-imidazol-1-yl)-3′-(methylsulfonyl)biphenyl-3-yl)oxazole;
(3′-(5-(4-(cyclopropylmethoxy)-3-fluorophenyl)-2-methyloxazol-4-yl)-3-fluoro-4′-(2-methyl-4-(trifluoromethyl)-1H-imidazol-1-yl)-5-(methylsulfonyl)biphenyl-4-yl)methanol;
5-(4-(1,3-difluoropropan-2-yloxy)-3-fluorophenyl)-2-methyl-4-(4-(2-methyl-4-(trifluoromethyl)-1H-imidazol-1-yl)-3′-(methylsulfonyl)biphenyl-3-yl)oxazole;
(3′-(5-(4-(1,3-difluoropropan-2-yloxy)-3-fluorophenyl)-2-methyloxazol-4-yl)-3-fluoro-4′-(2-methyl-4-(trifluoromethyl)-1H-imidazol-1-yl)-5-(methylsulfonyl)biphenyl-4-yl)methanol;
5-(6-(cyclohexyloxy)pyridin-3-yl)-2-methyl-4-(4-(2-methyl-4-(trifluoromethyl)-1H-imidazol-1-yl)-3′-(methylsulfonyl)biphenyl-3-yl)oxazole;
(3′-(5-(6-(cyclohexyloxy)pyridin-3-yl)-2-methyloxazol-4-yl)-3-fluoro-4′-(2-methyl-4-(trifluoromethyl)-1H-imidazol-1-yl)-5-methylsulfonyl)biphenyl-4-yl)methanol;
5-(6-(cyclopentyloxy)pyridin-3-yl)-2-methyl-4-(4-(2-methyl-4-(trifluoromethyl)-1H-imidazol-1-yl)-3′-(methylsulfonyl)biphenyl-3-yl)oxazole;
(3′-(5-(6-(cyclopentyloxy)pyridin-3-yl)-2-methyloxazol-4-yl)-3-fluoro-4′-(2-methyl-4-(trifluoromethyl)-1H-imidazol-1-yl)-5-(methylsulfonyl)biphenyl-4-yl)methanol;
5-(5-methoxypyrazin-2-yl)-2-methyl-4-(4-(2-methyl-4-(trifluoromethyl)-1H-imidazol-1-yl)-3′-(methylsulfonyl)biphenyl-3-yl)oxazole;
(3-fluoro-3′-(5-(5-methoxypyrazin-2-yl)-2-methyloxazol-4-yl)-4′-(2-methyl-4-(trifluoromethyl)-1H-imidazol-1-yl)-5-(methylsulfonyl)biphenyl-4-yl)methanol;
5-(2-methoxypyrimidin-5-yl)-2-methyl-4-(4-(2-methyl-4-(trifluoromethyl)-1H-imidazol-1-yl)-3′-(methylsulfonyl)biphenyl-3-yl)oxazole;
(3-fluoro-3′-(5-(2-methoxypyrimidin-5-yl)-2-methyloxazol-4-yl)-4′-(2-methyl-4-(trifluoromethyl)-1H-imidazol-1-yl)-5-(methylsulfonyl)biphenyl-4-yl)methanol;
4-(3′-(cyclopropylsulfonyl)-4-(2-methyl-4-(trifluoromethyl)-1H-imidazol-1-yl)-[1,1′-biphenyl]-3-yl)-5-(2,2-difluorobenzo[d][1,3]dioxol-5-yl)-2-methyloxazole;
5-(2,2-difluorobenzo[d][1,3]dioxol-5-yl)-4-(3′-(difluoromethylsulfonyl)-4-(2-methyl-4-(trifluoromethyl)-1H-imidazol-1-yl)biphenyl-3-yl)-2-methyloxazole;
3′-(5-(2,2-difluorobenzo[d][1,3]dioxol-5-yl)-2-methyloxazol-4-yl)-N,N-dimethyl-4′-(2-methyl-4-(trifluoromethyl)-1H-imidazol-1-yl)biphenyl-3-carboxamide;
5-(2,2-dimethyl-2,3-dihydrobenzofuran-5-yl)-2-methyl-4-(4-(2-methyl-4-(trifluoromethyl)-1H-imidazol-1-yl)-3′-(methylsulfonyl)biphenyl-3-yl)oxazole;
3′-(5-(2,2-dimethyl-2,3-dihydrobenzofuran-5-yl)-2-methyloxazol-4-yl)-3-fluoro-4′-(2-methyl-4-(trifluoromethyl)-1H-imidazol-1-yl)-5-(methylsulfonyl)biphenyl-4-yl)methanol;
(3′-(5-(5-chloropyridin-3-yl)-2-methyloxazol-4-yl)-3-fluoro-4′-(2-methyl-4-(trifluoromethyl)-1H-imidazol-1-yl)-5-(methylsulfonyl)biphenyl-4-yl)methanol;
5-(2-chloro-6-methylpyridin-3-yl)-2-methyl-4-(4-(2-methyl-4-(trifluoromethyl)-1H-imidazol-1-yl)-3′-(methylsulfonyl)biphenyl-3-yl)oxazole;
4-(4-(4-chloro-2-methyl-1H-imidazol-1-yl)-3′-(methylsulfonyl)biphenyl-3-yl)-5-(2,2-difluorobenzo[d][1,3]dioxol-5-yl)-2-methyloxazole;
(4′-(4-chloro-2-methyl-1H-imidazol-1-yl)-3′-(5-(2,2-difluorobenzo[d][1,3]dioxol-5-yl)-2-methyloxazol-4-yl)-3-fluoro-5-(methylsulfonyl)biphenyl-4-yl)methanol;
5-(4-(2,2-difluoroethoxy)-3-fluorophenyl)-2-methyl-4-(4-(2-methyl-4-(trifluoromethyl)-1H-imidazol-1-yl)-3′-(methylsulfonyl)biphenyl-3-yl)oxazole;
(3′-(5-(4-(2,2-difluoroethoxy)-3-fluorophenyl)-2-methyloxazol-4-yl)-3-fluoro-4′-(2-methyl-4-(trifluoromethyl)-1H-imidazol-1-yl)-5-(methylsulfonyl)biphenyl-4-yl)methanol;
(3′-(5-(6-chloropyridin-2-yl)-2-methyloxazol-4-yl)-3-fluoro-4′-(2-methyl-4-(trifluoromethyl)-1H-imidazol-1-yl)-5-(methylsulfonyl)biphenyl-4-yl)methanol;
(3-fluoro-4′-(2-methyl-4-(trifluoromethyl)-1H-imidazol-1-yl)-3′-(2-methyl-5-(2-methylpyrimidin-5-yl)oxazol-4-yl)-5-(methylsulfonyl)biphenyl-4-yl)methanol;
4-(3′-(cyclopropylsulfonyl)-4-(2-methyl-4-(trifluoromethyl)-1H-imidazol-1-yl)biphenyl-3-yl)-5-(4-(difluoromethoxy)-3-fluorophenyl)-2-methyloxazole;
5-(4-(difluoromethoxy)-3-fluorophenyl)-4-(3′-(difluoromethylsulfonyl)-4-(2-methyl-4-(trifluoromethyl)-1H-imidazol-1-yl)biphenyl-3-yl)-2-methyloxazole;
5-(5-chloropyridin-2-yl)-2-methyl-4-(4-(2-methyl-4-(trifluoromethyl)-1H-imidazol-1-yl)-3′-(methylsulfonyl)biphenyl-3-yl)oxazole;
(3′-(5-(2-chloro-6-methylpyridin-4-yl)-2-methyloxazol-4-yl)-3-fluoro-4′-(2-methyl-4-(trifluoromethyl)-1H-imidazol-1-yl)-5-(methylsulfonyl)biphenyl-4-yl)methanol;
4-(3′-(cyclopropylsulfonyl)-4-(2-methyl-4-(trifluoromethyl)-1H-imidazol-1-yl)biphenyl-3-yl)-5-(2,2-difluorobenzo[d][1,3]dioxol-5-yl)-2-methyloxazole;
5-(4-chloropyridin-2-yl)-2-methyl-4-(4-(2-methyl-4-(trifluoromethyl)-1H-imidazol-1-yl)-3′-(methylsulfonyl)biphenyl-3-yl)oxazole;
5-(4-(difluoromethoxy)-3-fluorophenyl)-2-methyl-4-(4′-methyl-4-(2-methyl-4-(trifluoromethyl)-1H-imidazol-1-yl)-3′-(methylsulfonyl)biphenyl-3-yl)oxazole;
2-methyl-4-(4-(2-methyl-4-(trifluoromethyl)-1H-imidazol-1-yl)-3′-(methylsulfonyl)biphenyl-3-yl)-5-(naphthalen-2-yl)oxazole;
(3-fluoro-4′-(2-methyl-4-(trifluoromethyl)-1H-imidazol-1-yl)-3′-(2-methyl-5-(naphthalen-2-yl)oxazol-4-yl)-5-(methylsulfonyl)biphenyl-4-yl)methanol;
1-(3′-(5-(4-(difluoromethoxy)-3-fluorophenyl)-2-methyloxazol-4-yl)-4′-(2-methyl-4-(trifluoromethyl)-1H-imidazol-1-yl)biphenyl-3-yl)cyclopropanecarboxamide;
5-(5-fluoro-6-methoxypyridin-3-yl)-2-methyl-4-(4-(2-methyl-4-(trifluoromethyl)-1H-imidazol-1-yl)-3′-(methylsulfonyl)biphenyl-3-yl)oxazole;
(3-fluoro-3′-(5-(5-fluoro-6-methoxypyridin-3-yl)-2-methyloxazol-4-yl)-4′-(2-methyl-4-(trifluoromethyl)-1H-imidazol-1-yl)-5-(methylsulfonyl)biphenyl-4-yl)methanol;
5-(6-chloropyridin-2-yl)-2-methyl-4-(4-(2-methyl-4-(trifluoromethyl)-1H-imidazol-1-yl)-3′-(methylsulfonyl)biphenyl-3-yl)oxazole;
3′-(5-(4-(difluoromethoxy)-3-fluorophenyl)-2-methyloxazol-4-yl)-N,N-dimethyl-4′-(2-methyl-4-(trifluoromethyl)-1H-imidazol-1-yl)biphenyl-3-carboxamide;
5-(2,3-dihydrobenzo[b][1,4]dioxin-6-yl)-2-methyl-4-(4-(2-methyl-4-(trifluoromethyl)-1H-imidazol-1-yl)-3′-(methylsulfonyl)biphenyl-3-yl)oxazole;
3′-(5-(2,3-dihydrobenzo[b][1,4]dioxin-6-yl)-2-methyloxazol-4-yl)-3-fluoro-4′-(2-methyl-4-(trifluoromethyl)-1H-imidazol-1-yl)-5-(methylsulfonyl)biphenyl-4-yl)methanol;
5-(2,2-difluorobenzo[d][1,3]dioxol-5-yl)-2-methyl-4-(4′-methyl-4-(2-methyl-4-(trifluoromethyl)-1H-imidazol-1-yl)-3′-(methylsulfonyl)biphenyl-3-yl)oxazole;
1-(3′-(5-(2,2-difluorobenzo[d][1,3]dioxol-5-yl)-2-methyloxazol-4-yl)-4′-(2-methyl-4-(trifluoromethyl)-1H-imidazol-1-yl)biphenyl-3-yl)cyclopropanecarboxamide;
5-(3,5-difluorophenyl)-2-methyl-4-(4-(2-methyl-4-(trifluoromethyl)-1H-imidazol-1-yl)-3′-(methylsulfonyl)biphenyl-3-yl)oxazole;
(3′-(5-(3,5-difluorophenyl)-2-methyloxazol-4-yl)-3-fluoro-4′-(2-methyl-4-(trifluoromethyl)-1H-imidazol-1-yl)-5-(methylsulfonyl)biphenyl-4-yl)methanol;
5-(3-chloro-5-fluorophenyl)-2-methyl-4-(4-(2-methyl-4-(trifluoromethyl)-1H-imidazol-1-yl)-3′-(methylsulfonyl)biphenyl-3-yl)oxazole;
(3′-(5-(3-chloro-5-fluorophenyl)-2-methyloxazol-4-yl)-3-fluoro-4′-(2-methyl-4-(trifluoromethyl)-1H-imidazol-1-yl)-5-(methylsulfonyl)biphenyl-4-yl)methanol;
5-(2-chloro-6-methylpyridin-4-yl)-2-methyl-4-(4-(2-methyl-4-(trifluoromethyl)-1H-imidazol-1-yl)-3′-(methylsulfonyl)biphenyl-3-yl)oxazole;
(3′-(5-(2-chloro-6-methylpyridin-4-yl)-2-methyloxazol-4-yl)-3-fluoro-4′-(2-methyl-4-(trifluoromethyl)-1H-imidazol-1-yl)-5-(methylsulfonyl)biphenyl-4-yl)methanol;
5-(2,4-difluorophenyl)-2-methyl-4-(4-(2-methyl-4-(trifluoromethyl)-1H-imidazol-1-yl)-3′-(methylsulfonyl)biphenyl-3-yl)oxazole;
(3′-(5-(2,4-difluorophenyl)-2-methyloxazol-4-yl)-3-fluoro-4′-(2-methyl-4-(trifluoromethyl)-1H-imidazol-1-yl)-5-(methylsulfonyl)biphenyl-4-yl)methanol;
5-(3,5-dichlorophenyl)-2-methyl-4-(4-(2-methyl-4-(trifluoromethyl)-1H-imidazol-1-yl)-3′-(methylsulfonyl)biphenyl-3-yl)oxazole;
(3′-(5-(3,5-dichlorophenyl)-2-methyloxazol-4-yl)-3-fluoro-4′-(2-methyl-4-(trifluoromethyl)-1H-imidazol-1-yl)-5-(methylsulfonyl)biphenyl-4-yl)methanol;
(3′-(5-(4-(difluoromethoxy)-3,5-difluorophenyl)-2-methyloxazol-4-yl)-3-fluoro-4′-(2-methyl-4-(trifluoromethyl)-1H-imidazol-1-yl)-5-(methylsulfonyl)biphenyl-4-yl)methanol;
5-(4-chloro-3-(difluoromethoxy)phenyl)-2-methyl-4-(4-(2-methyl-4-(trifluoromethyl)-1H-imidazol-1-yl)-3′-(methylsulfonyl)biphenyl-3-yl)oxazole;
(3′-(5-(4-chloro-3-(difluoromethoxy)phenyl)-2-methyloxazol-4-yl)-3-fluoro-4′-(2-methyl-4-(trifluoromethyl)-1H-imidazol-1-yl)-5-(methylsulfonyl)biphenyl-4-yl)methanol;
5-(3-(difluoromethoxy)-4-fluorophenyl)-2-methyl-4-(4-(2-methyl-4-(trifluoromethyl)-1H-imidazol-1-yl)-3′-(methylsulfonyl)biphenyl-3-yl)oxazole;
(3′-(5-(3-(difluoromethoxy)-4-fluorophenyl)-2-methyloxazol-4-yl)-3-fluoro-4′-(2-methyl-4-(trifluoromethyl)-1H-imidazol-1-yl)-5-methylsulfonyl)biphenyl-4-yl)methanol;
5-(4-(difluoromethoxy)-3-fluorophenyl)-4-(3′-fluoro-4-(2-methyl-4-(trifluoromethyl)-1H-imidazol-1-yl)-5′-methylsulfonyl)biphenyl-3-yl)-2-methyloxazole;
5-(2,2-difluorobenzo[d][1,3]dioxol-5-yl)-4-(3′-fluoro-4-(2-methyl-4-(trifluoromethyl)-1H-imidazol-1-yl)-5′-(methylsulfonyl)biphenyl-3-yl)-2-methyloxazole;
4-(4-(4-chloro-2-methyl-1H-imidazol-1-yl)-3′-(methylsulfonyl)biphenyl-3-yl)-5-(5-fluoro-6-methoxypyridin-3-yl)-2-methyloxazole;
(4′-(4-chloro-2-methyl-1H-imidazol-1-yl)-3-fluoro-3′-(5-(5-fluoro-6-methoxypyridin-3-yl)-2-methyloxazol-4-yl)-5-(methylsulfonyl)biphenyl-4-yl)methanol;
4-(4-(4-chloro-2-methyl-1H-imidazol-1-yl)-3′-(methylsulfonyl)biphenyl-3-yl)-5-(4-(difluoromethoxy)-3-fluorophenyl)-2-methyloxazole;
(4′-(4-chloro-2-methyl-1H-imidazol-1-yl)-3′-(5-(4-(difluoromethoxy)-3-fluorophenyl)-2-methyloxazol-4-yl)-3-fluoro-5-(methylsulfonyl)biphenyl-4-yl)methanol;
4-(4-(4-chloro-2-methyl-1H-imidazol-1-yl)-3′-(methylsulfonyl)biphenyl-3-yl)-5-(2-chloro-6-methylpyridin-4-yl)-2-methyloxazole;
(3′-(5-(4-(difluoromethoxy)-3,5-difluorophenyl)-2-methyloxazol-4-yl)-4′-(2-methyl-4-(trifluoromethyl)-1H-imidazol-1-yl)-3-(methylsulfonyl)biphenyl-4-yl)methanol;
5-(4-(difluoromethoxy)-3,5-difluorophenyl)-2-methyl-4-(4′-methyl-4-(2-methyl-4-(trifluoromethyl)-1H-imidazol-1-yl)-3′-(methylsulfonyl)biphenyl-3-yl)oxazole;
5-(6-ethoxy-5-fluoropyridin-3-yl)-2-methyl-4-(4-(2-methyl-4-(trifluoromethyl)-1H-imidazol-1-yl)-3′-(methylsulfonyl)biphenyl-3-yl)oxazole;
(3′-(5-(6-ethoxy-5-fluoropyridin-3-yl)-2-methyloxazol-4-yl)-3-fluoro-4′-(2-methyl-4-(trifluoromethyl)-1H-imidazol-1-yl)-5-(methylsulfonyl)biphenyl-4-yl)methanol;
5-(6-ethoxy-5-fluoropyridin-3-yl)-2-methyl-4-(4′-methyl-4-(2-methyl-4-(trifluoromethyl)-1H-imidazol-1-yl)-3′-(methylsulfonyl)biphenyl-3-yl)oxazole;
5-(2,3-dihydro-1H-inden-5-yl)-2-methyl-4-(4-(2-methyl-4-(trifluoromethyl)-1H-imidazol-1-yl)-3′-(methylsulfonyl)biphenyl-3-yl)oxazole;
(3′-(5-(2,3-dihydro-1H-inden-5-yl)-2-methyloxazol-4-yl)-3-fluoro-4′-(2-methyl-4-(trifluoromethyl)-1H-imidazol-1-yl)-5-(methylsulfonyl)biphenyl-4-yl)methanol;
5-(2-chloro-6-(trifluoromethyl)pyridin-3-yl)-2-methyl-4-(4′-methyl-4-(2-methyl-4-(trifluoromethyl)-1H-imidazol-1-yl)-3′-(methylsulfonyl)biphenyl-3-yl)oxazole;
(3′-(5-(2-chloro-6-(trifluoromethyl)pyridin-3-yl)-2-methyloxazol-4-yl)-3-fluoro-4′-(2-methyl-4-(trifluoromethyl)-1H-imidazol-1-yl)-5-(methylsulfonyl)biphenyl-4-yl)methanol;
5-(2,2-difluorobenzo[d][1,3]dioxol-5-yl)-2-ethyl-4-(4-(2-methyl-4-(trifluoromethyl)-1H-imidazol-1-yl)-3′-(methylsulfonyl)biphenyl-3-yl)oxazole;
(3′-(5-(2,2-difluorobenzo[d][1,3]dioxol-5-yl)-2-ethyloxazol-4-yl)-3-fluoro-4′-(2-methyl-4-(trifluoromethyl)-1H-imidazol-1-yl)-5-(methylsulfonyl)biphenyl-4-yl)methanol;
5-(2,2-difluorobenzo[d][1,3]dioxol-5-yl)-2-isopropyl-4-(4-(2-methyl-4-(trifluoromethyl)-1H-imidazol-1-yl)-3′-(methylsulfonyl)biphenyl-3-yl)oxazole;
(3′-(5-(2,2-difluorobenzo[d][1,3]dioxol-5-yl)-2-isopropyloxazol-4-yl)-3-fluoro-4′-(2-methyl-4-(trifluoromethyl)-1H-imidazol-1-yl)-5-(methylsulfonyl)biphenyl-4-yl)methanol;
2-cyclopropyl-5-(2,2-difluorobenzo[d][1,3]dioxol-5-yl)-4-(4-(2-methyl-4-(trifluoromethyl)-1H-imidazol-1-yl)-3′-(methylsulfonyl)biphenyl-3-yl)oxazole;
(3′-(2-cyclopropyl-5-(2,2-difluorobenzo[d][1,3]dioxol-5-yl)oxazol-4-yl)-3-fluoro-4′-(2-methyl-4-(trifluoromethyl)-1H-imidazol-1-yl)-5-(methylsulfonyl)biphenyl-4-yl)methanol;
5-(4-chloro-2-fluorophenyl)-2-methyl-4-(4-(2-methyl-4-(trifluoromethyl)-1H-imidazol-1-yl)-3′-(methylsulfonyl)biphenyl-3-yl)oxazole;
(3′-(5-(4-chloro-2-fluorophenyl)-2-methyloxazol-4-yl)-3-fluoro-4′-(2-methyl-4-(trifluoromethyl)-1H-imidazol-1-yl)-5-(methylsulfonyl)biphenyl-4-yl)methanol;
5-(2,4-dichlorophenyl)-2-methyl-4-(4-(2-methyl-4-(trifluoromethyl)-1H-imidazol-1-yl)-3′-(methylsulfonyl)biphenyl-3-yl)oxazole;
2,2-difluoro-5-(4-(3′-fluoro-4′-(hydroxymethyl)-4-(2-methyl-4-(trifluoromethyl)-1H-imidazol-1-yl)-5′-(methylsulfonyl)biphenyl-3-yl)-2-methyloxazol-5-yl)-2,3-dihydrobenzofuran-3-ol;
(3′-(5-(2,4-dichlorophenyl)-2-methyloxazol-4-yl)-3-fluoro-4′-(2-methyl-4-(trifluoromethyl)-1H-imidazol-1-yl)-5-(methylsulfonyl)biphenyl-4-yl)methanol;
1-(3-(2-cyclopropyl-5-(2,2-difluorobenzo[d][1,3]dioxol-5-yl)oxazol-4-yl)-3′-(methylsulfonyl)biphenyl-4-yl)-2-methyl-1H-imidazole-4-carboxamide;
1-(3-(2-cyclopropyl-5-(2,2-difluorobenzo[d][1,3]dioxol-5-yl)oxazol-4-yl)-3′-fluoro-4′-(hydroxymethyl)-5′-methylsulfonyl)biphenyl-4-yl)-2-methyl-1H-imidazole-4-carboxamide;
2-methyl-4-(4-(2-methyl-4-(trifluoromethyl)-1H-imidazol-1-yl)-3′-(methylsulfonyl)-[1,1′-biphenyl]-3-yl)-5-(p-tolyl)oxazole;
(3-fluoro-4′-(2-methyl-4-(trifluoromethyl)-1H-imidazol-1-yl)-3′-(2-methyl-5-p-tolyloxazol-4-yl)-5-(methylsulfonyl)biphenyl-4-yl)methanol;
5-(2,2-difluorobenzo[d][1,3]dioxol-5-yl)-2-methyl-4-(4-(2-methyl-4-(trifluoromethyl)-1H-imidazol-1-yl)-3′-(methylsulfonyl)biphenyl-3-yl)oxazole;
(3′-(5-(2,2-difluorobenzo[d][1,3]dioxol-5-yl)-2-methyloxazol-4-yl)-3-fluoro-4′-(2-methyl-4-(trifluoromethyl)-1H-imidazol-1-yl)-5-(methylsulfonyl)biphenyl-4-yl)methanol;
5-(4-(difluoromethoxy)phenyl)-2-methyl-4-(4-(2-methyl-4-(trifluoromethyl)-1H-imidazol-1-yl)-3′-(methylsulfonyl)biphenyl-3-yl)oxazole;
(3′-(5-(4-(difluoromethoxy)phenyl)-2-methyloxazol-4-yl)-3-fluoro-4′-(2-methyl-4-(trifluoromethyl)-1H-imidazol-1-yl)-5-(methylsulfonyl)biphenyl-4-yl)methanol;
(3′-(5-(4-(difluoromethoxy)phenyl)-2-methyloxazol-4-yl)-4′-(2-methyl-4-(trifluoromethyl)-1H-imidazol-1-yl)-3-(methylsulfonyl)biphenyl-4-yl)methanol;
5-(4-chloro-3-fluorophenyl)-2-methyl-4-(4-(2-methyl-4-(trifluoromethyl)-1H-imidazol-1-yl)-3′-(methylsulfonyl)biphenyl-3-yl)oxazole;
(3′-(5-(4-chloro-3-fluorophenyl)-2-methyloxazol-4-yl)-3-fluoro-4′-(2-methyl-4-(trifluoromethyl)-1H-imidazol-1-yl)-5-methylsulfonyl)biphenyl-4-yl)methanol;
N,N-dimethyl-4-(2-methyl-4-(4-(2-methyl-4-(trifluoromethyl)-1H-imidazol-1-yl)-3′-(methylsulfonyl)biphenyl-3-yl)oxazol-5-yl)aniline;
1-(4-(4-(3′-fluoro-4′-(hydroxymethyl)-4-(2-methyl-4-(trifluoromethyl)-1H-imidazol-1-yl)-5′-(methylsulfonyl)biphenyl-3-yl)-2-methyloxazol-5-yl)phenyl)pyrrolidin-2-one;
1-(4-(2-methyl-4-(4-(2-methyl-4-(trifluoromethyl)-1H-imidazol-1-yl)-3′-(methylsulfonyl)biphenyl-3-yl)oxazol-5-yl)phenyl)pyrrolidin-2-one;
(3-fluoro-3′-(5-(3-fluoro-4-methylphenyl)-2-methyloxazol-4-yl)-4′-(2-methyl-4-(trifluoromethyl)-1H-imidazol-1-yl)-5-(methylsulfonyl)biphenyl-4-yl)methanol;
5-(3-fluoro-4-methylphenyl)-2-methyl-4-(4-(2-methyl-4-(trifluoromethyl)-1H-imidazol-1-yl)-3′-(methylsulfonyl)biphenyl-3-yl)oxazole;
5-(4-fluorophenyl)-2-methyl-4-(4-(2-methyl-4-(trifluoromethyl)-1H-imidazol-1-yl)-3′-(methylsulfonyl)biphenyl-3-yl)oxazole;
2-fluoro-5-(2-methyl-4-(4-(2-methyl-4-(trifluoromethyl)-1H-imidazol-1-yl)-3′-(methylsulfonyl)biphenyl-3-yl)oxazol-5-yl)benzonitrile;
(3′-(5-(3,4-dichlorophenyl)-2-methyloxazol-4-yl)-3-fluoro-4′-(2-methyl-4-(trifluoromethyl)-1H-imidazol-1-yl)-5-(methylsulfonyl)biphenyl-4-yl)methanol;
5-(3,4-dichlorophenyl)-2-methyl-4-(4-(2-methyl-4-(trifluoromethyl)-1H-imidazol-1-yl)-3′-(methylsulfonyl)biphenyl-3-yl)oxazole;
(3′-(5-(3-chloro-4-fluorophenyl)-2-methyloxazol-4-yl)-3-fluoro-4′-(2-methyl-4-(trifluoromethyl)-1H-imidazol-1-yl)-5-(methylsulfonyl)biphenyl-4-yl)methanol;
5-(3-chloro-4-fluorophenyl)-2-methyl-4-(4-(2-methyl-4-(trifluoromethyl)-1H-imidazol-1-yl)-3′-(methylsulfonyl)biphenyl-3-yl)oxazole;
5-(3,4-difluorophenyl)-2-methyl-4-(4-(2-methyl-4-(trifluoromethyl)-1H-imidazol-1-yl)-3′-(methylsulfonyl)biphenyl-3-yl)oxazole;
(3′-(5-(3,4-difluorophenyl)-2-methyloxazol-4-yl)-4′-(2-methyl-4-(trifluoromethyl)-1H-imidazol-1-yl)-3-(methylsulfonyl)biphenyl-4-yl)methanol;
2-methyl-4-(4-(2-methyl-4-(trifluoromethyl)-1H-imidazol-1-yl)-3′-(methylsulfonyl)biphenyl-3-yl)-5-(4-(trifluoromethyl)phenyl)oxazole;
1-(1-(3-(5-(2,2-difluorobenzo[d][1,3]dioxol-5-yl)-2-methyloxazol-4-yl)-3′-(methylsulfonyl)-[1,1′-biphenyl]-4-yl)-2-methyl-1H-imidazol-4-yl)ethanol One enantiomer
1-(1-(3-(5-(2,2-difluorobenzo[d][1,3]dioxol-5-yl)-2-methyloxazol-4-yl)-3′-fluoro-4′-(hydroxymethyl)-5′-(methylsulfonyl)-[1,1′-biphenyl]-4-yl)-2-methyl-1H-imidazol-4-yl)ethanol, one enantiomer
1-(1-(3-(5-(2,2-difluorobenzo[d][1,3]dioxol-5-yl)-2-methyloxazol-4-yl)-3′-fluoro-4′-(hydroxymethyl)-5′-(methylsulfonyl)-[1,1′-biphenyl]-4-yl)-2-methyl-1H-imidazol-4-yl)ethanol, another enantiomer
4-(4-(4-(1,1-difluoropropyl)-2-methyl-1H-imidazol-1-yl)-3′-(methylsulfonyl)-[1,1′-biphenyl]-3-yl)-2-methyl-5-(4-(trifluoromethoxy)phenyl)oxazole;
(4′-(4-(1,1-difluoropropyl)-2-methyl-1H-imidazol-1-yl)-3-fluoro-3′-(2-methyl-5-(4-(trifluoromethoxy)phenyl)oxazol-4-yl)-5-(methylsulfonyl)-[1,1′-biphenyl]-4-yl)methanol;
(3′-(2-ethyl-5-(4-(trifluoromethoxy)phenyl)oxazol-4-yl)-4′-(2-methyl-4-(trifluoromethyl)-1H-imidazol-1-yl)-3-(methylsulfonyl)-[1,1′-biphenyl]-4-yl)methanol;
4-(4-(2-methyl-4-(trifluoromethyl)-1H-imidazol-1-yl)-3′-(methylsulfonyl)-[1,1′-biphenyl]-3-yl)-2-propyl-5-(4-(trifluoromethoxy)phenyl)oxazole;
(3′-(2-isopropyl-5-(4-(trifluoromethoxy)phenyl)oxazol-4-yl)-4′-(2-methyl-4-(trifluoromethyl)-1H-imidazol-1-yl)-3-(methylsulfonyl)-[1,1′-biphenyl]-4-yl)methanol;
(4′-(2-methyl-4-(trifluoromethyl)-1H-imidazol-1-yl)-3′-(2-methyl-5-(4-(trifluoromethoxy)phenyl)oxazol-4-yl)-3-(methylsulfonyl)-[1,1′-biphenyl]-4-yl)methanol;
2-(4′-(2-methyl-4-(trifluoromethyl)-1H-imidazol-1-yl)-3′-(2-methyl-5-(4-(trifluoromethoxy)phenyl)oxazol-4-yl)-3-(methylsulfonyl)-[1,1′-biphenyl]-4-yl)ethanol;
2-(4-(4-(2-methyl-4-(trifluoromethyl)-1H-imidazol-1-yl)-3′-(methylsulfonyl)-[1,1′-biphenyl]-3-yl)-5-(4-(trifluoromethoxy)phenyl)oxazol-2-yl)ethanol;
2-(4-(3′-fluoro-4′-(hydroxymethyl)-4-(2-methyl-4-(trifluoromethyl)-1H-imidazol-1-yl)-5′-(methylsulfonyl)-[1,1′-biphenyl]-3-yl)-5-(4-(trifluoromethoxy)phenyl)oxazol-2-yl)ethanol;
2-(4-(4′-(hydroxymethyl)-4-(2-methyl-4-(trifluoromethyl)-1H-imidazol-1-yl)-3′-(methylsulfonyl)-[1,1′-biphenyl]-3-yl)-5-(4-trifluoromethoxy)phenyl)oxazol-2-yl)ethanol;
(3-fluoro-3′-(2-(2-methoxyethyl)-5-(4-(trifluoromethoxy)phenyl)oxazol-4-yl)-4′-(2-methyl-4-(trifluoromethyl)-1H-imidazol-1-yl)-5-(methylsulfonyl)-[1,1′-biphenyl]-4-yl)methanol;
2-(4-(3′-fluoro-4′-(hydroxymethyl)-4-(2-methyl-4-(trifluoromethyl)-1H-imidazol-1-yl)-5′-(methylsulfonyl)-[1,1′-biphenyl]-3-yl)-5-(4-(trifluoromethoxy)phenyl)oxazol-2-yl)-2-methylpropan-1-ol;
2-(3-fluoro-4′-(2-methyl-4-(trifluoromethyl)-1H-imidazol-1-yl)-3′-(2-methyl-5-(4-(trifluoromethoxy)phenyl)oxazol-4-yl)-5-(methylsulfonyl)-[1,1′-biphenyl]-4-yl)ethanol;
2-(5-(2,2-difluorobenzo[d][1,3]dioxol-5-yl)-4-(3′-fluoro-4′-(hydroxymethyl)-4-(2-methyl-4-(trifluoromethyl)-1H-imidazol-1-yl)-5′-(methylsulfonyl)-[1,1′-biphenyl]-3-yl)oxazol-2-yl)ethanol;
2-(5-(2,2-difluorobenzo[d][1,3]dioxol-5-yl)-4-(4-(2-methyl-4-(trifluoromethyl)-1H-imidazol-1-yl)-3′-(methylsulfonyl)-[1,1′-biphenyl]-3-yl)oxazol-2-yl)ethanol;
5-(2,2-difluorobenzo[d][1,3]dioxol-5-yl)-4-(4-(2-methyl-4-(trifluoromethyl)-1H-imidazol-1-yl)-3′-(methylsulfonyl)-[1,1′-biphenyl]-3-yl)-2-vinyloxazole;
2-methyl-4-(4-(2-methyl-4-(trifluoromethyl)-1H-imidazol-1-yl)-3′-(methylsulfinyl)-[1,1′-biphenyl]-3-yl)-5-(4-(trifluoromethoxy)phenyl)oxazole;
1-(4-(3′-fluoro-4′-(hydroxymethyl)-4-(2-methyl-4-(trifluoromethyl)-1H-imidazol-1-yl)-5′-(methylsulfonyl)-[1,1′-biphenyl]-3-yl)-5-(4-(trifluoromethoxy)phenyl)oxazol-2-yl)propan-2-ol;
1-(4-(4′-(hydroxymethyl)-4-(2-methyl-4-(trifluoromethyl)-1H-imidazol-1-yl)-3′-(methylsulfonyl)-[1,1′-biphenyl]-3-yl)-5-(4-(trifluoromethoxy)phenyl)oxazol-2-yl)propan-2-ol;
1-(4-(4-(2-methyl-4-(trifluoromethyl)-1H-imidazol-1-yl)-3′-(methylsulfonyl)-[1,1′-biphenyl]-3-yl)-5-(4-(trifluoromethoxy)phenyl)oxazol-2-yl)propan-2-ol;
2-(4-(4-(2-methyl-4-(trifluoromethyl)-1H-imidazol-1-yl)-3′-(methylsulfonyl)-[1,1′-biphenyl]-3-yl)-5-(4-(trifluoromethoxy)phenyl)oxazol-2-yl)acetic acid
N,N-dimethyl-1-(4-(4-(2-methyl-4-(trifluoromethyl)-1H-imidazol-1-yl)-3′-(methylsulfonyl)-[1,1′-biphenyl]-3-yl)-5-(4-(trifluoromethoxy)phenyl)oxazol-2-yl)methanamine;
4-(3′-((methoxymethyl)sulfonyl)-4-(2-methyl-4-(trifluoromethyl)-1H-imidazol-1-yl)-[1,1′-biphenyl]-3-yl)-2-methyl-5-(4-(trifluoromethoxy)phenyl)oxazole;
3′-(5-(4-(difluoromethoxy)phenyl)-2-methyloxazol-4-yl)-N-methyl-4′-(2-methyl-4-(trifluoromethyl)-1H-imidazol-1-yl)-[1,1′-biphenyl]-3-sulfonamide;
3′-(5-(4-(difluoromethoxy)phenyl)-2-methyloxazol-4-yl)-N,N-dimethyl-4′-(2-methyl-4-(trifluoromethyl)-1H-imidazol-1-yl)-[1,1′-biphenyl]-3-sulfonamide;
2-(3′-(5-(4-(difluoromethoxy)phenyl)-2-methyloxazol-4-yl)-4′-(2-methyl-4-(trifluoromethyl)-1H-imidazol-1-yl)-[1,1′-biphenyl]-3-yl)-2-methylpropanamide;
(3-fluoro-4′-(2-methyl-4-(trifluoromethyl)-1H-imidazol-1-yl)-3′-(2-methyl-5-(4-((trifluoromethyl)thio)phenyl)oxazol-4-yl)-5-(methylsulfonyl)-[1,1′-biphenyl]-4-yl)methanol;
(3-fluoro-4′-(2-methyl-4-(trifluoromethyl)-1H-imidazol-1-yl)-3′-(2-methyl-5-(4-(1,1,2,2-tetrafluoroethoxy)phenyl)oxazol-4-yl)-5-(methylsulfonyl)-[1,1′-biphenyl]-4-yl)methanol;
(3-fluoro-4′-(2-methyl-4-(trifluoromethyl)-1H-imidazol-1-yl)-3′-(2-methyl-5-(3-(trifluoromethoxy)phenyl)oxazol-4-yl)-5-(methylsulfonyl)-[1,1′-biphenyl]-4-yl)methanol;
(3-fluoro-4′-(2-methyl-4-(trifluoromethyl)-1H-imidazol-1-yl)-3′-(2-methyl-5-(3-(1,1,2,2-tetrafluoroethoxy)phenyl)oxazol-4-yl)-5-(methylsulfonyl)-[1,1′-biphenyl]-4-yl)methanol;
5-(2-(difluoromethoxy)phenyl)-2-methyl-4-(4-(2-methyl-4-(trifluoromethyl)-1H-imidazol-1-yl)-3′-(methylsulfonyl)-[1,1′-biphenyl]-3-yl)oxazole;
(3′-(5-(2-(difluoromethoxy)phenyl)-2-methyloxazol-4-yl)-3-fluoro-4′-(2-methyl-4-(trifluoromethyl)-1H-imidazol-1-yl)-5-(methylsulfonyl)-[1,1′-biphenyl]-4-yl)methanol;
(3′-(5-(2-chloropyridin-3-yl)-2-methyloxazol-4-yl)-3-fluoro-4′-(2-methyl-4-(trifluoromethyl)-1H-imidazol-1-yl)-5-(methylsulfonyl)-[1,1′-biphenyl]-4-yl)methanol;
5-(6-chloropyridin-3-yl)-2-methyl-4-(4-(2-methyl-4-(trifluoromethyl)-1H-imidazol-1-yl)-3′-(methylsulfonyl)-[1,1′-biphenyl]-3-yl)oxazole;
(3′-(5-(6-chloropyridin-3-yl)-2-methyloxazol-4-yl)-3-fluoro-4′-(2-methyl-4-(trifluoromethyl)-1H-imidazol-1-yl)-5-(methylsulfonyl)-[1,1′-biphenyl]-4-yl)methanol;
5-(2-chloropyridin-3-yl)-2-methyl-4-(4-(2-methyl-4-(trifluoromethyl)-1H-imidazol-1-yl)-3′-(methylsulfonyl)-[1,1′-biphenyl]-3-yl)oxazole;
5-(2,4-dichlorophenyl)-2-(2-methoxyethyl)-4-(4-(2-methyl-4-(trifluoromethyl)-1H-imidazol-1-yl)-3′-(methylsulfonyl)-[1,1′-biphenyl]-3-yl)oxazole;
2-(4-(3′-fluoro-4′-(hydroxymethyl)-4-(2-methyl-4-(trifluoromethyl)-1H-imidazol-1-yl)-5′-(methylsulfonyl)-[1,1′-biphenyl]-3-yl)-5-(4-(trifluoromethoxy)phenyl)oxazol-2-yl)propan-1-ol;
2-ethyl-4-(4-(2-methyl-4-(trifluoromethyl)-1H-imidazol-1-yl)-3′-(methylsulfonyl)-[1,1′-biphenyl]-3-yl)-5-(4-(trifluoromethoxy)phenyl)oxazole;
(3′-(2-ethyl-5-(4-(trifluoromethoxy)phenyl)oxazol-4-yl)-3-fluoro-4′-(2-methyl-4-(trifluoromethyl)-1H-imidazol-1-yl)-5-(methylsulfonyl)-[1,1′-biphenyl]-4-yl)methanol;
(3-fluoro-4′-(2-methyl-4-(trifluoromethyl)-1H-imidazol-1-yl)-5-(methylsulfonyl)-3′-(2-propyl-5-(4-(trifluoromethoxy)phenyl)oxazol-4-yl)-[1,1′-biphenyl]-4-yl)methanol;
(4′-(2-methyl-4-(trifluoromethyl)-1H-imidazol-1-yl)-3-(methylsulfonyl)-3′-(2-propyl-5-(4-(trifluoromethoxy)phenyl)oxazol-4-yl)-[1,1′-biphenyl]-4-yl)methanol;
2-isopropyl-4-(4-(2-methyl-4-(trifluoromethyl)-1H-imidazol-1-yl)-3′-(methylsulfonyl)-[1,1′-biphenyl]-3-(trifluoromethoxy)phenyl)oxazole;
(3-fluoro-3′-(2-isopropyl-5-(4-(trifluoromethoxy)phenyl)oxazol-4-yl)-4′-(2-methyl-4-(trifluoromethyl)-1H-imidazol-1-yl)-5-(methylsulfonyl)-[1,1′-biphenyl]-4-yl)methanol;
(3-fluoro-3′-(2-isobutyl-5-(4-(trifluoromethoxy)phenyl)oxazol-4-yl)-4′-(2-methyl-4-(trifluoromethyl)-1H-imidazol-1-yl)-5-(methylsulfonyl)-[1,1′-biphenyl]-4-yl)methanol;
2-isobutyl-4-(4-(2-methyl-4-(trifluoromethyl)-1H-imidazol-1-yl)-3′-(methylsulfonyl)-[1,1′-biphenyl]-3-yl)-5-(4-(trifluoromethoxy)phenyl)oxazole;
(3′-(2-isobutyl-5-(4-(trifluoromethoxy)phenyl)oxazol-4-yl)-4′-(2-methyl-4-(trifluoromethyl)-1H-imidazol-1-yl)-3-(methylsulfonyl)-[1,1′-biphenyl]-4-yl)methanol;
2-(2-methoxyethyl)-4-(4-(2-methyl-4-(trifluoromethyl)-1H-imidazol-1-yl)-3′-(methylsulfonyl)-[1,1′-biphenyl]-3-yl)-5-(4-(trifluoromethoxy)phenyl)oxazole;
2-(4-(4-(2-methyl-4-(trifluoromethyl)-1H-imidazol-1-yl)-3′-(methylsulfonyl)-[1,1′-biphenyl]-3-yl)-5-(4-(trifluoromethoxy)phenyl)oxazol-2-yl)propan-1-ol;
5-(2,6-dichloropyridin-3-yl)-2-methyl-4-(4-(2-methyl-4-(trifluoromethyl)-1H-imidazol-1-yl)-3′-(methylsulfonyl)-[1,1′-biphenyl]-3-yl)oxazole;
(3′-(5-(2,6-dichloropyridin-3-yl)-2-methyloxazol-4-yl)-3-fluoro-4′-(2-methyl-4-(trifluoromethyl)-1H-imidazol-1-yl)-5-(methylsulfonyl)-[1,1′-biphenyl]-4-yl)methanol;
(3-fluoro-4′-(2-methyl-4-(trifluoromethyl)-1H-imidazol-1-yl)-3′-(2-methyl-5-(6-(trifluoromethyl)pyridin-3-yl)oxazol-4-yl)-5-(methylsulfonyl)-[1,1′-biphenyl]-4-yl)methanol;
5-(6-methoxypyridin-3-yl)-2-methyl-4-(4-(2-methyl-4-(trifluoromethyl)-1H-imidazol-1-yl)-3′-(methylsulfonyl)-[1,1′-biphenyl]-3-yl)oxazole;
(3-fluoro-3′-(5-(6-methoxypyridin-3-yl)-2-methyloxazol-4-yl)-4′-(2-methyl-4-(trifluoromethyl)-1H-imidazol-1-yl)-5-(methylsulfonyl)-[1,1′-biphenyl]-4-yl)methanol;
2-methyl-4-(4-(2-methyl-4-(trifluoromethyl)-1H-imidazol-1-yl)-3′-(methylsulfonyl)-[1,1′-biphenyl]-3-yl)-5-(4-((trifluoromethyl)thio)phenyl)oxazole;
2-methyl-4-(4-(2-methyl-4-(trifluoromethyl)-1H-imidazol-1-yl)-3′-(methylsulfonyl)-[1,1′-biphenyl]-3-yl)-5-(4-(perfluoroethoxy)phenyl)oxazole;
2-methyl-4-(4-(2-methyl-4-(trifluoromethyl)-1H-imidazol-1-yl)-3′-(methylsulfonyl)-[1,1′-biphenyl]-3-yl)-5-(3-(trifluoromethoxy)phenyl)oxazole;
2-methyl-4-(4-(2-methyl-4-(trifluoromethyl)-1H-imidazol-1-yl)-3′-(methylsulfonyl)-[1,1′-biphenyl]-3-yl)-5-(3-(perfluoroethoxy)phenyl)oxazole;
{3′-(2-ethyl-4-{4-[(trifluoromethyl)oxy]phenyl}-1,3-oxazol-5-yl)-3-fluoro-5-(methylsulfonyl)-4′-[2-methyl-4-(trifluoromethyl)-1H-imidazol-1-yl]biphenyl-4-yl}methanol;
{3-fluoro-3′-[2-(1-methylethyl)-4-{4-[(trifluoromethyl)oxy]phenyl}-1,3-oxazol-5-yl]-5-(methylsulfonyl)-4′-[2-methyl-4-(trifluoromethyl)-1H-imidazol-1-yl]biphenyl-4-yl}methanol;
5-{3′-(methylsulfonyl)-4-[2-methyl-4-(trifluoromethyl)-1H-imidazol-1-yl]biphenyl-3-yl}-2-propyl-4-{4-[(trifluoromethyl)oxy]phenyl}-1,3-oxazole;
(3-fluoro-3′-(2-methyl-4-(4-(trifluoromethoxy)phenyl)oxazol-5-yl)-4′-(2-methyl-4-(trifluoromethyl)-1H-imidazol-1-yl)-5-(methylsulfonyl)-[1,1′-biphenyl]-4-yl)methanol;
2-methyl-5-(4-(2-methyl-4-(trifluoromethyl)-1H-imidazol-1-yl)-3′-(methylsulfonyl)-[1,1′-biphenyl]-3-(trifluoromethoxy)phenyl)oxazole BMS-930815/EXEL-04613389;
4-(4-chlorophenyl)-2-methyl-5-(4-(2-methyl-4-(trifluoromethyl)-1H-imidazol-1-yl)-3′-(methylsulfonyl)-[1,1′-biphenyl]-3-yl)oxazole;
(3′-(4-(4-chlorophenyl)-2-methyloxazol-5-yl)-3-fluoro-4′-(2-methyl-4-(trifluoromethyl)-1H-imidazol-1-yl)-5-(methylsulfonyl)-[1,1′-biphenyl]-4-yl)methanol;
(3′-(2-methyl-4-(4-(trifluoromethoxy)phenyl)oxazol-5-yl)-4′-(2-methyl-4-(trifluoromethyl)-1H-imidazol-1-yl)-3-(methylsulfonyl)-[1,1′-biphenyl]-4-yl)methanol;
4-(2,4-dichlorophenyl)-2-methyl-5-(4-(2-methyl-4-(trifluoromethyl)-1H-imidazol-1-yl)-3′-(methylsulfonyl)-[1,1′-biphenyl]-3-yl)oxazole;
(3′-(4-(2,4-dichlorophenyl)-2-methyloxazol-5-yl)-3-fluoro-4′-(2-methyl-4-(trifluoromethyl)-1H-imidazol-1-yl)-5-(methylsulfonyl)-[1,1′-biphenyl]-4-yl)methanol;
2-(5-(4-(2-methyl-4-(trifluoromethyl)-1H-imidazol-1-yl)-3′-(methylsulfonyl)-[1,1′-biphenyl]-3-yl)-4-(4-(trifluoromethoxy)phenyl)oxazol-2-yl)acetic acid
2-(5-(4-(2-methyl-4-(trifluoromethyl)-1H-imidazol-1-yl)-3′-(methylsulfonyl)-[1,1′-biphenyl]-3-yl)-4-(4-(trifluoromethoxy)phenyl)oxazol-2-yl)ethanol;
2-(5-(3′-fluoro-4′-(hydroxymethyl)-4-(2-methyl-4-(trifluoromethyl)-1H-imidazol-1-yl)-5′-(methylsulfonyl)-[1,1′-biphenyl]-3-yl)-4-(4-(trifluoromethoxy)phenyl)oxazol-2-yl)ethanol;
2-(5-(4′-(hydroxymethyl)-4-(2-methyl-4-(trifluoromethyl)-1H-imidazol-1-yl)-3′-(methylsulfonyl)-[1,1′-biphenyl]-3-yl)-4-(4-(trifluoromethoxy)phenyl)oxazol-2-yl)ethanol;
(3′-(4-(2,4-dichlorophenyl)-2-methyloxazol-5-yl)-4′-(2-methyl-4-(trifluoromethyl)-1H-imidazol-1-yl)-3-(methylsulfonyl)-[1,1′-biphenyl]-4-yl)methanol;
4-(2,2-difluorobenzo[d][1,3]dioxol-5-yl)-2-methyl-5-(4-(2-methyl-4-(trifluoromethyl)-1H-imidazol-1-yl)-3′-(methylsulfonyl)-[1,1′-biphenyl]-3-yl)oxazole;
(3′-(4-(2,2-difluorobenzo[d][1,3]dioxol-5-yl)-2-methyloxazol-5-yl)-4′-(2-methyl-4-(trifluoromethyl)-1H-imidazol-1-yl)-3-(methylsulfonyl)-[1,1′-biphenyl]-4-yl)methanol;
(3′-(4-(2,2-difluorobenzo[d][1,3]dioxol-5-yl)-2-methyloxazol-5-yl)-3-fluoro-4′-(2-methyl-4-(trifluoromethyl)-1H-imidazol-1-yl)-5-(methylsulfonyl)-[1,1′-biphenyl]-4-yl)methanol;
2-(4-(4-chlorophenyl)-5-(4-(2-methyl-4-(trifluoromethyl)-1H-imidazol-1-yl)-3′-(methylsulfonyl)-[1,1′-biphenyl]-3-yl)oxazol-2-yl)ethanol;
4-(4-(difluoromethoxy)phenyl)-2-methyl-5-(4-(2-methyl-4-(trifluoromethyl)-1H-imidazol-1-yl)-3′-(methylsulfonyl)-[1,1′-biphenyl]-3-yl)oxazole;
5-(4-(2-methyl-4-(trifluoromethyl)-1H-imidazol-1-yl)-3′-(methylsulfonyl)-[1,1′-biphenyl]-3-yl)-2-(2-(pyrrolidin-1-yl)ethyl)-4-(4-(trifluoromethoxy)phenyl)oxazole;
2-(4-(2,2-difluorobenzo[d][1,3]dioxol-5-yl)-5-(3′-fluoro-4′-(hydroxymethyl)-4-(2-methyl-4-(trifluoromethyl)-1H-imidazol-1-yl)-5′-(methylsulfonyl)-[1,1′-biphenyl]-3-yl)oxazol-2-yl)ethanol;
2-(4-(2,2-difluorobenzo[d][1,3]dioxol-5-yl)-5-(4-(2-methyl-4-(trifluoromethyl)-1H-imidazol-1-yl)-3′-(methylsulfonyl)-[1,1′-biphenyl]-3-yl)oxazol-2-yl)ethanol;
(3-chloro-3′-(4-(2,2-difluorobenzo[d][1,3]dioxol-5-yl)-2-methyloxazol-5-yl)-4′-(2-methyl-4-(trifluoromethyl)-1H-imidazol-1-yl)-5-(methylsulfonyl)-[1,1′-biphenyl]-4-yl)methanol;
(3′-(4-(4-(difluoromethoxy)phenyl)-2-methyloxazol-5-yl)-3-fluoro-4′-(2-methyl-4-(trifluoromethyl)-1H-imidazol-1-yl)-5-(methylsulfonyl)-[1,1′-biphenyl]-4-yl)methanol;
(3′-(4-(4-(difluoromethoxy)phenyl)-2-methyloxazol-5-yl)-4′-(2-methyl-4-(trifluoromethyl)-1H-imidazol-1-yl)-3-(methylsulfonyl)-[1,1′-biphenyl]-4-yl)methanol;
2-(2-methyl-5-(4-(2-methyl-4-(trifluoromethyl)-1H-imidazol-1-yl)-3′-(methylsulfonyl)-[1,1′-biphenyl]-3-yl)oxazol-4-yl)-5-(trifluoromethoxy)phenol;
2-methyl-5-(4-(2-methyl-4-(trifluoromethyl)-1H-imidazol-1-yl)-3′-(methylsulfinyl)-[1,1′-biphenyl]-3-yl)-4-(4-(trifluoromethoxy)phenyl)oxazole;
N,N-dimethyl-2-(5-(4-(2-methyl-4-(trifluoromethyl)-1H-imidazol-1-yl)-3′-(methylsulfonyl)-[1,1′-biphenyl]-3-(trifluoromethoxy)phenyl)oxazol-2-yl)ethanamine;
2-methyl-5-(4-(2-methyl-4-(trifluoromethyl)-1H-imidazol-1-yl)-3′-(methylsulfonyl)-[1,1′-biphenyl]-3-yl)-4-(2-(trifluoromethyl)phenyl)oxazole;
(3-fluoro-3′-(2-methyl-4-(2-(trifluoromethyl)phenyl)oxazol-5-yl)-4′-(2-methyl-4-(trifluoromethyl)-1H-imidazol-1-yl)-5-(methylsulfonyl)-[1,1′-biphenyl]-4-yl)methanol;
2-methyl-5-(4-(2-methyl-4-(trifluoromethyl)-1H-imidazol-1-yl)-3′-(methylsulfonyl)-[1,1′-biphenyl]-3-yl)-4-(2-(trifluoromethoxy)phenyl)oxazole;
(3-fluoro-3′-(2-methyl-4-(2-(trifluoromethoxy)phenyl)oxazol-5-yl)-4′-(2-methyl-4-(trifluoromethyl)-1H-imidazol-1-yl)-5-(methylsulfonyl)-[1,1′-biphenyl]-4-yl)methanol;
(3′-(4-(4-chlorobenzyl)oxazol-5-yl)-3-fluoro-4′-(2-methyl-4-(trifluoromethyl)-1H-imidazol-1-yl)-5-(methylsulfonyl)-[1,1′-biphenyl]-4-yl)methanol;
4-(4-chlorobenzyl)-5-(4-(2-methyl-4-(trifluoromethyl)-1H-imidazol-1-yl)-3′-(methylsulfonyl)-[1,1′-biphenyl]-3-yl)oxazole;
(3′-(4-(4-chlorobenzyl)oxazol-5-yl)-4′-(2-methyl-4-(trifluoromethyl)-1H-imidazol-1-yl)-3-(methylsulfonyl)-[1,1′-biphenyl]-4-yl)methanol;
5-(4-(2-methyl-4-(trifluoromethyl)-1H-imidazol-1-yl)-3′-(methylsulfonyl)-[1,1′-biphenyl]-3-yl)-4-(4-(trifluoromethoxy)benzyl)oxazole;
4-(2-methoxy-4-(trifluoromethoxy)phenyl)-2-methyl-5-(4-(2-methyl-4-(trifluoromethyl)-1H-imidazol-1-yl)-3′-(methylsulfonyl)-[1,1′-biphenyl]-3-yl)oxazole;
(3′-(1-(2,2-difluorobenzo[d][1,3]dioxol-5-yl)-4-(trifluoromethyl)-1H-imidazol-2-yl)-3-fluoro-4′-(2-methyl-4-(trifluoromethyl)-1H-imidazol-1-yl)-5-methylsulfonyl)biphenyl-4-yl)methanol;
(3′-(4-cyclopropyl-1-(2,2-difluorobenzo[d][1,3]dioxol-5-yl)-1H-imidazol-2-yl)-3-fluoro-4′-(2-methyl-4-(trifluoromethyl)-1H-imidazol-1-yl)-5-(methylsulfonyl)biphenyl-4-yl)methanol;
1-(2,2-difluorobenzo[d][1,3]dioxol-5-yl)-2-(4-(2-methyl-4-(trifluoromethyl)-1H-imidazol-1-yl)-3′-(methylsulfonyl)biphenyl-3-yl)-4-(trifluoromethyl)-1H-imidazole;
1-(2,2-difluorobenzo[d][1,3]dioxol-5-yl)-2-(3′-fluoro-4-(2-methyl-4-(trifluoromethyl)-1H-imidazol-1-yl)-5′-(methylsulfonyl)biphenyl-3-yl)-4-(trifluoromethyl)-1H-imidazole;
2-chloro-5-(2-(4-(2-methyl-4-(trifluoromethyl)-1H-imidazol-1-yl)-3′-(methylsulfonyl)biphenyl-3-yl)-4-(trifluoromethyl)-1H-imidazol-1-yl)pyridine;
4-cyclopropyl-1-(2,2-difluorobenzo[d][1,3]dioxol-5-yl)-2-(4-(2-methyl-4-(trifluoromethyl)-1H-imidazol-1-yl)-3′-(methylsulfonyl)biphenyl-3-yl)-1H-imidazole;
1-(4-chlorophenyl)-2-(4-(2-methyl-4-(trifluoromethyl)-1H-imidazol-1-yl)-3′-(methylsulfonyl)biphenyl-3-yl)-4-(trifluoromethyl)-1H-imidazole;
(3′-(1-(4-chlorophenyl)-4-(trifluoromethyl)-1H-imidazol-2-yl)-3-fluoro-4′-(2-methyl-4-(trifluoromethyl)-1H-imidazol-1-yl)-5-(methylsulfonyl)biphenyl-4-yl)methanol;
1-(4-chlorophenyl)-4-cyclopropyl-2-(4-(2-methyl-4-(trifluoromethyl)-1H-imidazol-1-yl)-3′-(methylsulfonyl)-[1,1′-biphenyl]-3-yl)-1H-imidazole;
(3′-(1-(4-chlorophenyl)-4-cyclopropyl-1H-imidazol-2-yl)-3-fluoro-4′-(2-methyl-4-(trifluoromethyl)-1H-imidazol-1-yl)-5-(methylsulfonyl)-[1,1′-biphenyl]-4-yl)methanol;
or a pharmaceutically acceptable salt thereof.
In another aspect, the compound is a compound described in U.S. Pat. No. 8,618,154 and is selected from the group:
1 2-(1-(3′-fluoro-4′-(hydroxymethyl)-5′-(methylsulfonyl)biphenyl-4-yl)-2-(2-(2-fluorophenyl)propan-2-yl)-1H-imidazol-4-yl)propan-2-ol;
2 2-(2-(2-(2-chloro-6-fluorophenyl)propan-2-yl)-1-(3′-fluoro-4′-(hydroxymethyl)-3-methyl-5′-(methylsulfonyl)biphenyl-4-yl)-1H-imidazol-4-yl)propan-2-ol;
3 2-(1-(3-chloro-3′-fluoro-4′-(hydroxymethyl)-5′-(methylsulfonyl)bi-phenyl-4-yl)-2-(2-(2,6-dichlorophenyl)propan-2-yl)-1H-imidazol-4-yl)propan-2-ol;
4 2-(2-(2-(2-chloro-3-fluorophenyl)propan-2-yl)-1-(3′-fluoro-4′-(hydroxymethyl)-5′-(methylsulfonyl)biphenyl-4-yl)-1H-imidazol-4-yl)propan-2-ol;
5 2-(2-(2-(2,6-dichlorophenyl)propan-2-yl)-1-(3′-fluoro-4′-(hydroxy-methyl)-5′-(methylsulfonyl)biphenyl-4-yl)-1H-imidazol-4-yl)propan-2-ol;
6 2-(2-(2-(2-Chloro-phenyl)propan-2-yl)-1-(3,3′-difluoro-4′-hydroxy-methyl-5′-(methylsulfonyl)biphenyl-4-yl)-1H-imidazol-4-yl)-propan-2-ol;
7 2-(2-(2-(2-chloro-6-fluorophenyl)propan-2-yl)-1-(3,3′-difluoro-4′-(hydroxymethyl)-5′-(methylsulfonyl)biphenyl-4-yl)-1H-imidazol-4-yl)propan-2-ol;
8 2-{1-(3,3′-Difluoro-4′-hydroxymethyl-5′-methanesulfonyl-biphenyl-4-yl)-2-[2-(2-fluorophenyl)propan-2-yl]-1H-imidazol-4-yl}-propan-2-ol;
9 2-(2-(2-(2,6-dichlorophenyl)propan-2-yl)-1-(3,3′-difluoro-4′-(hydroxymethyl)-5′-(methylsulfonyl)biphenyl-4-yl)-1H-imidazol-4-yl)propan-2-ol;
10 2-(2-(2-(2,6-dichlorophenyl)propan-2-yl)-1-(3,3′-difluoro-4′-(hydroxymethyl)-5′-(methylsulfonyl)biphenyl-4-yl)-1H-imidazol-4-yl)[(¹³CD₃)₂]propan-2-ol;
11 2-(2-(2,4-dichlorobenzyl)-1-(3,3′-difluoro-4′-(hydroxymethyl)-5′-(methylsulfonyl)biphenyl-4-yl)-1H-imidazol-4-yl)propan-2-ol;
12 2-(1-(3,3′-difluoro-4′-(hydroxymethyl)-5′-(methylsulfonyl)biphenyl-4-yl)-2-(2-(trifluoromethyl)benzyl)-1H-imidazol-4-yl)propan-2-ol;
13 2-(1-(3-chloro-3′-fluoro-4′-(hydroxymethyl)-5′-(methylsulfonyl)bi-phenyl-4-yl)-2-(2-chloro-4-fluorobenzyl)-1H-imidazol-4-yl)propan-2-ol;
14 2-(2-(2-chloro-4-fluorobenzyl)-1-(3,3′-difluoro-4′-(hydroxymethyl)-5′-(methylsulfonyl)biphenyl-4-yl)-1H-imidazol-4-yl)propan-2-ol;
15 2-(2-(2,4-dichlorobenzyl)-1-(3′-fluoro-4′-(hydroxymethyl)-5′-(methylsulfonyl)biphenyl-4-yl)-1H-imidazol-4-yl)propan-2-ol;
16 2-(1-(3,3′-difluoro-4′-(hydroxymethyl)-5′-(methylsulfonyl)biphenyl-4-yl)-2-(2-fluorobenzyl)-1H-imidazol-4-yl)propan-2-ol;
17 2-(1-(3′-fluoro-4′-(hydroxymethyl)-5′-(methylsulfonyl)biphenyl-4-yl)-2-(2-methylbenzyl)-1H-imidazol-4-yl)propan-2-ol;
18 2-(2-(2,6-dichlorobenzyl)-1-(3′-fluoro-4′-(hydroxymethyl)-5′-(methylsulfonyl)biphenyl-4-yl)-1H-imidazol-4-yl)propan-2-ol;
19 2-[2-(2-Chloro-5-fluoro-benzyl)-1-(3′-fluoro-4′-hydroxymethyl-5′-methanesulfonyl-biphenyl-4-yl)-1H-imidazol-4-yl]-propan-2-ol;
20 2-[2-(2-Chloro-benzyl)-1-(3,3′-difluoro-4′-hydroxymethyl-5′-methanesulfonyl-biphenyl-4-yl)-1H-imidazol-4-yl]-propan-2-ol;
and 21 2-{2-[1-(2,6-dichlorophenyl)ethyl]-1-[3,3′-difluoro-4′-(hydroxy-methyl)-5′-(methylsulfonyl)biphenyl-4-yl]-1H-imidazol-4-yl}propan-2-ol;
   or a pharmaceutically acceptable salt thereof.

### Examples of Inhibitor Compounds

In another aspect, the compound is selected from fenofibrate, morin, and luteolin.

### LXR Inverse Agonists

In another aspect, the compound is:

SR9243

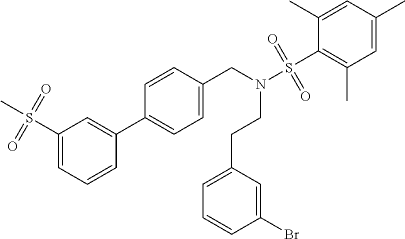

or a pharmaceutically acceptable salt thereof.

In another aspect, the compound is: TLC-2716 or a pharmaceutically acceptable salt thereof.

In another aspect, the compound is:

SR9238

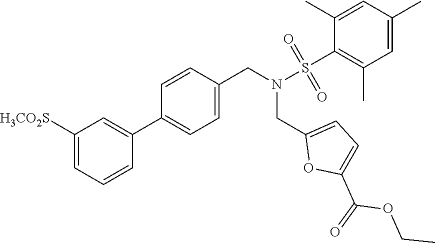

or a pharmaceutically acceptable salt thereof.

In another aspect, the compound is a compound described in US 20200308135 (see Formula (I) below).

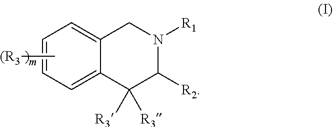

In another aspect, the compound is selected from the group:

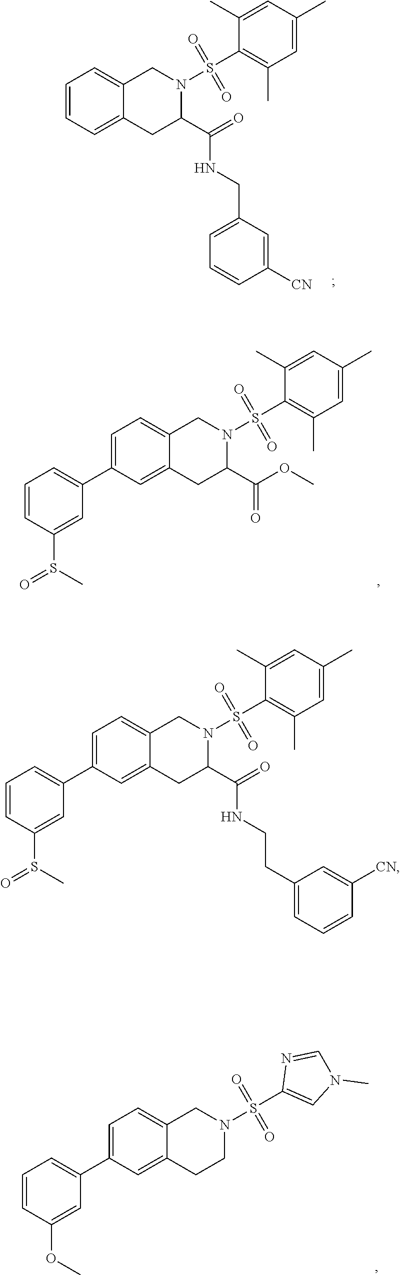 [SMILES: Cc1cc(C)c(S(=O)(=O)N2Cc3ccccc3CC2C(=O)NCc2cccc(C#N)c2)c(C)c1 ; COC(=O)C1Cc2cc(-c3cccc(S(C)=O)c3)ccc2CN1S(=O)(=O)c1c(C)cc(C)cc1C ; Cc1cc(C)c(S(=O)(=O)N2Cc3ccc(-c4cccc(S(C)=O)c4)cc3CC2C(=O)NCCc2cccc(C#N)c2)c(C)c1 ; COc1cccc(-c2ccc3c(c2)CCN(S(=O)(=O)c2cn(C)cn2)C3)c1]

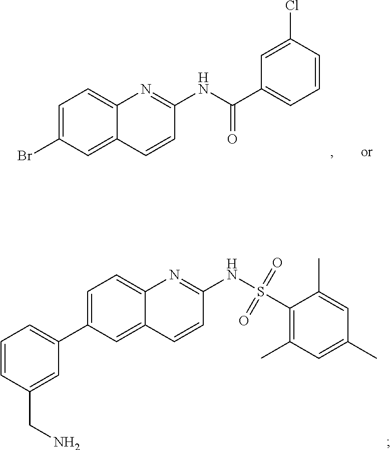 [SMILES: O=C(Nc1ccc2cc(Br)ccc2n1)c1cccc(Cl)c1 ; Cc1cc(C)c(S(=O)(=O)Nc2ccc3cc(-c4cccc(CN)c4)ccc3n2)c(C)c1]

or a pharmaceutically acceptable salt thereof.

In another aspect, the compound is a spiro(3,3′-isopropylpyrrolidine oxindole) described in US 20220135579A1 (see Formula (I) below).

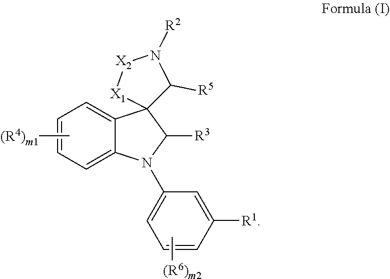

In another aspect, the compound is selected from the group:

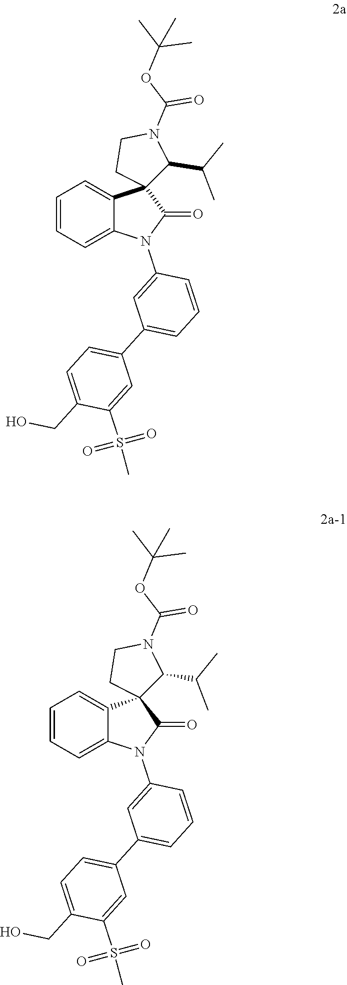

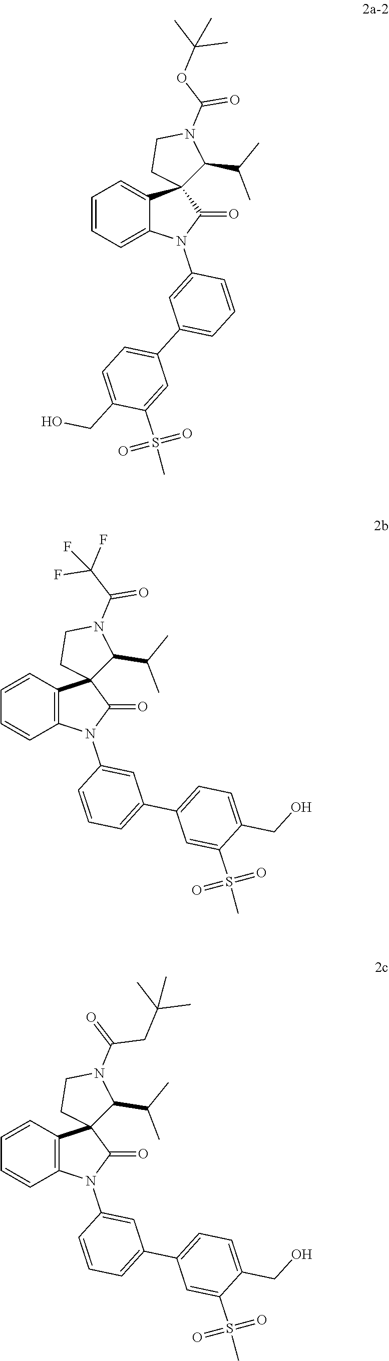 [SMILES: CC(C)[C@@H]1N(C(=O)OC(C)(C)C)CC[C@@]12C(=O)N(c1cccc(-c3ccc(CO)c(S(C)(=O)=O)c3)c1)c1ccccc12 ; CC(C)[C@@H]1N(C(=O)C(F)(F)F)CC[C@@]12C(=O)N(c1cccc(-c3ccc(CO)c(S(C)(=O)=O)c3)c1)c1ccccc12 ; CC(C)[C@@H]1N(C(=O)CC(C)(C)C)CC[C@@]12C(=O)N(c1cccc(-c3ccc(CO)c(S(C)(=O)=O)c3)c1)c1ccccc12]

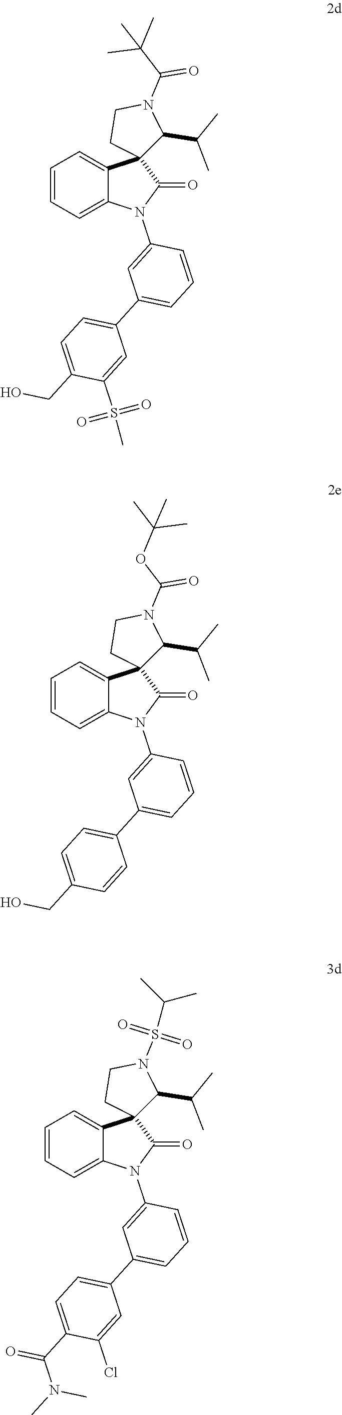

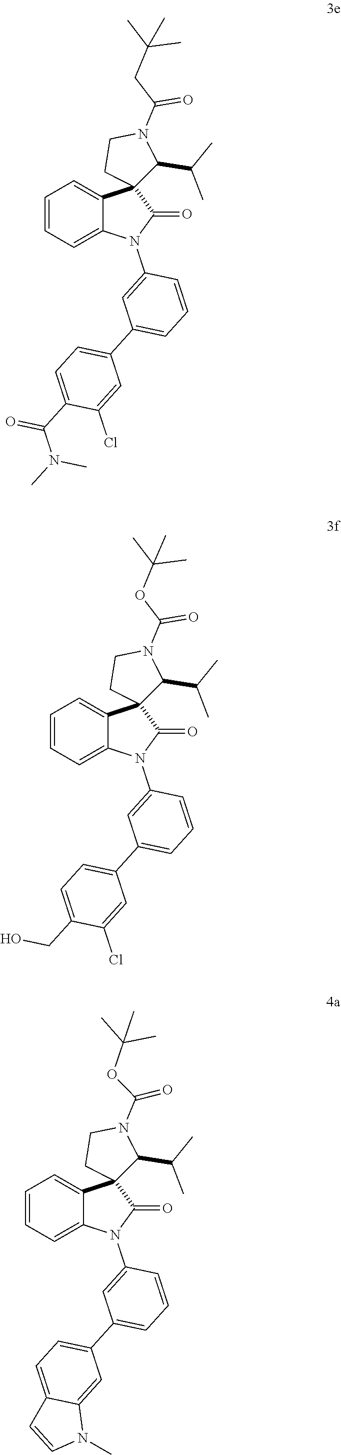

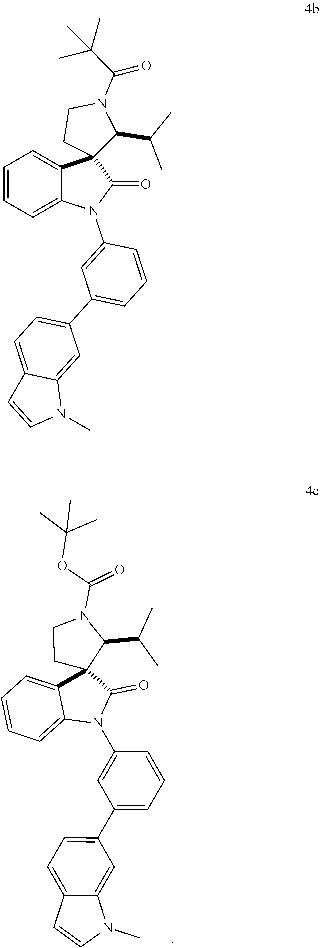

or a pharmaceutically acceptable salt thereof.

In another aspect, the compound is a sulfonamide-, sulfinamide- or sulfonimidamide-containing compound described in US20200115357A1 (see Formula I below).

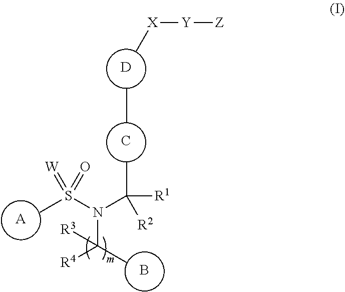

In another aspect, examples include a compound selected from the group:

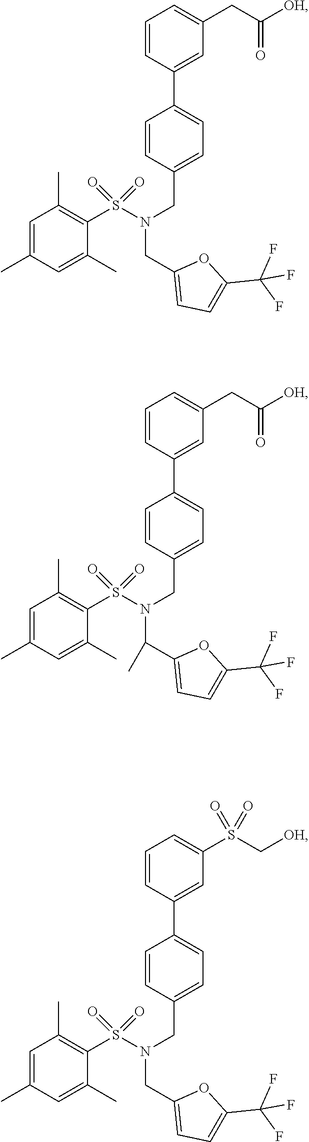 [SMILES: Cc1cc(C)c(S(=O)(=O)N(Cc2ccc(-c3cccc(CC(=O)O)c3)cc2)Cc2ccc(C(F)(F)F)o2)c(C)c1 ; Cc1cc(C)c(S(=O)(=O)N(Cc2ccc(-c3cccc(CC(=O)O)c3)cc2)C(C)c2ccc(C(F)(F)F)o2)c(C)c1 ; Cc1cc(C)c(S(=O)(=O)N(Cc2ccc(-c3cccc(S(=O)(=O)CO)c3)cc2)Cc2ccc(C(F)(F)F)o2)c(C)c1]

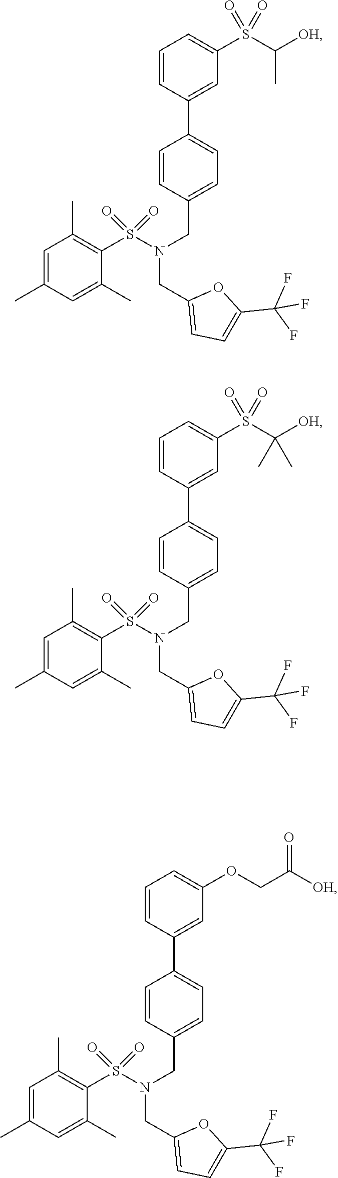 [SMILES: Cc1cc(C)c(S(=O)(=O)N(Cc2ccc(-c3cccc(S(=O)(=O)C(C)O)c3)cc2)Cc2ccc(C(F)(F)F)o2)c(C)c1 ; Cc1cc(C)c(S(=O)(=O)N(Cc2ccc(-c3cccc(S(=O)(=O)C(C)(C)O)c3)cc2)Cc2ccc(C(F)(F)F)o2)c(C)c1 ; Cc1cc(C)c(S(=O)(=O)N(Cc2ccc(-c3cccc(OCC(=O)O)c3)cc2)Cc2ccc(C(F)(F)F)o2)c(C)c1]

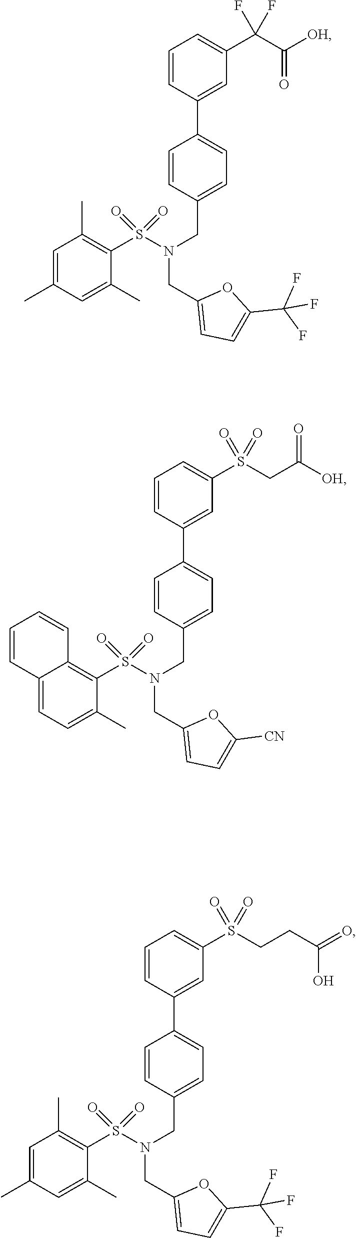 [SMILES: Cc1cc(C)c(S(=O)(=O)N(Cc2ccc(-c3cccc(C(F)(F)C(=O)O)c3)cc2)Cc2ccc(C(F)(F)F)o2)c(C)c1 ; Cc1ccc2ccccc2c1S(=O)(=O)N(Cc1ccc(-c2cccc(S(=O)(=O)CC(=O)O)c2)cc1)Cc1ccc(C#N)o1 ; Cc1cc(C)c(S(=O)(=O)N(Cc2ccc(-c3cccc(S(=O)(=O)CCC(=O)O)c3)cc2)Cc2ccc(C(F)(F)F)o2)c(C)c1]

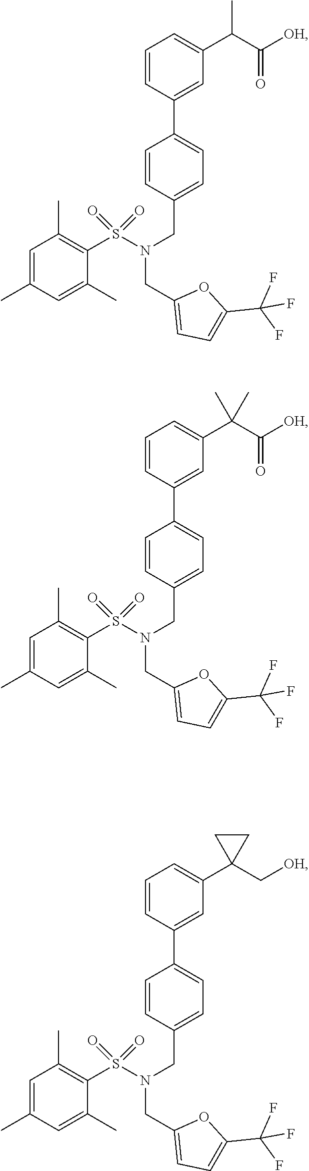 [SMILES: Cc1cc(C)c(S(=O)(=O)N(Cc2ccc(-c3cccc(C(C)C(=O)O)c3)cc2)Cc2ccc(C(F)(F)F)o2)c(C)c1 ; Cc1cc(C)c(S(=O)(=O)N(Cc2ccc(-c3cccc(C(C)(C)C(=O)O)c3)cc2)Cc2ccc(C(F)(F)F)o2)c(C)c1 ; Cc1cc(C)c(S(=O)(=O)N(Cc2ccc(-c3cccc(C4(CO)CC4)c3)cc2)Cc2ccc(C(F)(F)F)o2)c(C)c1]

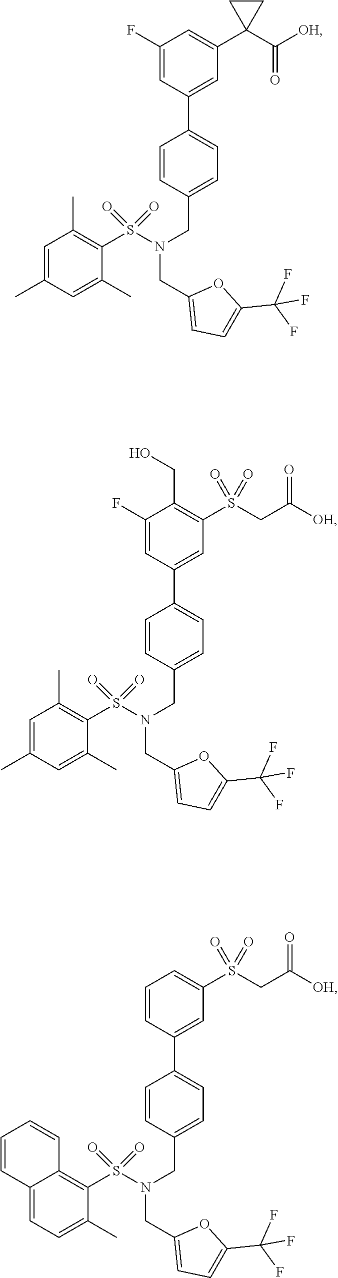 [SMILES: Cc1cc(C)c(S(=O)(=O)N(Cc2ccc(-c3cc(F)cc(C4(C(=O)O)CC4)c3)cc2)Cc2ccc(C(F)(F)F)o2)c(C)c1 ; Cc1cc(C)c(S(=O)(=O)N(Cc2ccc(-c3cc(F)c(CO)c(S(=O)(=O)CC(=O)O)c3)cc2)Cc2ccc(C(F)(F)F)o2)c(C)c1 ; Cc1ccc2ccccc2c1S(=O)(=O)N(Cc1ccc(-c2cccc(S(=O)(=O)CC(=O)O)c2)cc1)Cc1ccc(C(F)(F)F)o1]

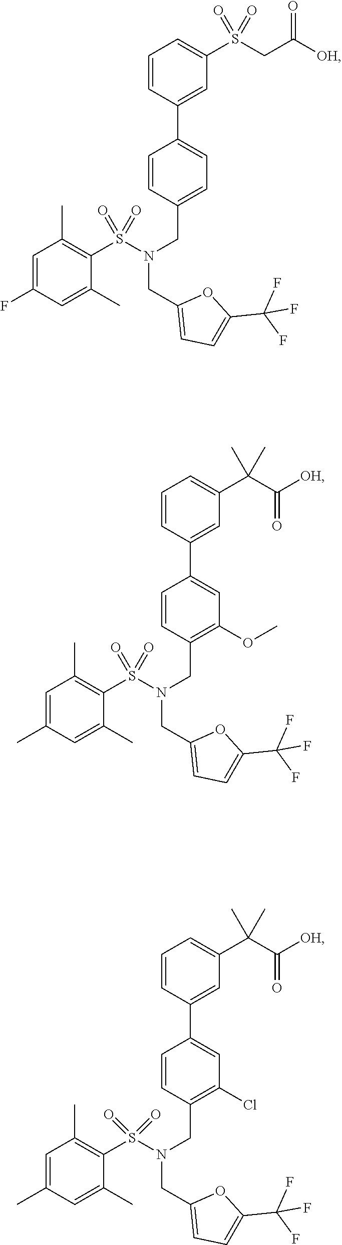 [SMILES: Cc1cc(F)cc(C)c1S(=O)(=O)N(Cc1ccc(-c2cccc(S(=O)(=O)CC(=O)O)c2)cc1)Cc1ccc(C(F)(F)F)o1 ; COc1cc(-c2cccc(C(C)(C)C(=O)O)c2)ccc1CN(Cc1ccc(C(F)(F)F)o1)S(=O)(=O)c1c(C)cc(C)cc1C ; Cc1cc(C)c(S(=O)(=O)N(Cc2ccc(C(F)(F)F)o2)Cc2ccc(-c3cccc(C(C)(C)C(=O)O)c3)cc2Cl)c(C)c1]

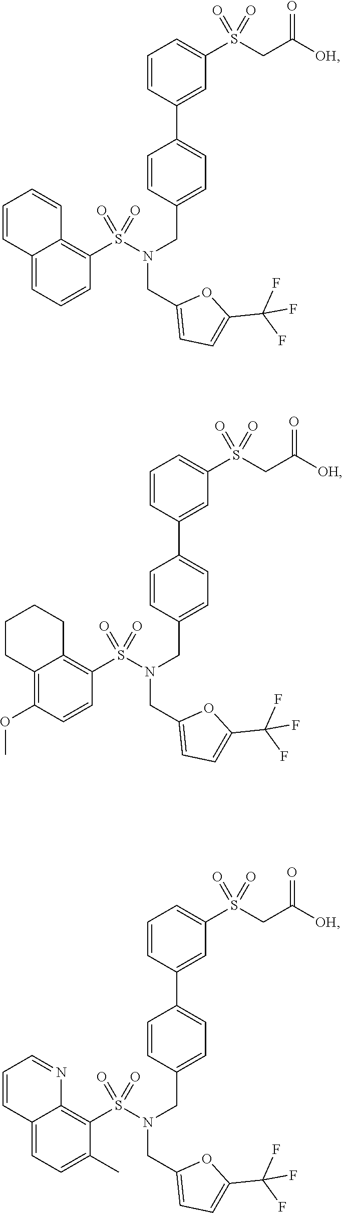 [SMILES: O=C(O)CS(=O)(=O)c1cccc(-c2ccc(CN(Cc3ccc(C(F)(F)F)o3)S(=O)(=O)c3cccc4ccccc34)cc2)c1 ; COc1ccc(S(=O)(=O)N(Cc2ccc(-c3cccc(S(=O)(=O)CC(=O)O)c3)cc2)Cc2ccc(C(F)(F)F)o2)c2c1CCCC2 ; Cc1ccc2cccnc2c1S(=O)(=O)N(Cc1ccc(-c2cccc(S(=O)(=O)CC(=O)O)c2)cc1)Cc1ccc(C(F)(F)F)o1]

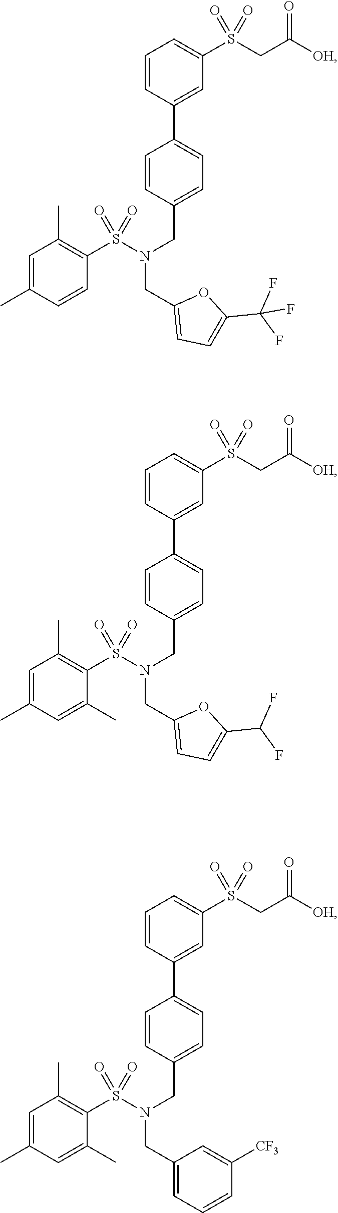 [SMILES: Cc1ccc(S(=O)(=O)N(Cc2ccc(-c3cccc(S(=O)(=O)CC(=O)O)c3)cc2)Cc2ccc(C(F)(F)F)o2)c(C)c1 ; Cc1cc(C)c(S(=O)(=O)N(Cc2ccc(-c3cccc(S(=O)(=O)CC(=O)O)c3)cc2)Cc2ccc(C(F)F)o2)c(C)c1 ; Cc1cc(C)c(S(=O)(=O)N(Cc2ccc(-c3cccc(S(=O)(=O)CC(=O)O)c3)cc2)Cc2cccc(C(F)(F)F)c2)c(C)c1]

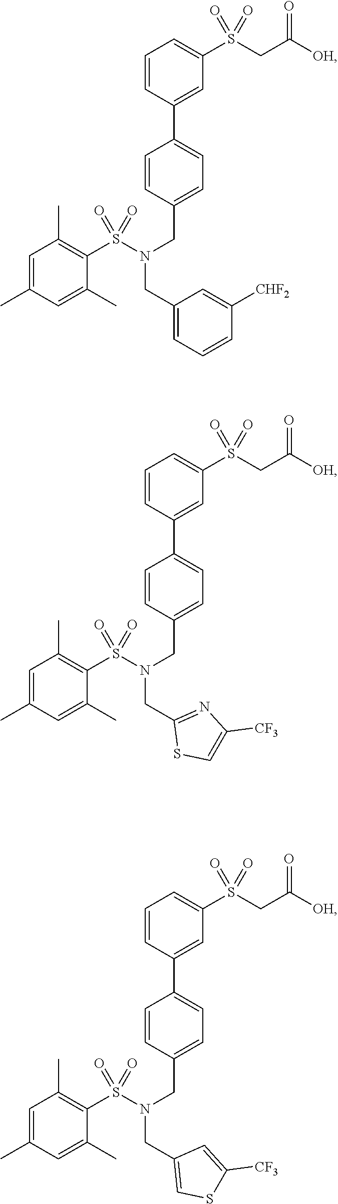 [SMILES: Cc1cc(C)c(S(=O)(=O)N(Cc2ccc(-c3cccc(S(=O)(=O)CC(=O)O)c3)cc2)Cc2cccc(C(F)F)c2)c(C)c1 ; Cc1cc(C)c(S(=O)(=O)N(Cc2ccc(-c3cccc(S(=O)(=O)CC(=O)O)c3)cc2)Cc2nc(C(F)(F)F)cs2)c(C)c1 ; Cc1cc(C)c(S(=O)(=O)N(Cc2ccc(-c3cccc(S(=O)(=O)CC(=O)O)c3)cc2)Cc2csc(C(F)(F)F)c2)c(C)c1]

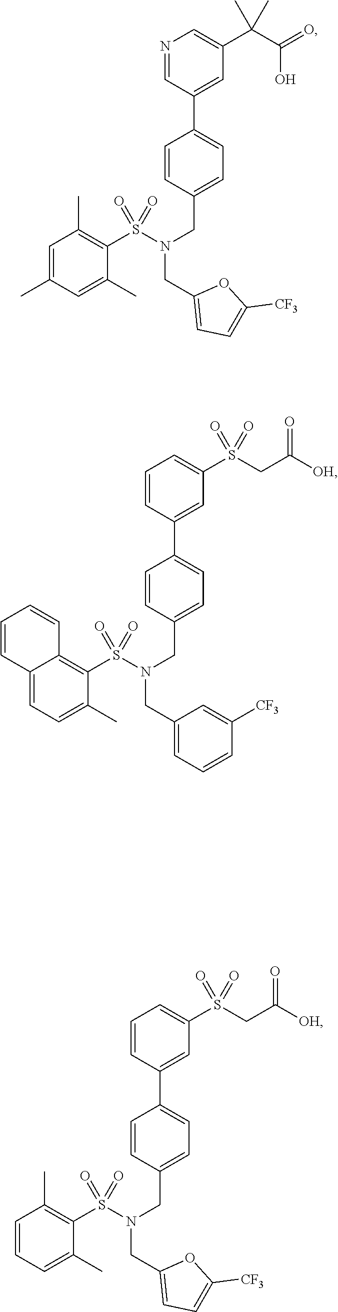 [SMILES: Cc1cc(C)c(S(=O)(=O)N(Cc2ccc(-c3cncc(C(C)(C)C(=O)O)c3)cc2)Cc2ccc(C(F)(F)F)o2)c(C)c1 ; Cc1ccc2ccccc2c1S(=O)(=O)N(Cc1ccc(-c2cccc(S(=O)(=O)CC(=O)O)c2)cc1)Cc1cccc(C(F)(F)F)c1 ; Cc1cccc(C)c1S(=O)(=O)N(Cc1ccc(-c2cccc(S(=O)(=O)CC(=O)O)c2)cc1)Cc1ccc(C(F)(F)F)o1]

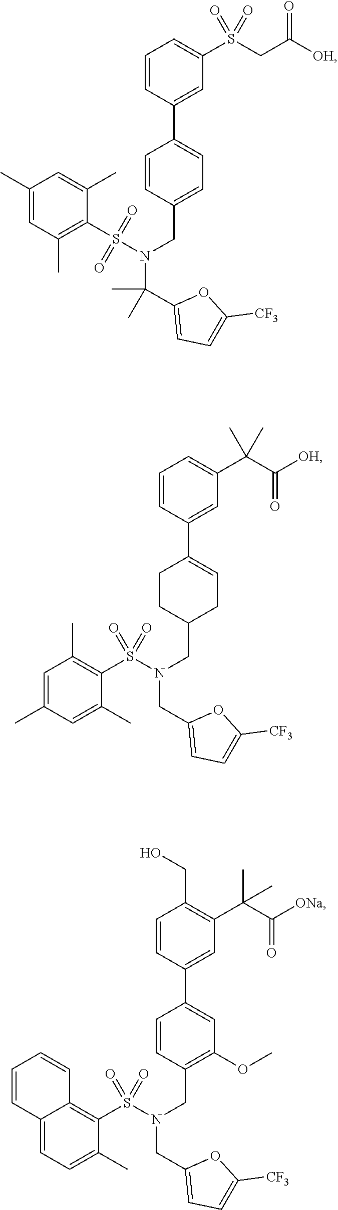

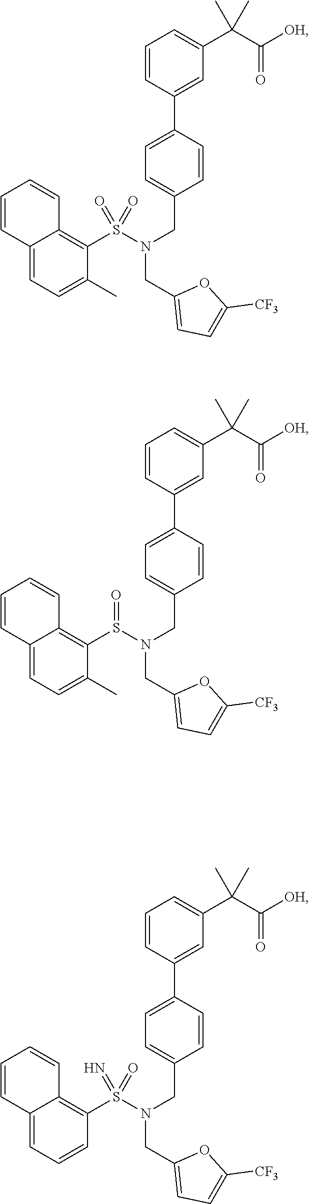 [SMILES: Cc1ccc2ccccc2c1S(=O)(=O)N(Cc1ccc(-c2cccc(C(C)(C)C(=O)O)c2)cc1)Cc1ccc(C(F)(F)F)o1 ; Cc1ccc2ccccc2c1S(=O)N(Cc1ccc(-c2cccc(C(C)(C)C(=O)O)c2)cc1)Cc1ccc(C(F)(F)F)o1 ; CC(C)(C(=O)O)c1cccc(-c2ccc(CN(Cc3ccc(C(F)(F)F)o3)S(=N)(=O)c3cccc4ccccc34)cc2)c1]

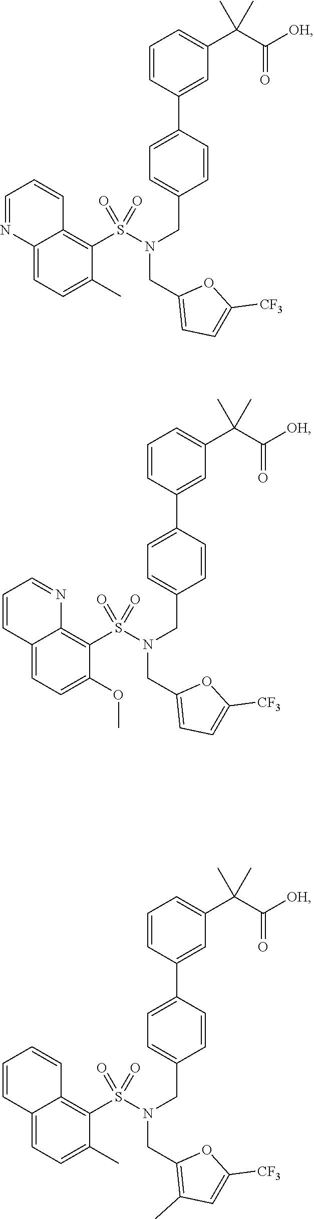 [SMILES: Cc1ccc2ncccc2c1S(=O)(=O)N(Cc1ccc(-c2cccc(C(C)(C)C(=O)O)c2)cc1)Cc1ccc(C(F)(F)F)o1 ; COc1ccc2cccnc2c1S(=O)(=O)N(Cc1ccc(-c2cccc(C(C)(C)C(=O)O)c2)cc1)Cc1ccc(C(F)(F)F)o1 ; Cc1cc(C(F)(F)F)oc1CN(Cc1ccc(-c2cccc(C(C)(C)C(=O)O)c2)cc1)S(=O)(=O)c1c(C)ccc2ccccc12]

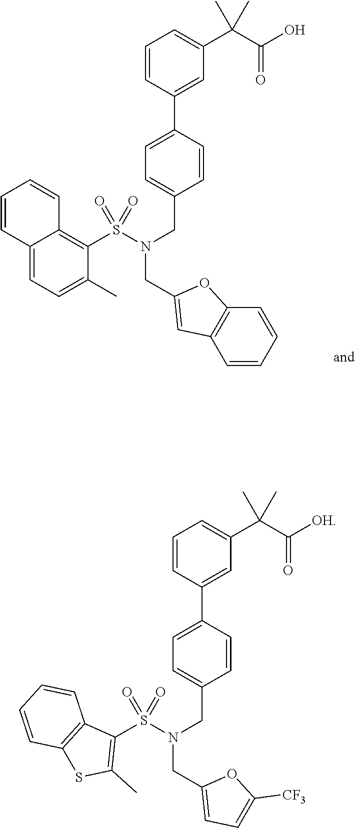 [SMILES: Cc1ccc2ccccc2c1S(=O)(=O)N(Cc1ccc(-c2cccc(C(C)(C)C(=O)O)c2)cc1)Cc1cc2ccccc2o1 ; Cc1sc2ccccc2c1S(=O)(=O)N(Cc1ccc(-c2cccc(C(C)(C)C(=O)O)c2)cc1)Cc1ccc(C(F)(F)F)o1]

In another aspect, the compound is a sulfonamide-containing compound described in US20210147398A (see Formula I below).

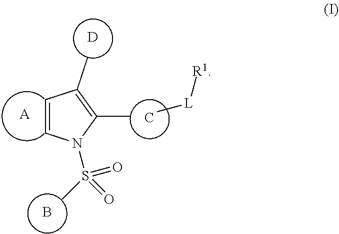

Specific examples include a compound selected from the group:

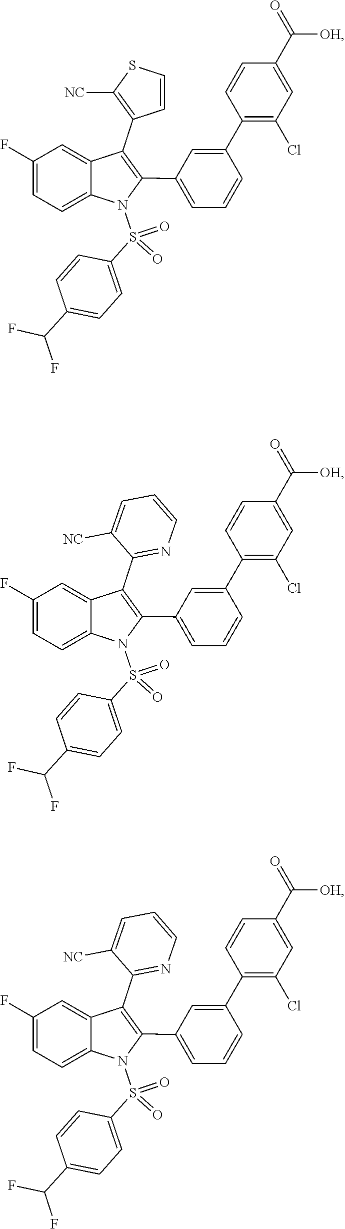 [SMILES: N#Cc1sccc1-c1c(-c2cccc(-c3ccc(C(=O)O)cc3Cl)c2)n(S(=O)(=O)c2ccc(C(F)F)cc2)c2ccc(F)cc12 ; N#Cc1cccnc1-c1c(-c2cccc(-c3ccc(C(=O)O)cc3Cl)c2)n(S(=O)(=O)c2ccc(C(F)F)cc2)c2ccc(F)cc12 ; N#Cc1cccnc1-c1c(-c2cccc(-c3ccc(C(=O)O)cc3Cl)c2)n(S(=O)(=O)c2ccc(C(F)F)cc2)c2ccc(F)cc12]

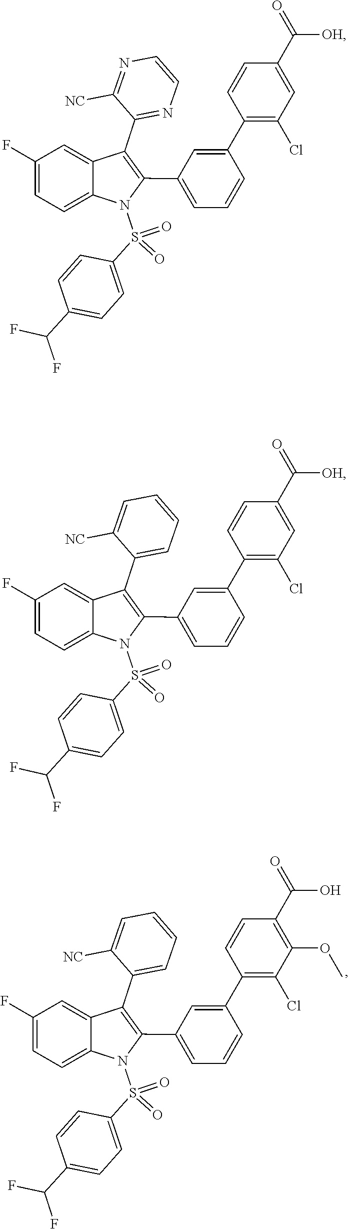 [SMILES: N#Cc1nccnc1-c1c(-c2cccc(-c3ccc(C(=O)O)cc3Cl)c2)n(S(=O)(=O)c2ccc(C(F)F)cc2)c2ccc(F)cc12 ; N#Cc1ccccc1-c1c(-c2cccc(-c3ccc(C(=O)O)cc3Cl)c2)n(S(=O)(=O)c2ccc(C(F)F)cc2)c2ccc(F)cc12 ; COc1c(C(=O)O)ccc(-c2cccc(-c3c(-c4ccccc4C#N)c4cc(F)ccc4n3S(=O)(=O)c3ccc(C(F)F)cc3)c2)c1Cl]

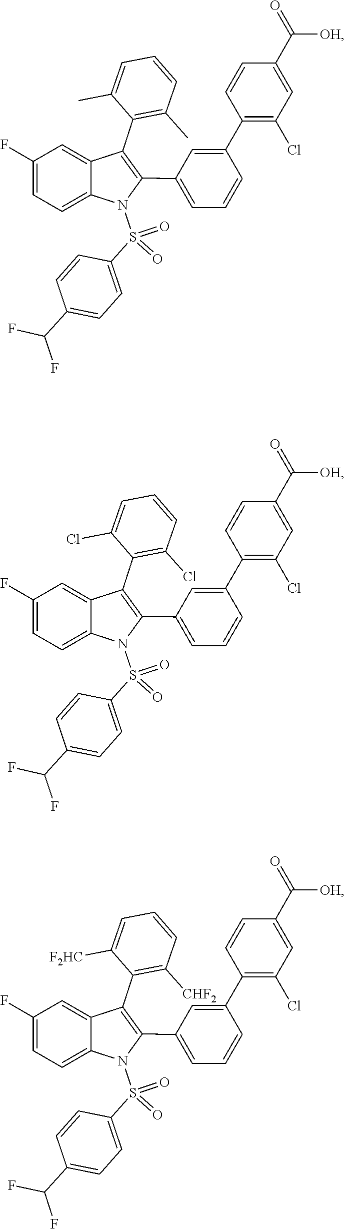 [SMILES: Cc1cccc(C)c1-c1c(-c2cccc(-c3ccc(C(=O)O)cc3Cl)c2)n(S(=O)(=O)c2ccc(C(F)F)cc2)c2ccc(F)cc12 ; O=C(O)c1ccc(-c2cccc(-c3c(-c4c(Cl)cccc4Cl)c4cc(F)ccc4n3S(=O)(=O)c3ccc(C(F)F)cc3)c2)c(Cl)c1 ; O=C(O)c1ccc(-c2cccc(-c3c(-c4c(C(F)F)cccc4C(F)F)c4cc(F)ccc4n3S(=O)(=O)c3ccc(C(F)F)cc3)c2)c(Cl)c1]

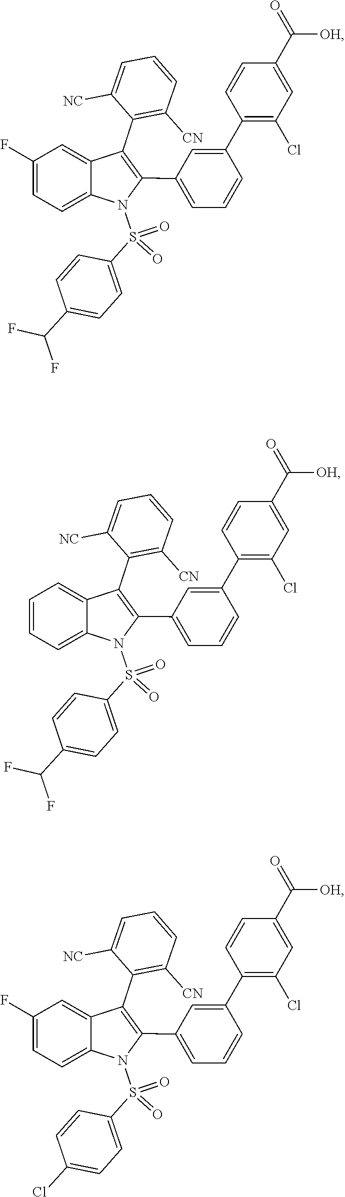 [SMILES: N#Cc1cccc(C#N)c1-c1c(-c2cccc(-c3ccc(C(=O)O)cc3Cl)c2)n(S(=O)(=O)c2ccc(C(F)F)cc2)c2ccc(F)cc12 ; N#Cc1cccc(C#N)c1-c1c(-c2cccc(-c3ccc(C(=O)O)cc3Cl)c2)n(S(=O)(=O)c2ccc(C(F)F)cc2)c2ccccc12 ; N#Cc1cccc(C#N)c1-c1c(-c2cccc(-c3ccc(C(=O)O)cc3Cl)c2)n(S(=O)(=O)c2ccc(Cl)cc2)c2ccc(F)cc12]

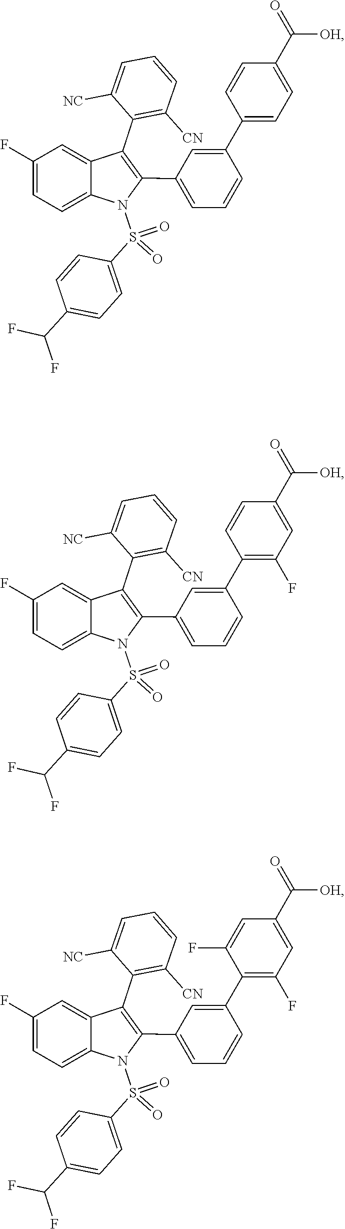 [SMILES: N#Cc1cccc(C#N)c1-c1c(-c2cccc(-c3ccc(C(=O)O)cc3)c2)n(S(=O)(=O)c2ccc(C(F)F)cc2)c2ccc(F)cc12 ; N#Cc1cccc(C#N)c1-c1c(-c2cccc(-c3ccc(C(=O)O)cc3F)c2)n(S(=O)(=O)c2ccc(C(F)F)cc2)c2ccc(F)cc12 ; N#Cc1cccc(C#N)c1-c1c(-c2cccc(-c3c(F)cc(C(=O)O)cc3F)c2)n(S(=O)(=O)c2ccc(C(F)F)cc2)c2ccc(F)cc12]

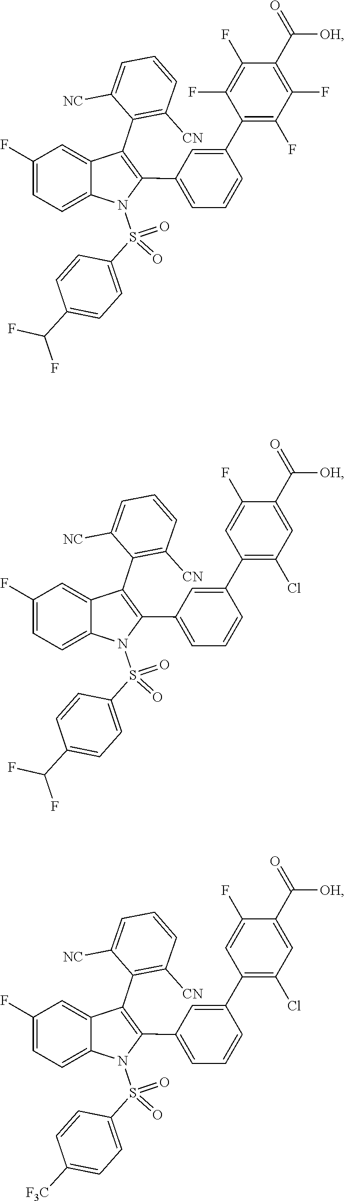 [SMILES: N#Cc1cccc(C#N)c1-c1c(-c2cccc(-c3c(F)c(F)c(C(=O)O)c(F)c3F)c2)n(S(=O)(=O)c2ccc(C(F)F)cc2)c2ccc(F)cc12 ; N#Cc1cccc(C#N)c1-c1c(-c2cccc(-c3cc(F)c(C(=O)O)cc3Cl)c2)n(S(=O)(=O)c2ccc(C(F)F)cc2)c2ccc(F)cc12 ; N#Cc1cccc(C#N)c1-c1c(-c2cccc(-c3cc(F)c(C(=O)O)cc3Cl)c2)n(S(=O)(=O)c2ccc(C(F)(F)F)cc2)c2ccc(F)cc12]

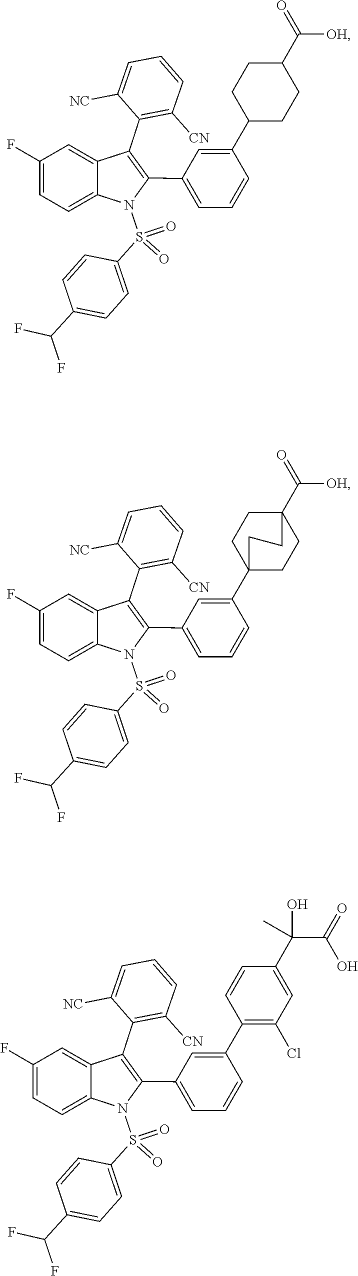 [SMILES: N#Cc1cccc(C#N)c1-c1c(-c2cccc(C3CCC(C(=O)O)CC3)c2)n(S(=O)(=O)c2ccc(C(F)F)cc2)c2ccc(F)cc12 ; N#Cc1cccc(C#N)c1-c1c(-c2cccc(C34CCC(C(=O)O)(CC3)CC4)c2)n(S(=O)(=O)c2ccc(C(F)F)cc2)c2ccc(F)cc12 ; CC(O)(C(=O)O)c1ccc(-c2cccc(-c3c(-c4c(C#N)cccc4C#N)c4cc(F)ccc4n3S(=O)(=O)c3ccc(C(F)F)cc3)c2)c(Cl)c1]

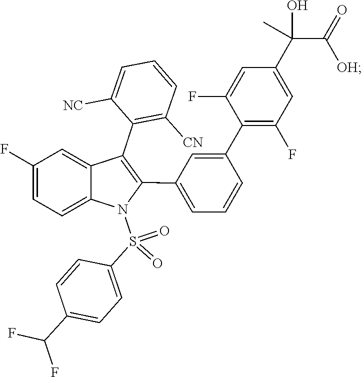 [SMILES: CC(O)(C(=O)O)c1cc(F)c(-c2cccc(-c3c(-c4c(C#N)cccc4C#N)c4cc(F)ccc4n3S(=O)(=O)c3ccc(C(F)F)cc3)c2)c(F)c1]

or pharmaceutically acceptable salt thereof.

In another aspect, the compound is: an amine or (thio)amide containing-containing compound described in US20200131144A1 (see Formula I below).

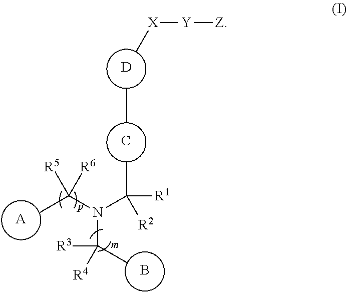

Specific examples include a compound selected from the group:

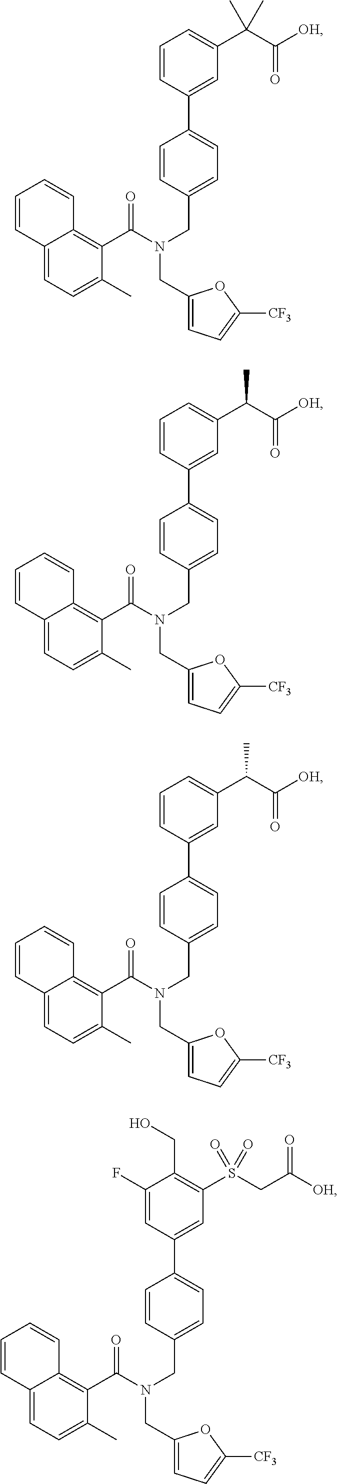

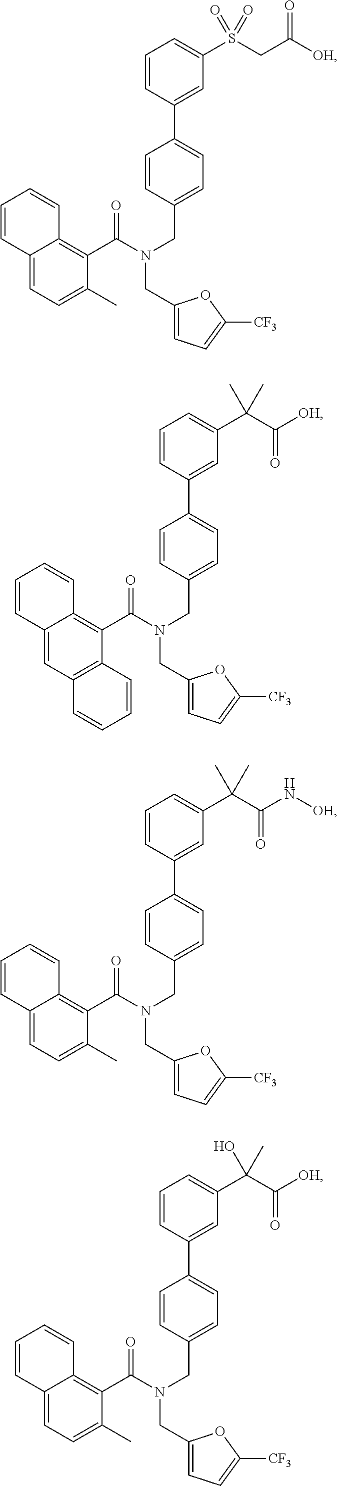

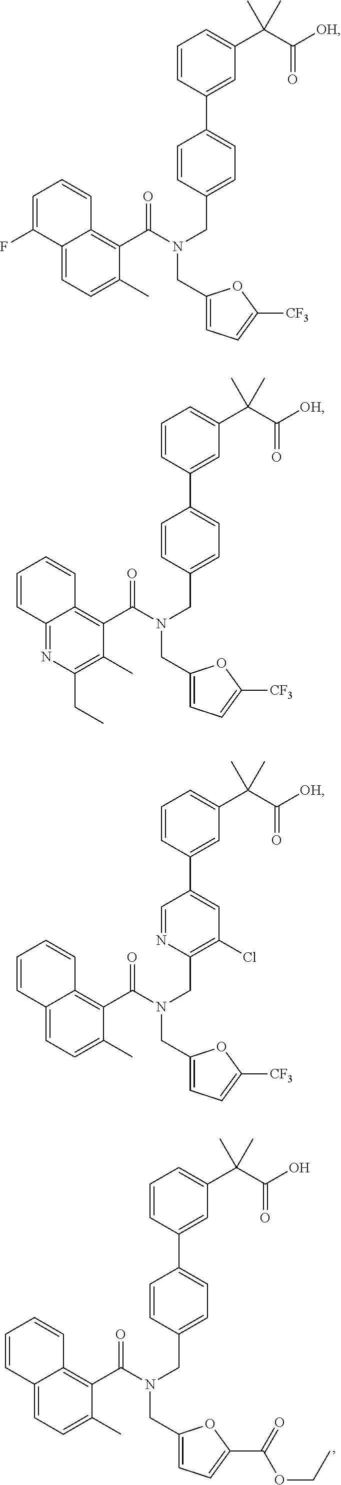

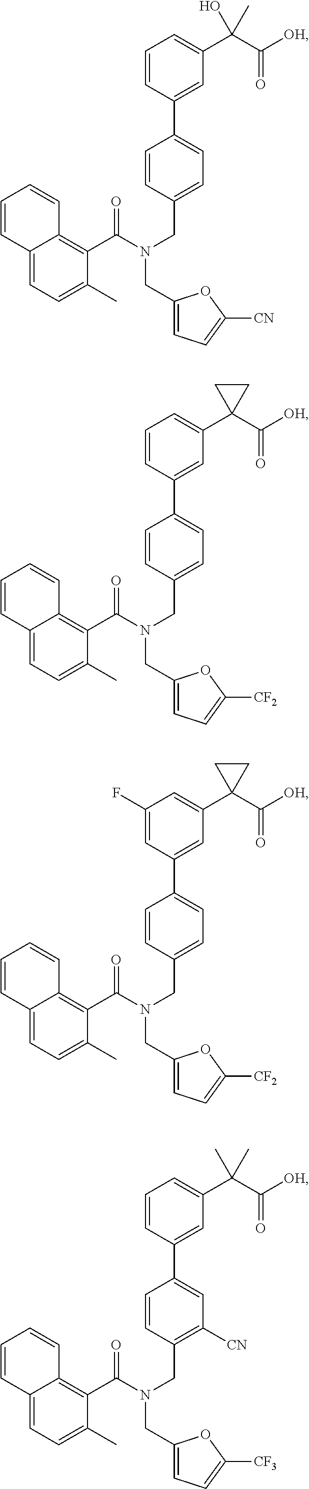

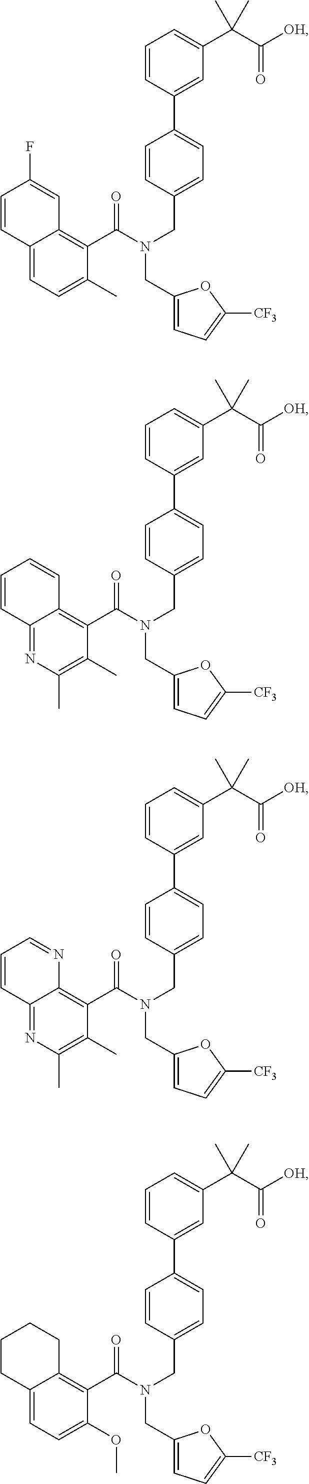

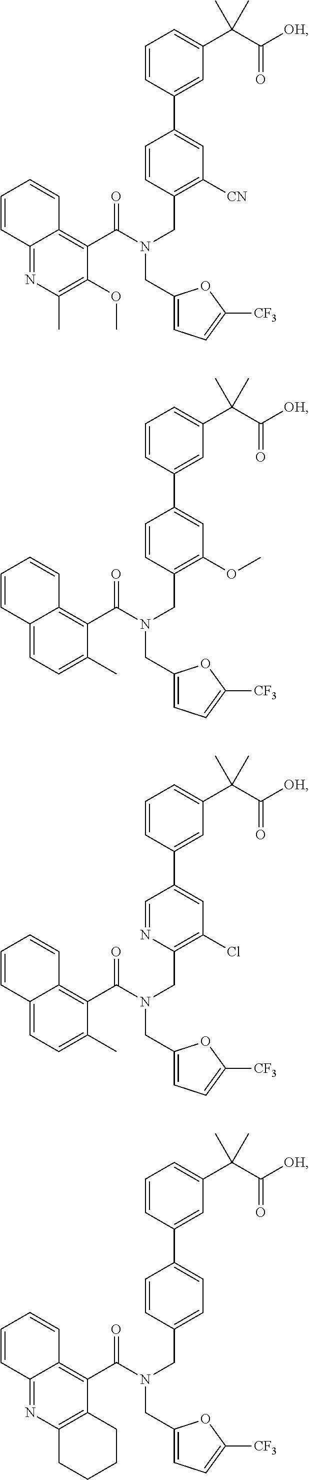

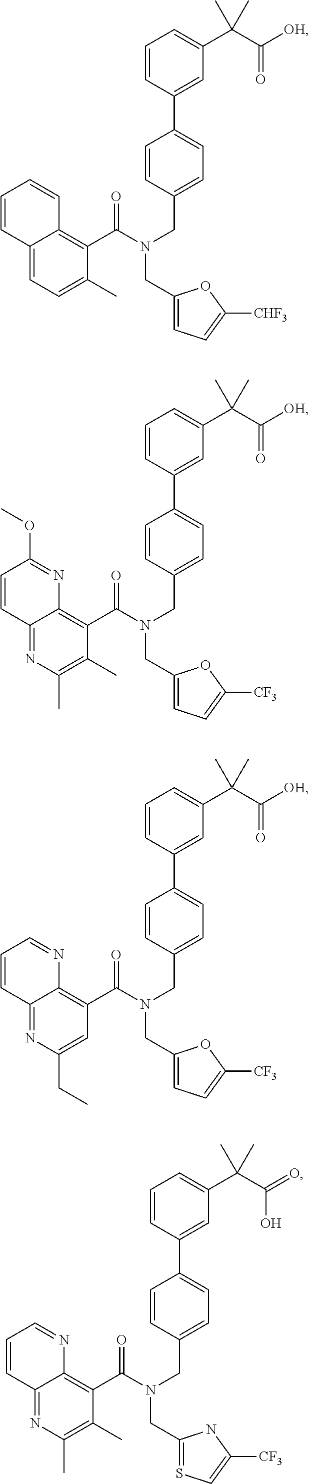

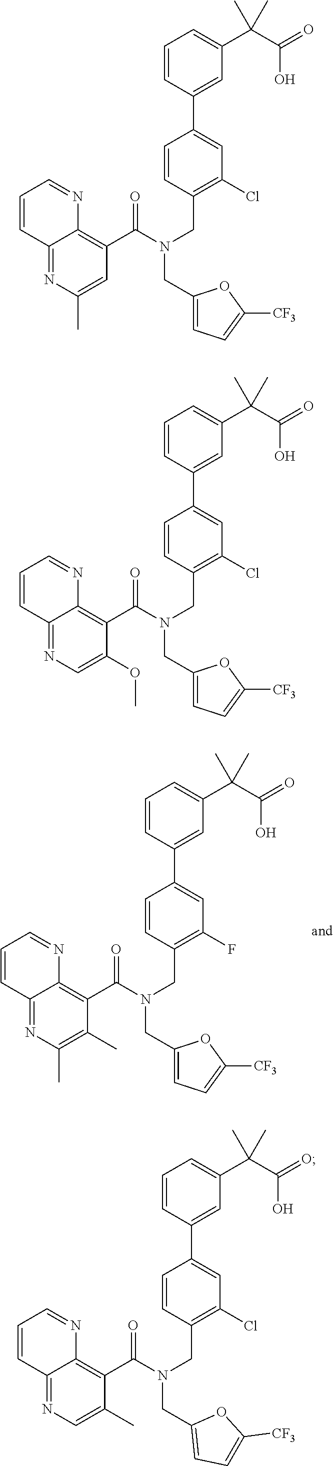

or pharmaceutically acceptable salt thereof.

In another aspect, the compound is: an amine-, carboxamide-, or thioamide-containing compound described in WO2020148325A1 (see Formula I below).

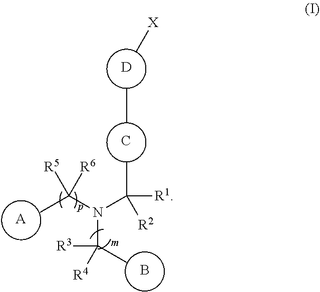

Specific examples include a compound selected from the group:

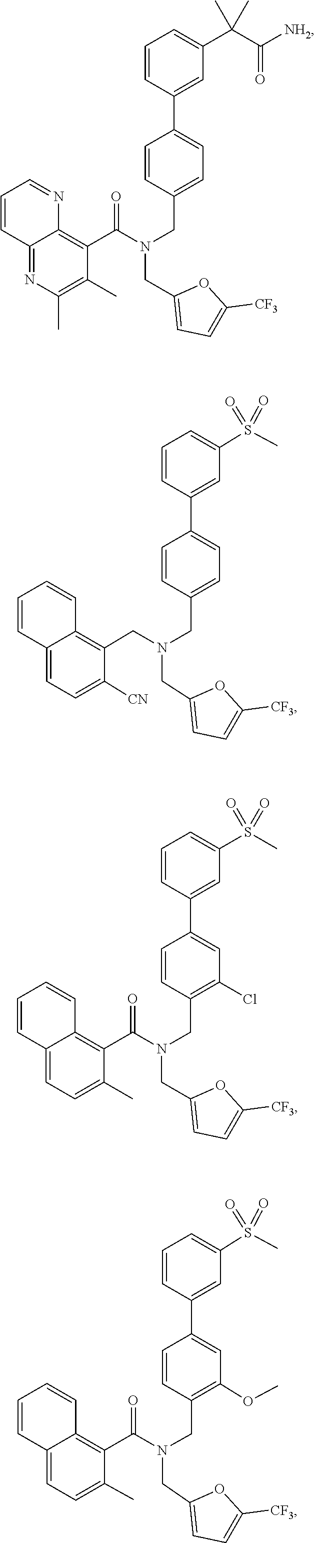 [SMILES: Cc1nc2cccnc2c(C(=O)N(Cc2ccc(-c3cccc(C(C)(C)C(N)=O)c3)cc2)Cc2ccc(C(F)(F)F)o2)c1C ; CS(=O)(=O)c1cccc(-c2ccc(CN(Cc3ccc(C(F)(F)F)o3)Cc3c(C#N)ccc4ccccc34)cc2)c1 ; Cc1ccc2ccccc2c1C(=O)N(Cc1ccc(C(F)(F)F)o1)Cc1ccc(-c2cccc(S(C)(=O)=O)c2)cc1Cl ; COc1cc(-c2cccc(S(C)(=O)=O)c2)ccc1CN(Cc1ccc(C(F)(F)F)o1)C(=O)c1c(C)ccc2ccccc12]

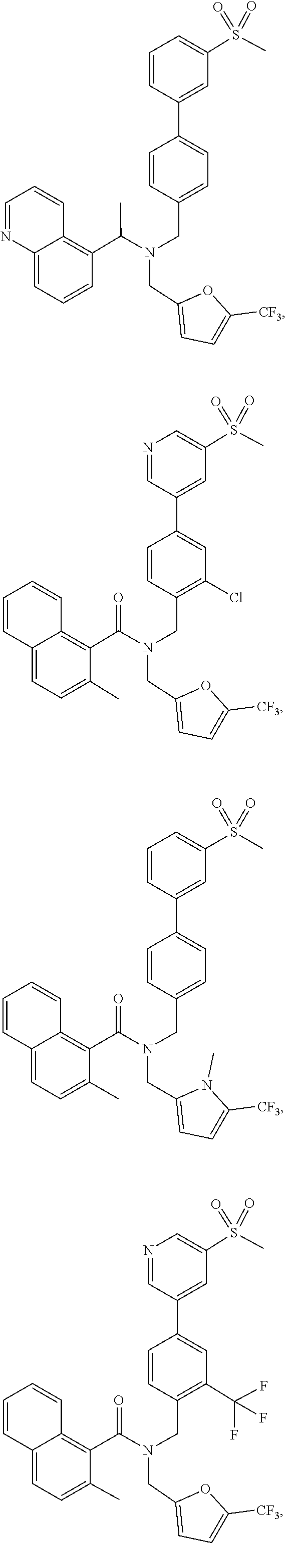 [SMILES: CC(c1cccc2ncccc12)N(Cc1ccc(-c2cccc(S(C)(=O)=O)c2)cc1)Cc1ccc(C(F)(F)F)o1 ; Cc1ccc2ccccc2c1C(=O)N(Cc1ccc(C(F)(F)F)o1)Cc1ccc(-c2cncc(S(C)(=O)=O)c2)cc1Cl ; Cc1ccc2ccccc2c1C(=O)N(Cc1ccc(-c2cccc(S(C)(=O)=O)c2)cc1)Cc1ccc(C(F)(F)F)n1C ; Cc1ccc2ccccc2c1C(=O)N(Cc1ccc(C(F)(F)F)o1)Cc1ccc(-c2cncc(S(C)(=O)=O)c2)cc1C(F)(F)F]

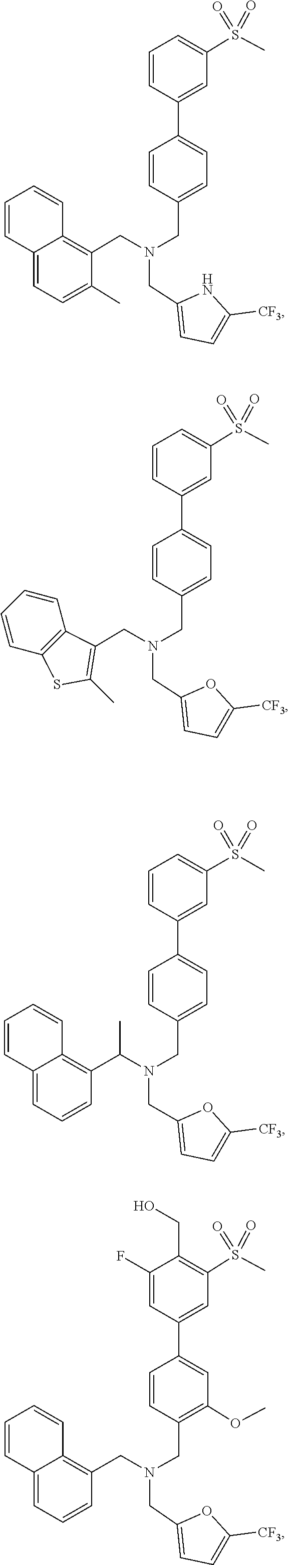 [SMILES: Cc1ccc2ccccc2c1CN(Cc1ccc(-c2cccc(S(C)(=O)=O)c2)cc1)Cc1ccc(C(F)(F)F)[nH]1 ; Cc1sc2ccccc2c1CN(Cc1ccc(-c2cccc(S(C)(=O)=O)c2)cc1)Cc1ccc(C(F)(F)F)o1 ; CC(c1cccc2ccccc12)N(Cc1ccc(-c2cccc(S(C)(=O)=O)c2)cc1)Cc1ccc(C(F)(F)F)o1 ; COc1cc(-c2cc(F)c(CO)c(S(C)(=O)=O)c2)ccc1CN(Cc1ccc(C(F)(F)F)o1)Cc1cccc2ccccc12]

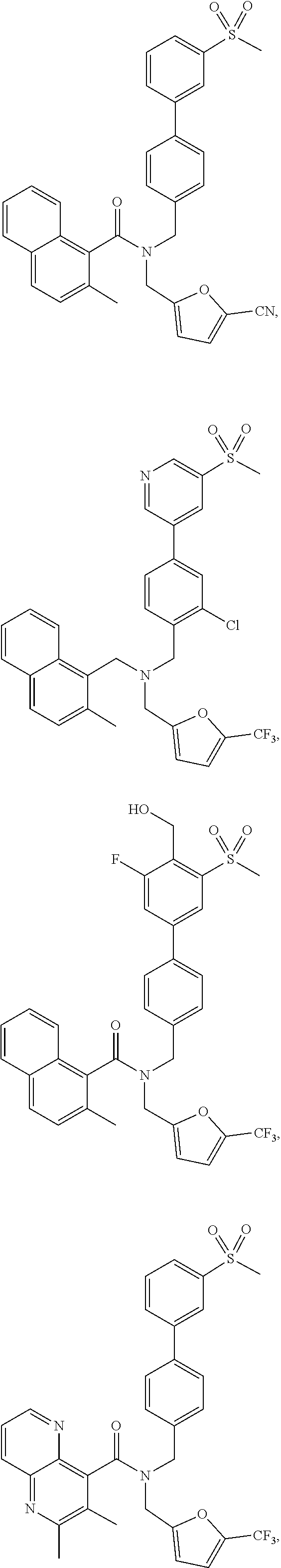 [SMILES: Cc1ccc2ccccc2c1C(=O)N(Cc1ccc(-c2cccc(S(C)(=O)=O)c2)cc1)Cc1ccc(C#N)o1 ; Cc1ccc2ccccc2c1CN(Cc1ccc(C(F)(F)F)o1)Cc1ccc(-c2cncc(S(C)(=O)=O)c2)cc1Cl ; Cc1ccc2ccccc2c1C(=O)N(Cc1ccc(-c2cc(F)c(CO)c(S(C)(=O)=O)c2)cc1)Cc1ccc(C(F)(F)F)o1 ; Cc1nc2cccnc2c(C(=O)N(Cc2ccc(-c3cccc(S(C)(=O)=O)c3)cc2)Cc2ccc(C(F)(F)F)o2)c1C]

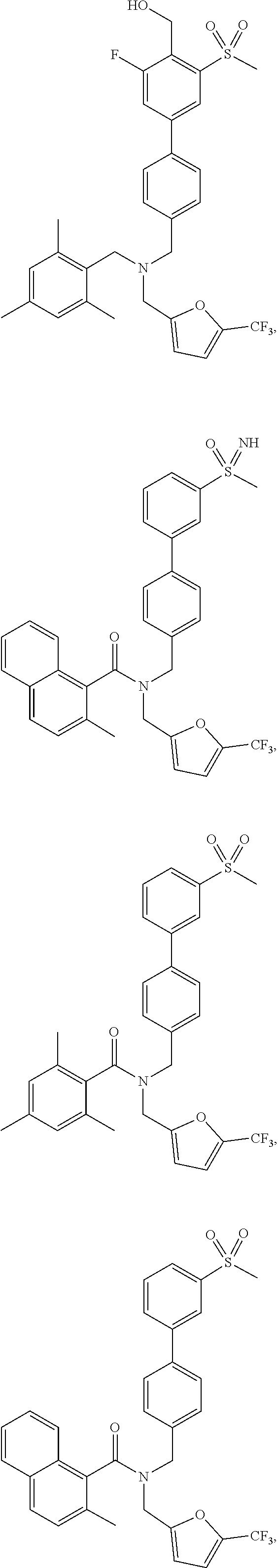

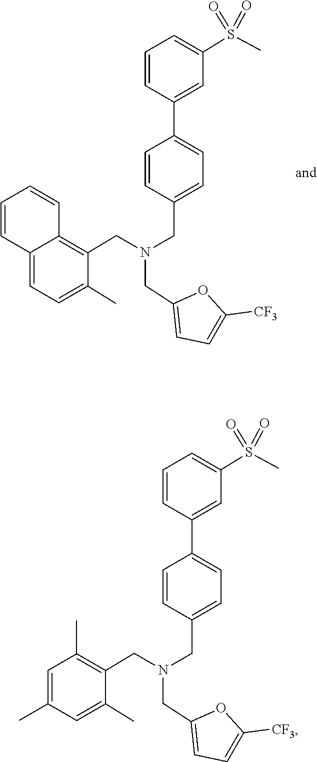

or a pharmaceutically acceptable salt thereof.

### LXR Antagonists

In another aspect, the compound is:

GSK2033

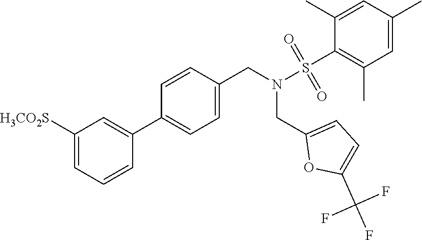

or a pharmaceutically acceptable salt thereof.

The invention may be embodied in other specific forms without departing from the spirit or essential attributes thereof. This invention encompasses all combinations of aspects of the invention noted herein. It is understood that any and all embodiments of the invention may be taken in conjunction with any other embodiment or embodiments to describe additional embodiments. It is also to be understood that each individual element of the embodiments is intended to be taken individually as its own independent embodiment. Furthermore, any element of an embodiment is meant to be combined with any and all other elements from any embodiment to describe an additional embodiment.

### Formulation and Dosage

Formulation of pharmaceutical compositions in tablets, capsules, and topical gels, topical creams or suppositories is well known in the art and is described, for example, in United States Patent Application Publication No. 2004/0023290 by Griffin et al. Formulation of pharmaceutical compositions as patches such as transdermal patches is well known in the art and is described, for example, in U.S. Pat. No. 7,728,042 to Eros et al. Lyophilized dosage fills are also well known in the art.

The use of prodrug systems is described in T. Jarvinen et al., “Design and Pharmaceutical Applications of Prodrugs” in Drug Discovery Handbook (S. C. Gad, ed., Wiley-Interscience, Hoboken, N.J., 2005), ch. 17, pp. 733-796; U.S. Pat. No. 7,879,896 to Allegretto et al.’ S. Prasad et al., “Delivering Multiple Anticancer Peptides as a Single Prodrug Using Lysyl-Lysine as a Facile Linker,” J. Peptide Sci. 13: 458-467 (2007); U.S. Pat. No. 7,619,005 to Epstein et al.; U.S. Pat. No. 6,443,898 to Unger et al.

Prodrugs and active metabolites of a compound may be identified using routine techniques known in the art. See, e.g., Bertolini et al., J. Med. Chem., 40, 2011-2016 (1997); Shan et al., J. Pharm. Sci., 86 (7), 765-767; Bagshawe, Drug Dev. Res., 34, 220-230 (1995); Bodor, Advances in Drug Res., 13, 224-331 (1984); Bundgaard, Design of Prodrugs (Elsevier Press 1985); Larsen, Design and Application of Prodrugs, Drug Design and Development (Krogsgaard-Larsen et al., eds., Harwood Academic Publishers, 1991); Dear et al., J. Chromatogr. B, 748, 281-293 (2000); Spraul et al., J. Pharmaceutical & Biomedical Analysis, 10, 601-605 (1992); and Prox et al., Xenobiol., 3, 103-112 (1992).

When the pharmacologically active compound in a pharmaceutical composition possesses a sufficiently acidic, a sufficiently basic, or both a sufficiently acidic and a sufficiently basic functional group, these group or groups can accordingly react with any of several inorganic or organic bases, and inorganic and organic acids, to form a pharmaceutically acceptable salt. Exemplary pharmaceutically acceptable salts include those salts prepared by reaction of the pharmacologically active compound with a mineral or organic acid or an inorganic base, such as salts including sulfates, pyrosulfates, bisulfates, sulfites, bisulfites, phosphates, monohydrogenphosphates, dihydrogenphosphates, metaphosphates, pyrophosphates, chlorides, bromides, iodides, acetates, propionates, decanoates, caprylates, acrylates, formates, isobutyrates, caproates, heptanoates, propiolates, oxalates, malonates, succinates, suberates, sebacates, fumarates, maleates, butyne-1,4-dioates, hexyne-1,6-dioates, benzoates, chlorobenzoates, methylbenzoates, dinitrobenzoates, hydroxybenzoates, methoxybenzoates, phthalates, sulfonates, xylenesulfonates, phenylacetates, phenylpropionates, phenylbutyrates, citrates, lactates, .beta.-hydroxybutyrates, glycolates, tartrates, methane-sulfonates, propanesulfonates, naphthalene-1-sulfonates, naphthalene-2-sulfonates, and mandelates.

If the pharmacologically active compound has one or more basic functional groups, the desired pharmaceutically acceptable salt may be prepared by any suitable method available in the art, for example, treatment of the free base with an inorganic acid, such as hydrochloric acid, hydrobromic acid, sulfuric acid, nitric acid, phosphoric acid and the like, or with an organic acid, such as acetic acid, maleic acid, succinic acid, mandelic acid, fumaric acid, malonic acid, pyruvic acid, oxalic acid, glycolic acid, salicylic acid, a pyranosidyl acid, such as glucuronic acid or galacturonic acid, an alpha-hydroxy acid, such as citric acid or tartaric acid, an amino acid, such as aspartic acid or glutamic acid, an aromatic acid, such as benzoic acid or cinnamic acid, a sulfonic acid, such as p-toluenesulfonic acid or ethanesulfonic acid, or the like.

If the pharmacologically active compound has one or more acidic functional groups, the desired pharmaceutically acceptable salt may be prepared by any suitable method available in the art, for example, treatment of the free acid with an inorganic or organic base, such as an amine (primary, secondary or tertiary), an alkali metal hydroxide or alkaline earth metal hydroxide, or the like. Illustrative examples of suitable salts include organic salts derived from amino acids, such as glycine and arginine, ammonia, primary, secondary, and tertiary amines, and cyclic amines, such as piperidine, morpholine and piperazine, and inorganic salts derived from sodium, calcium, potassium, magnesium, manganese, iron, copper, zinc, aluminum and lithium.

In the case of agents that are solids, it is understood by those skilled in the art that the compounds and salts may exist in different crystal or polymorphic forms, all of which are intended to be within the scope of the invention and specified formulas.

The amount of a given pharmacologically active agent in a unit dose of a pharmaceutical composition will vary depending upon factors such as the particular compound, disease condition and its severity, the identity (e.g., weight) of the subject in need of treatment, but can nevertheless be routinely determined by one skilled in the art. Typically, such pharmaceutical compositions include a therapeutically effective quantity of the pharmacologically active agent and an inert pharmaceutically acceptable carrier or diluent. Typically, these compositions are prepared in unit dosage form appropriate for the chosen route of administration, such as oral administration or parenteral administration. A pharmacologically active agent as described above can be administered in conventional dosage form prepared by combining a therapeutically effective amount of such a pharmacologically active agent as an active ingredient with appropriate pharmaceutical carriers or diluents according to conventional procedures. These procedures may involve mixing, granulating, and compressing or dissolving the ingredients as appropriate to the desired preparation. The pharmaceutical carrier employed may be either a solid or liquid. Exemplary of solid carriers are lactose, sucrose, talc, gelatin, agar, pectin, acacia, magnesium stearate, stearic acid and the like. Exemplary of liquid carriers are syrup, peanut oil, olive oil, water and the like. Similarly, the carrier or diluent may include time-delay or time-release material known in the art, such as glyceryl monostearate or glyceryl distearate alone or with a wax, ethylcellulose, hydroxypropylmethylcellulose, methylmethacrylate and the like.

A variety of pharmaceutical forms can be employed. Thus, if a solid carrier is used, the preparation can be tableted, placed in a hard gelatin capsule in powder or pellet form or in the form of a troche or lozenge. The amount of solid carrier may vary, but generally will be from about 25 mg to about 1 g. If a liquid carrier is used, the preparation will be in the form of syrup, emulsion, soft gelatin capsule, sterile injectable solution or suspension in an ampoule or vial or non-aqueous liquid suspension.

To obtain a stable water-soluble dose form, a pharmaceutically acceptable salt of a pharmacologically active agent as described above is dissolved in an aqueous solution of an organic or inorganic acid, such as 0.3 M solution of succinic acid or citric acid. If a soluble salt form is not available, the agent may be dissolved in a suitable cosolvent or combinations of cosolvents. Examples of suitable cosolvents include, but are not limited to, alcohol, propylene glycol, polyethylene glycol 300, polysorbate 80, glycerin and the like in concentrations ranging from 0-60% of the total volume. In an exemplary embodiment, a compound of Formula I is dissolved in DMSO and diluted with water. The composition may also be in the form of a solution of a salt form of the active ingredient in an appropriate aqueous vehicle such as water or isotonic saline or dextrose solution.

It will be appreciated that the actual dosages of the agents used in the compositions of this invention will vary according to the complex being used, the composition formulated, the mode of administration and the site, host, disease, and/or condition being treated. Actual dosage levels of the active ingredients in the pharmaceutical compositions of the invention can be varied to obtain an amount of the active ingredient which is effective to achieve the desired therapeutic response for a particular subject, composition, and mode of administration, without being toxic to the subject. The selected dosage level depends upon a variety of pharmacokinetic factors including the activity of the therapeutic agent, the route of administration, the time of administration, the rate of excretion of the compound being employed, the severity of the condition, other health considerations affecting the subject, and the status of liver and kidney function of the subject. It also depends on the duration of the treatment, other drugs, compounds and/or materials used in combination with the therapeutic agent employed, as well as the age, weight, condition, general health and prior medical history of the subject being treated, and like factors. Methods for determining optimal dosages are described in the art, e.g., Remington: The Science and Practice of Pharmacy, Mack Publishing Co., 20.sup.th ed., 2000. Optimal dosages for a given set of conditions can be ascertained by those skilled in the art using conventional dosage-determination tests in view of the experimental data for an agent.

For oral administration, an exemplary daily dose generally employed is from about 0.001 to about 3000 mg/kg of body weight, with courses of treatment repeated at appropriate intervals. In some embodiments, the daily dose is from about 1 to 3000 mg/kg of body weight. Typical daily doses in a patient may be anywhere between about 500 mg to about 3000 mg, given once or twice daily, e.g., 3000 mg can be given twice daily for a total dose of 6000 mg. In another aspect, the dose is between about 1000 to about 3000 mg. In another aspect, the dose is between about 1500 to about 2800 mg. In other embodiments, the dose is between about 2000 to about 3000 mg. Typically, doses are from about 1 mg/m² to about 40 mg/m². Further examples include doses that are from about 5 mg/m² to about 25 mg/m².

Plasma concentrations in the subjects may be between about 100 μM to about 1000 μM. In some embodiments, the plasma concentration may be between about 200 μM to about 800 μM. In other embodiments, the concentration is about 300 μM to about 600 μM. In still other embodiments the plasma concentration may be between about 400 μM to about 800 μM. In another alternative, the plasma concentration can be between about 0.5 μM to about 20 μM, typically 1 μM to about 10 μM. Administration of prodrugs is typically dosed at weight levels, which are chemically equivalent to the weight levels of the fully active form.

The compositions of the invention may be manufactured using techniques generally known for preparing pharmaceutical compositions, e.g., by conventional techniques such as mixing, dissolving, granulating, dragee-making, levitating, emulsifying, encapsulating, entrapping or lyophilizing. Pharmaceutical compositions may be formulated in a conventional manner using one or more physiologically acceptable carriers, which may be selected from excipients and auxiliaries that facilitate processing of the active compounds into preparations, which can be used pharmaceutically.

Proper formulation is dependent upon the route of administration chosen. For injection, the agents of the invention may be formulated into aqueous solutions, e.g., in physiologically compatible buffers such as Hanks's solution, Ringer's solution, or physiological saline buffer. For transmucosal administration, penetrants appropriate to the barrier to be permeated are used in the formulation. Such penetrants are generally known in the art.

For oral administration, the compounds can be formulated readily by combining the active compounds with pharmaceutically acceptable carriers known in the art. Such carriers enable the compounds of the invention to be formulated as tablets, pills, dragees, capsules, liquids, gels, syrups, slurries, solutions, suspensions and the like, for oral ingestion by a patient to be treated. Pharmaceutical preparations for oral use can be obtained using a solid excipient in admixture with the active ingredient (agent), optionally grinding the resulting mixture, and processing the mixture of granules after adding suitable auxiliaries, if desired, to obtain tablets or dragee cores. Suitable excipients include: fillers such as sugars, including lactose, sucrose, mannitol, or sorbitol; and cellulose preparations, for example, maize starch, wheat starch, rice starch, potato starch, gelatin, gum, methyl cellulose, hydroxypropylmethyl-cellulose, sodium carboxymethylcellulose, or polyvinylpyrrolidone (PVP). If desired, disintegrating agents may be added, such as crosslinked polyvinyl pyrrolidone, agar, or alginic acid or a salt thereof such as sodium alginate.

Dragee cores are provided with suitable coatings. For this purpose, concentrated sugar solutions may be used, which may optionally contain gum arabic, polyvinyl pyrrolidone, Carbopol gel, polyethylene glycol, and/or titanium dioxide, lacquer solutions, and suitable organic solvents or solvent mixtures. Dyestuffs or pigments may be added to the tablets or dragee coatings for identification or to characterize different combinations of active agents.

Pharmaceutical preparations which can be used orally include push-fit capsules made of gelatin, as well as soft, sealed capsules made of gelatin and a plasticizer, such as glycerol or sorbitol. The push-fit capsules can contain the active ingredients in admixture with fillers such as lactose, binders such as starches, and/or lubricants such as talc or magnesium stearate, and, optionally, stabilizers. In soft capsules, the active agents may be dissolved or suspended in suitable liquids, such as fatty oils, liquid paraffin, or liquid polyethylene glycols. In addition, stabilizers may be added. All formulations for oral administration should be in dosages suitable for such administration. For buccal administration, the compositions may take the form of tablets or lozenges formulated in conventional manner.

Pharmaceutical formulations for parenteral administration can include aqueous solutions or suspensions. Suitable lipophilic solvents or vehicles include fatty oils such as sesame oil or synthetic fatty acid esters, such as ethyl oleate or triglycerides. Aqueous injection suspensions may contain substances which increase the viscosity of the suspension, such as sodium carboxymethyl cellulose, sorbitol, or dextran. Optionally, the suspension may also contain suitable stabilizers or modulators which increase the solubility or dispersibility of the composition to allow for the preparation of highly concentrated solutions or can contain suspending or dispersing agents. Pharmaceutical preparations for oral use can be obtained by combining the pharmacologically active agent with solid excipients, optionally grinding a resulting mixture, and processing the mixture of granules, after adding suitable auxiliaries, if desired, to obtain tablets or dragee cores. Suitable excipients are, in particular, fillers such as sugars, including lactose, sucrose, mannitol, or sorbitol; cellulose preparations such as, for example, maize starch, wheat starch, rice starch, potato starch, gelatin, gum tragacanth, methyl cellulose, hydroxypropylmethyl-cellulose, sodium carboxymethylcellulose, and/or polyvinylpyrrolidone (PVP). If desired, disintegrating modulators may be added, such as the cross-linked polyvinyl pyrrolidone, agar, or alginic acid or a salt thereof such as sodium alginate.

Other ingredients such as stabilizers, for example, antioxidants such as sodium citrate, ascorbyl palmitate, propyl gallate, reducing agents, ascorbic acid, vitamin E, sodium bisulfite, butylated hydroxytoluene, BHA, acetylcysteine, monothioglycerol, phenyl-.alpha.-naphthylamine, or lecithin can be used. Also, chelators such as EDTA can be used.

Other ingredients that are conventional in pharmaceutical compositions and formulations, such as lubricants in tablets or pills, coloring agents, or flavoring agents, can be used. Also, conventional pharmaceutical excipients or carriers can be used. The pharmaceutical excipients can include, but are not necessarily limited to, calcium carbonate, calcium phosphate, various sugars or types of starch, cellulose derivatives, gelatin, vegetable oils, polyethylene glycols and physiologically compatible solvents. Other pharmaceutical excipients are well known in the art. Exemplary pharmaceutically acceptable carriers include, but are not limited to, any and/or all of solvents, including aqueous and non-aqueous solvents, dispersion media, coatings, antibacterial and/or antifungal agents, isotonic and/or absorption delaying agents, and/or the like. The use of such media and/or agents for pharmaceutically active substances is well known in the art. Except insofar as any conventional medium, carrier, or agent is incompatible with the active ingredient or ingredients, its use in a composition according to the invention is contemplated. Supplementary active ingredients can also be incorporated into the compositions, particularly as described above.

For administration intranasally or by inhalation, the compounds for use according to the invention are conveniently delivered in the form of an aerosol spray presentation from pressurized packs or a nebulizer, with the use of a suitable propellant, e.g., dichlorodifluoromethane, trichlorofluoromethane, dichlorotetrafluoroethane, carbon dioxide or other suitable gas. In the case of a pressurized aerosol, the dosage unit may be determined by providing a valve to deliver a metered amount. Capsules and cartridges of gelatin for use in an inhaler or insufflator and the like may be formulated containing a powder mix of the compound and a suitable powder base such as lactose or starch.

The compounds may be formulated for parenteral administration by injection, e.g., by bolus injection or continuous infusion. Formulations for injection may be presented in unit-dosage form, e.g., in ampoules or in multi-dose containers, with an added preservative. The compositions may take such forms as suspensions, solutions or emulsions in oily or aqueous vehicles, and may contain formulatory agents such as suspending, stabilizing and/or dispersing agents.

Pharmaceutical formulations for parenteral administration include aqueous solutions of the active compounds in water-soluble form. Additionally, suspensions of the active agents may be prepared as appropriate oily injection suspensions. Suitable lipophilic solvents or vehicles include fatty oils such as sesame oil, or synthetic fatty acid esters, such as ethyl oleate or triglycerides, or liposomes. Aqueous injection suspensions may contain substances that increase the viscosity of the suspension, such as sodium carboxymethyl cellulose, sorbitol, or dextran. Optionally, the suspension may also contain suitable stabilizers or agents, which increase the solubility of the compounds to allow for the preparation of highly concentrated solutions.

Alternatively, the active ingredient may be in powder form for constitution with a suitable vehicle, e.g., sterile pyrogen-free water, before use. The compounds may also be formulated in rectal compositions such as suppositories or retention enemas, e.g., containing conventional suppository bases such as cocoa butter or other glycerides.

In addition to the formulations described above, the compounds may also be formulated as a depot preparation. Such long-acting formulations may be administered by implantation (for example, subcutaneously or intramuscularly) or by intramuscular injection. Thus, for example, the compounds may be formulated with suitable polymeric or hydrophobic materials (for example, as an emulsion in an acceptable oil) or ion-exchange resins, or as sparingly soluble derivatives, for example, as a sparingly soluble salt.

An exemplary pharmaceutical carrier for hydrophobic compounds is a cosolvent system comprising benzyl alcohol, a nonpolar surfactant, a water-miscible organic polymer, and an aqueous phase. The cosolvent system may be a VPD co-solvent system. VPD is a solution of 3% w/v benzyl alcohol, 8% w/v of the nonpolar surfactant polysorbate 80, and 65% w/v polyethylene glycol 300, made up to volume in absolute ethanol. The VPD co-solvent system (VPD:5W) contains VPD diluted 1:1 with a 5% dextrose in water solution. This co-solvent system dissolves hydrophobic compounds well, and itself produces low toxicity upon systemic administration. Naturally, the proportions of a co-solvent system may be varied considerably without destroying its solubility and toxicity characteristics. Furthermore, the identity of the co-solvent components may be varied: for example, other low-toxicity nonpolar surfactants may be used instead of polysorbate 80; the fraction size of polyethylene glycol may be varied; other biocompatible polymers may replace polyethylene glycol, e.g. polyvinyl pyrrolidone; and other sugars or polysaccharides may be substituted for dextrose.

Alternatively, other delivery systems for hydrophobic pharmaceutical compounds may be employed. Liposomes and emulsions are known examples of delivery vehicles or carriers for hydrophobic drugs. Certain organic solvents such as dimethylsulfoxide also may be employed, although usually at the cost of greater toxicity. Additionally, the compounds may be delivered using a sustained-release system, such as semipermeable matrices of solid hydrophobic polymers containing the therapeutic agent. Various sustained-release materials have been established and are known by those skilled in the art. Sustained-release capsules may, depending on their chemical nature, release the compounds for a few weeks up to over 100 days; in other alternatives, depending on the therapeutic agent and the formulation employed, release may occur over hours, days, weeks, or months. Depending on the chemical nature and the biological stability of the therapeutic reagent, additional strategies for protein stabilization may be employed.

The pharmaceutical compositions also may comprise suitable solid- or gel-phase carriers or excipients. Examples of such carriers or excipients include calcium carbonate, calcium phosphate, sugars, starches, cellulose derivatives, gelatin, and polymers such as polyethylene glycols.

A pharmaceutical composition can be administered by a variety of methods known in the art. The routes and/or modes of administration vary depending upon the desired results. Depending on the route of administration, the pharmacologically active agent may be coated in a material to protect the targeting composition or other therapeutic agent from the action of acids and other compounds that may inactivate the agent. Conventional pharmaceutical practice can be employed to provide suitable formulations or compositions for the administration of such pharmaceutical compositions to subjects. Any appropriate route of administration can be employed, for example, but not limited to, intravenous, parenteral, intraperitoneal, intravenous, transcutaneous, subcutaneous, intramuscular, intraurethral, or oral administration. Depending on the severity of the malignancy or other disease, disorder, or condition to be treated, as well as other conditions affecting the subject to be treated, either systemic or localized delivery of the pharmaceutical composition can be used in the course of treatment. The pharmaceutical composition as described above can be administered together with additional therapeutic agents intended to treat a particular disease or condition, which may be the same disease or condition that the pharmaceutical composition is intended to treat, which may be a related disease or condition, or which even may be an unrelated disease or condition.

Pharmaceutical compositions according to the invention can be prepared in accordance with methods well known and routinely practiced in the art. See, e.g., Remington: The Science and Practice of Pharmacy, Mack Publishing Co., 20.sup.th ed., 2000; and Sustained and Controlled Release Drug Delivery Systems, J. R. Robinson, ed., Marcel Dekker, Inc., New York, 1978. Pharmaceutical compositions are typically manufactured under GMP conditions. Formulations for parenteral administration may, for example, contain excipients, sterile water, or saline, polyalkylene glycols such as polyethylene glycol, oils of vegetable origin, or hydrogenated naphthalenes. Biocompatible, biodegradable lactide polymers, lactide/glycolide copolymers, or polyoxyethylene-polyoxypropylene copolymers may be used to control the release of the compounds. Other potentially useful parenteral delivery systems for molecules of the invention include ethylene-vinyl acetate copolymer particles, osmotic pumps, and implantable infusion systems. Formulations for inhalation may contain excipients, for example, lactose, or may be aqueous solutions containing, e.g., polyoxyethylene-9-lauryl ether, glycocholate and deoxycholate, or can be oily solutions for administration or gels.

Pharmaceutical compositions in aspects of the invention are typically administered to the subjects on multiple occasions. Intervals between single dosages can be weekly, monthly or yearly. Intervals can also be irregular as indicated by therapeutic response or other parameters well known in the art. Alternatively, the pharmaceutical composition can be administered as a sustained release formulation, in which case less frequent administration is required. Dosage and frequency vary depending on the half-life in the subject of the pharmacologically active agent included in a pharmaceutical composition. The dosage and frequency of administration can vary depending on whether the treatment is prophylactic or therapeutic. In prophylactic applications, a relatively low dosage is administered at relatively infrequent intervals over a long period of time. Some subjects may continue to receive treatment for the rest of their lives. In therapeutic applications, a relatively high dosage at relatively short intervals is sometimes required until progression of the disease is reduced or terminated, and typically until the subject shows partial or complete amelioration of symptoms of disease. Thereafter, the subject can be administered a prophylactic regime.

For the purposes of the present application, treatment can be monitored by observing one or more of the improving symptoms associated with the disease, disorder, or condition being treated, or by observing one or more of the improving clinical parameters associated with the disease, disorder, or condition being treated. In the case of glioblastoma multiforme and medulloblastoma, the clinical parameters can include, but are not limited to, reduction in tumor burden, reduction in pain, reduction in edema of the brain, reduction in frequency or severity of seizures, reduction in frequency or severity of vomiting, reduction of frequency or severity of headache, reduction in memory deficit, reduction in neurological deficit, and reduction in occurrence of tumor spread or metastasis. As used herein, the terms “treatment,” “treating,” or equivalent terminology are not intended to imply a permanent cure for the disease, disorder, or condition being treated. Compositions and methods according to the invention are not limited to treatment of humans but are applicable to treatment of socially or economically important animals, such as dogs, cats, horses, cows, sheep, goats, pigs, and other animal species of social or economic importance. Unless specifically stated, compositions and methods according to the invention are not limited to the treatment of humans.

Sustained-release formulations or controlled-release formulations are well-known in the art. For example, the sustained-release or controlled-release formulation can be (1) an oral matrix sustained-release or controlled-release formulation; (2) an oral multilayered sustained-release or controlled-release tablet formulation; (3) an oral multiparticulate sustained-release or controlled-release formulation; (4) an oral osmotic sustained-release or controlled-release formulation; (5) an oral chewable sustained-release or controlled-release formulation; or (6) a dermal sustained-release or controlled-release patch formulation.

The pharmacokinetic principles of controlled drug delivery are described, for example, in B. M. Silber et al., “Pharmacokinetic/Pharmacodynamic Basis of Controlled Drug Delivery” in Controlled Drug Delivery: Fundamentals and Applications (J. R. Robinson & V. H. L. Lee, eds, 2d ed., Marcel Dekker, New York, 1987), ch. 5, pp. 213-251. One of ordinary skill in the art can readily prepare formulations for controlled release or sustained release comprising a pharmacologically active agent according to the invention by modifying the formulations described above, such as according to principles disclosed in V. H. K. Li et al, “Influence of Drug Properties and Routes of Drug Administration on the Design of Sustained and Controlled Release Systems” in Controlled Drug Delivery: Fundamentals and Applications (J. R. Robinson & V. H. L. Lee, eds, 2d ed., Marcel Dekker, New York, 1987), ch. 1, pp. 3-94. This process of preparation typically considers physicochemical properties of the pharmacologically active agent, such as aqueous solubility, partition coefficient, molecular size, stability, and nonspecific binding to proteins and other biological macromolecules. This process of preparation also takes into account biological factors, such as absorption, distribution, metabolism, duration of action, the possible existence of side effects, and margin of safety, for the pharmacologically active agent. Accordingly, one of ordinary skill in the art could modify the formulations into a formulation having the desirable properties described above for a particular application.

U.S. Pat. No. 6,573,292 by Nardella, U.S. Pat. No. 6,921,722 by Nardella, U.S. Pat. No. 7,314,886 by Chao et al., and U.S. Pat. No. 7,446,122 by Chao et al., which disclose methods of use of various pharmacologically active agents and pharmaceutical compositions in treating a number of diseases and conditions, including cancer, and methods of determining the therapeutic effectiveness of such pharmacologically active agents and pharmaceutical compositions.

### EXAMPLES

### Mouse Study:

Mice were dosed daily with either:
a. the LXR agonist GW3965 orally at specified concentrations for 5-days,
b. the LXR inverse agonist SR9238 intraperitoneally at specified concentrations for 5-days, or
c. both drugs simultaneously at specified concentrations for 5-days.

Four hours after the final treatment on day 5, mice were euthanized and tissues were collected. Expression of the LXR target gene Abca1 was characterized in the brain along with liver triglycerides to ascertain if LXR activation was achieved in the brain at concentrations absent hepatic triglyceride increases.

TABLE 1
Results

| | GW-3965 | | | | SR9238 | | |
|---|---|---|---|---|---|---|---|
| Dose | — | 15 | 30 | 60 | — | 30 | 60 |
| (mg/kg) | (veh) | | | | (veh) | | |
| Average ABCA1 | 1.0 | 1.6 | 2.0 | 2.8 | 1.0 | 0.9 | 1.2 |
| FC vs. vehicle | | | | | | | |
| (brain) | | | | | | | |
| Average ABCA1 | n/a | *** | **** | **** | n/a | 0.5514 | 0.1929 |
| FC vs. vehicle | | | | | | | |
| (p-value) | | | | | | | |
| Average liver | 9.74 | 16.3 | 18.4 | 29.2 | 8.05 | 7.78 | 8.4 |
| trig (mg/g) | | | | | | | |
| Average liver | n/a | 0.59 | 0.15 | **** | n/a | >0.9999 | >0.9999 |
| trig vs. vehicle | | | | | | | |
| (p-value) | | | | | | | |

Note:
Ordinary one-way ANOVA with Holm-\check{S}ídák's test,
* p < 0.05,
** p < 0.01,
*** p < 0.001,
**** p < 0.0001; 4 hours post dose on day 5,

| | GW-3965 + SR9238 | | | | |
|---|---|---|---|---|---|
| Dose (mg/kg) | — | 30/30 | 30/60 | 60/30 | 60/60 |
| GW-3965/SR9238 | (veh) | | | | |
| Average ABCA1 | 1.0 | 3.6 | 3.4 | 4.2 | 4.3 |
| FC vs. vehicle | | | | | |
| (brain) | | | | | |
| Average ABCA1 | n/a | **** | **** | **** | **** |
| FC vs. vehicle | | | | | |
| (p-value) | | | | | |
| Average liver | 9.79 | 19.9 | 11.4 | 30.9 | 14.3 |
| trig (mg/g) | | | | | |
| Average liver | n/a | * | >0.9999 | **** | 0.99 |
| trig vs. vehicle | | | | | |
| (p-value) | | | | | |

Note:
Ordinary one-way ANOVA with Holm-\check{S}ídák's test,
* p < 0.05,
** p < 0.01,
*** p < 0.001,
**** p < 0.0001; 4 hours post dose on day 5,

Table 1 shows that after 5 days of daily treatment with either an LXR agonist (GW-3965), an LXR inverse agonist (SR9238), or a combination of both, expression of Abca1 in the brains of mice was measured along with hepatic triglyceride concentrations. GW-3965 treatment increased expression of the LXR target gene Abca1 along with increased liver triglyceride concentrations. This GW-3965-mediated elevation of hepatic triglycerides was ablated by co-administration of high dose SR9238. Despite the ablation of hepatic triglyceride levels in mice co-administered GW-3965 and SR9238, Abca1 gene expression was robustly increased in the brains of mice at all dose concentrations.

All references listed herein are individually incorporated herein in their entirety by reference.

Numerous modifications and variations of the invention are possible considering the above teachings. It is therefore to be understood that within the scope of the appended claims, the invention may be practiced otherwise than as specifically described herein.

1. A method of combination therapy, comprising:
(a) administering, to a patient in need thereof, a therapeutically effective amount of an Agonist selected from an LXR Agonist and an RXR Agonist; and,
(b) administering a therapeutically effective amount of an Inhibitor compound that ameliorates one or more side effects associated with the use of the Agonist;
wherein the patient suffers from a disease that is ameliorated by activation of the selected Agonist receptors.

2. The method of claim 1, wherein the LXR Agonist meets the following criteria:
(a) LXRb RLB Ki <100 nM;
(b) LXR Reporter assay EC50 <300 nM; and,
(c) Free drug exposure Cmax in mouse liver >in vitro EC50.

3. The method of claim 1, wherein the RXR Agonist meets the following criteria:
(a) RXR Ki <100 nM;
(b) RXR Reporter assay EC50 <200 nM; and,
(c) Free drug exposure Cmax in mouse liver >in vitro EC50.

4. The method of claim 1, wherein the Agonist is selected from: 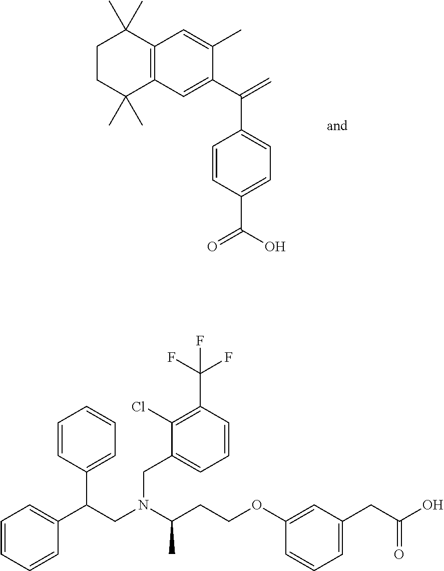 [SMILES: C=C(c1ccc(C(=O)O)cc1)c1cc2c(cc1C)C(C)(C)CCC2(C)C ; C[C@H](CCOc1cccc(CC(=O)O)c1)N(Cc1cccc(C(F)(F)F)c1Cl)CC(c1ccccc1)c1ccccc1]
or a pharmaceutically acceptable salt thereof.

5. The method of claim 1, wherein the Agonist is: 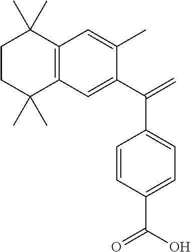 [SMILES: C=C(c1ccc(C(=O)O)cc1)c1cc2c(cc1C)C(C)(C)CCC2(C)C]
or a pharmaceutically acceptable salt thereof.

6. The method of claim 1, wherein the Agonist is: 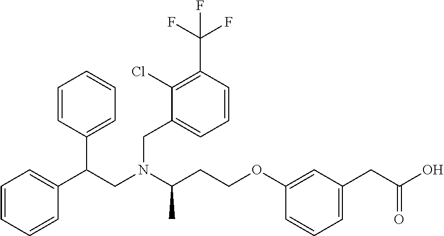 [SMILES: C[C@H](CCOc1cccc(CC(=O)O)c1)N(Cc1cccc(C(F)(F)F)c1Cl)CC(c1ccccc1)c1ccccc1]
or a pharmaceutically acceptable salt thereof.

7. The method of one claim 1, wherein the Inhibitor compound is selected from:
i) an LXR Antagonist,
ii) an LXR Inverse Agonist, and,
iii) a combination thereof.

8. The method of claim 7, wherein the Inhibitor is an LXR Inverse Agonist.

9. The method of claim 1, wherein the LXR Inverse Agonist meets the following criteria:
(a) LXRb RLB Ki <100 nM;
(b) LXR Antagonist Reporter assay EC50 <300 nM; and,
(a) Free drug exposure Cmax in mouse liver >in vitro EC50.

10. The method of claim 8, wherein the LXR Inverse Agonist is selected from: 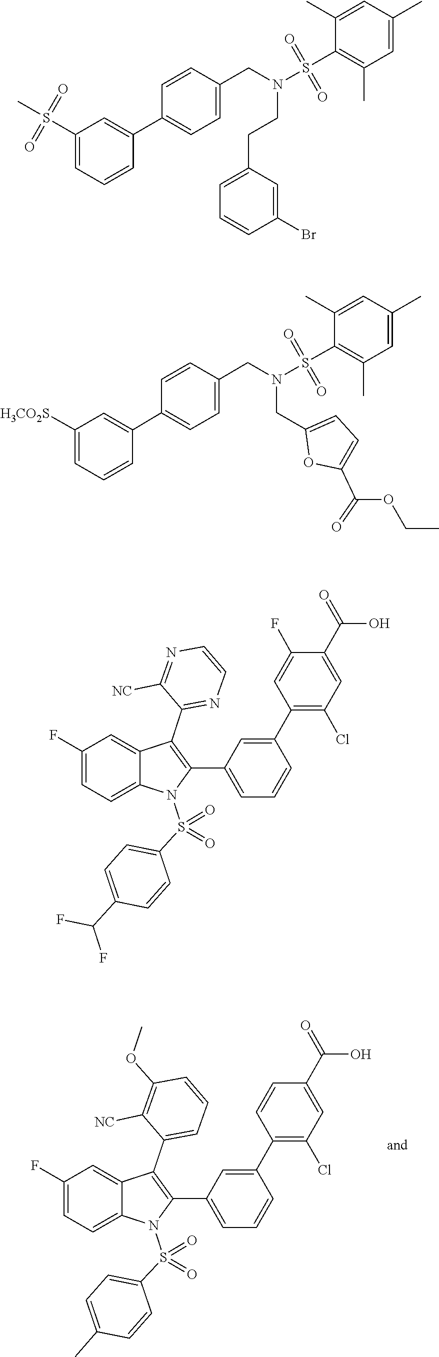 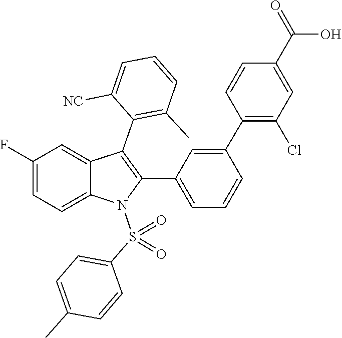 [SMILES: Cc1ccc(S(=O)(=O)n2c(-c3cccc(-c4ccc(C(=O)O)cc4Cl)c3)c(-c3c(C)cccc3C#N)c3cc(F)ccc32)cc1]
or a pharmaceutically acceptable salt thereof.

11. The method of claim 4, wherein the Inhibitor is an LXR Inverse Agonist selected from: 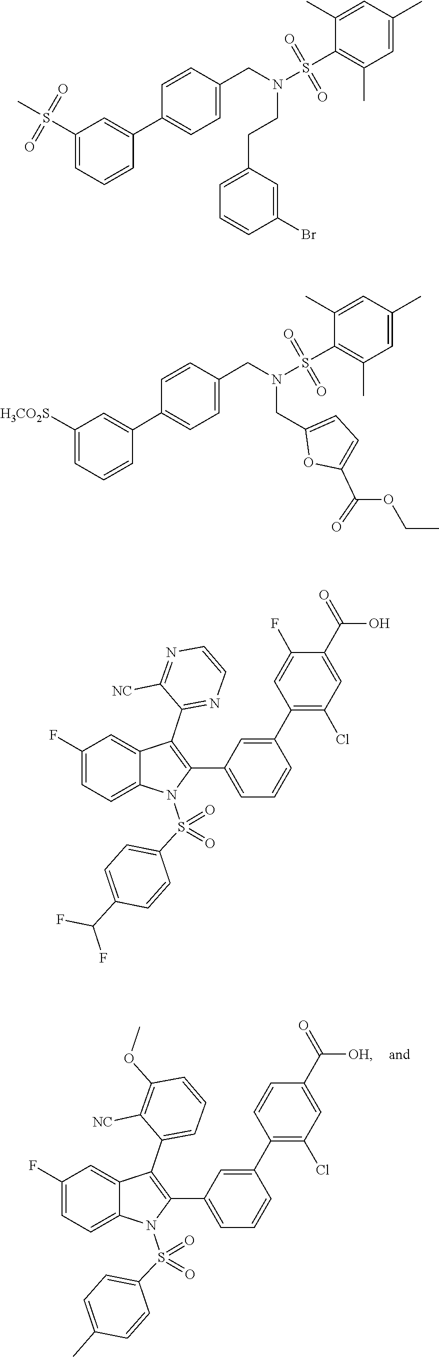 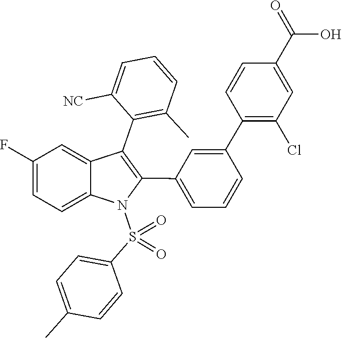 [SMILES: Cc1ccc(S(=O)(=O)n2c(-c3cccc(-c4ccc(C(=O)O)cc4Cl)c3)c(-c3c(C)cccc3C#N)c3cc(F)ccc32)cc1]
or a pharmaceutically acceptable salt thereof.

12. The method of claim 5, wherein the Inhibitor is an LXR Inverse Agonist selected from: 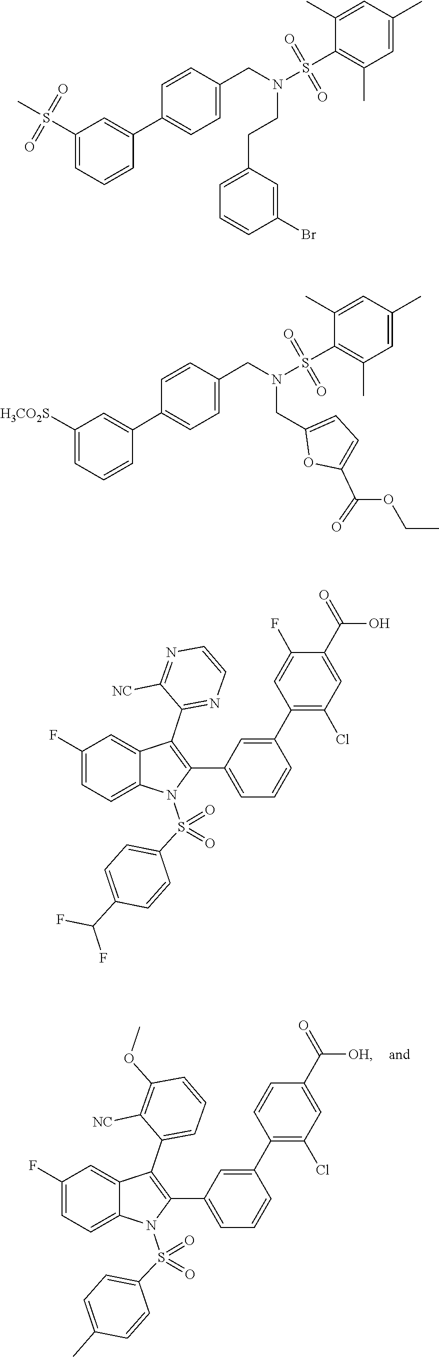 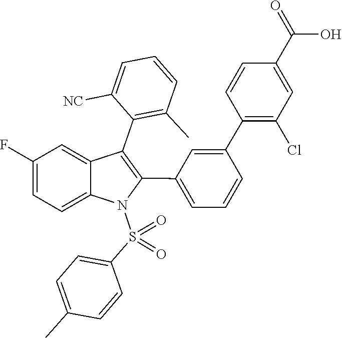 [SMILES: Cc1ccc(S(=O)(=O)n2c(-c3cccc(-c4ccc(C(=O)O)cc4Cl)c3)c(-c3c(C)cccc3C#N)c3cc(F)ccc32)cc1]
or a pharmaceutically acceptable salt thereof.

13. The method of claim 5, wherein the Inhibitor is an LXR Inverse Agonist of the formula: 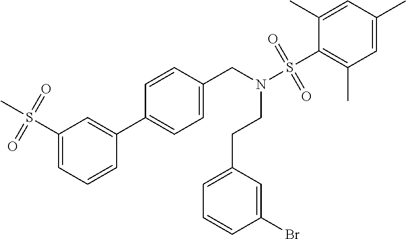
or a pharmaceutically acceptable salt thereof.

14. The method of claim 5, wherein the Inhibitor is an LXR Inverse Agonist of the formula: 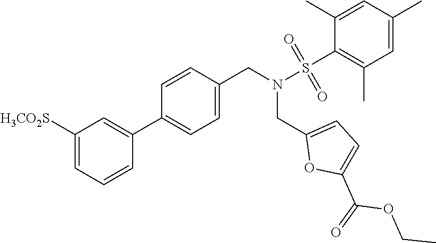
or a pharmaceutically acceptable salt thereof.

15. The method of claim 5, wherein the Inhibitor is an LXR Inverse Agonist of the formula: 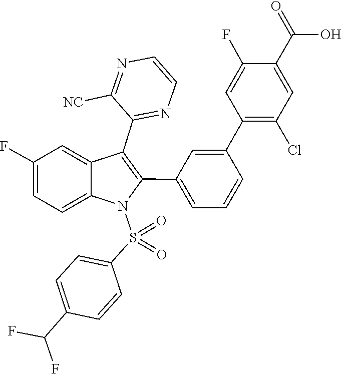 [SMILES: N#Cc1nccnc1-c1c(-c2cccc(-c3cc(F)c(C(=O)O)cc3Cl)c2)n(S(=O)(=O)c2ccc(C(F)F)cc2)c2ccc(F)cc12]
or a pharmaceutically acceptable salt thereof.

16. The method of claim 5, wherein the Inhibitor is an LXR Inverse Agonist of the formula: 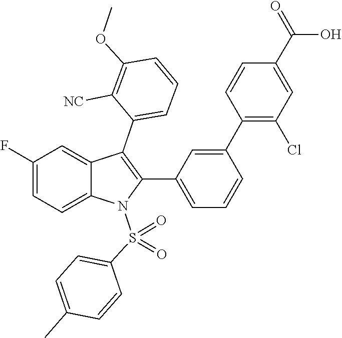 [SMILES: COc1cccc(-c2c(-c3cccc(-c4ccc(C(=O)O)cc4Cl)c3)n(S(=O)(=O)c3ccc(C)cc3)c3ccc(F)cc23)c1C#N]
or a pharmaceutically acceptable salt thereof.

17. The method of claim 5, wherein the Inhibitor is an LXR Inverse Agonist of the formula: 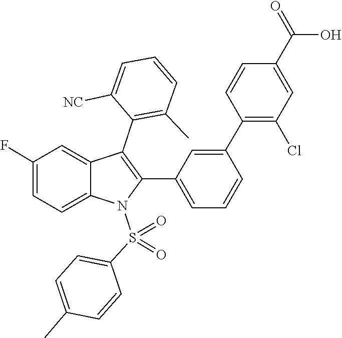 [SMILES: Cc1ccc(S(=O)(=O)n2c(-c3cccc(-c4ccc(C(=O)O)cc4Cl)c3)c(-c3c(C)cccc3C#N)c3cc(F)ccc32)cc1]
or a pharmaceutically acceptable salt thereof.

18. The method of claim 6, wherein the Inhibitor is an LXR Inverse Agonist selected from: 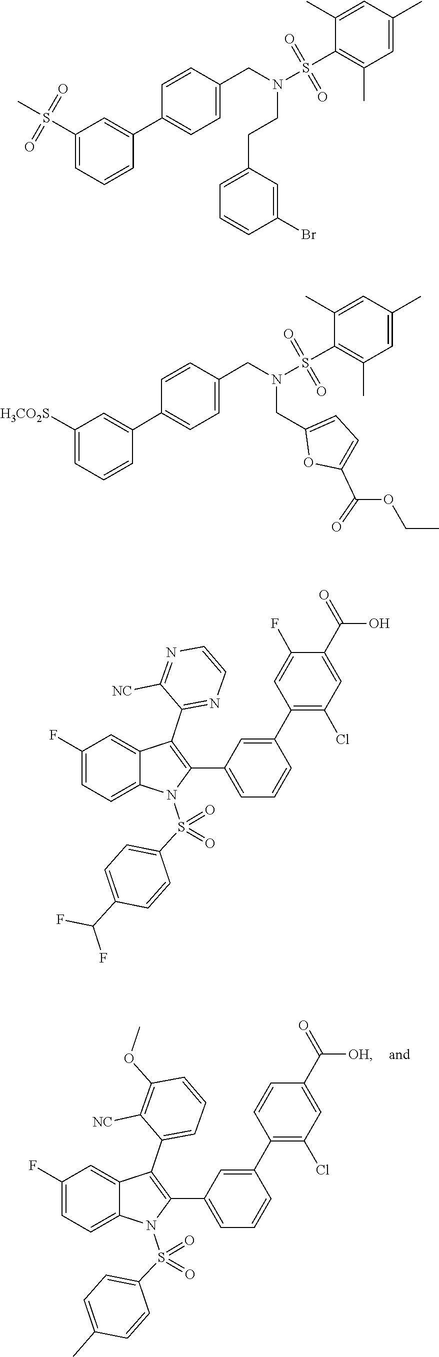 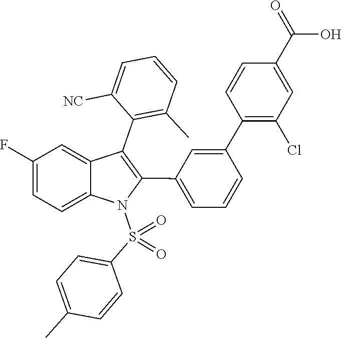 [SMILES: Cc1ccc(S(=O)(=O)n2c(-c3cccc(-c4ccc(C(=O)O)cc4Cl)c3)c(-c3c(C)cccc3C#N)c3cc(F)ccc32)cc1]
or a pharmaceutically acceptable salt thereof.

19. The method of claim 6, wherein the Inhibitor is an LXR Inverse Agonist of the formula: 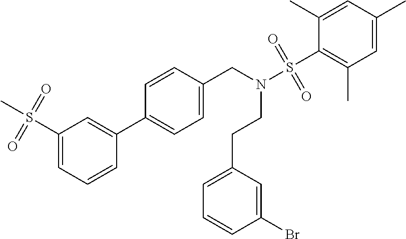
or a pharmaceutically acceptable salt thereof.

20. The method of claim 6, wherein the Inhibitor is an LXR Inverse Agonist of the formula: 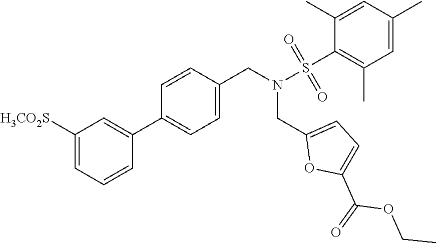
or a pharmaceutically acceptable salt thereof.

21. The method of claim 6, wherein the Inhibitor is an LXR Inverse Agonist of the formula: 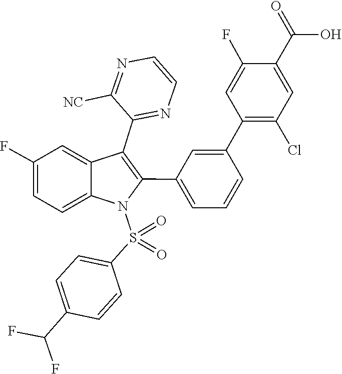 [SMILES: N#Cc1nccnc1-c1c(-c2cccc(-c3cc(F)c(C(=O)O)cc3Cl)c2)n(S(=O)(=O)c2ccc(C(F)F)cc2)c2ccc(F)cc12]
or a pharmaceutically acceptable salt thereof.

22. The method of claim 6, wherein the Inhibitor is an LXR Inverse Agonist of the formula: 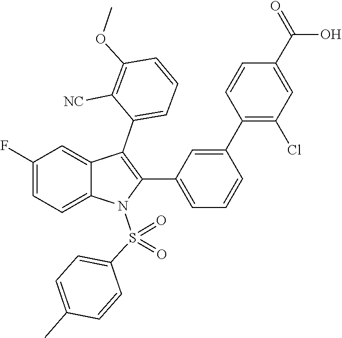 [SMILES: COc1cccc(-c2c(-c3cccc(-c4ccc(C(=O)O)cc4Cl)c3)n(S(=O)(=O)c3ccc(C)cc3)c3ccc(F)cc23)c1C#N]
or a pharmaceutically acceptable salt thereof.

23. The method of claim 6, wherein the Inhibitor is an LXR Inverse Agonist of the formula: 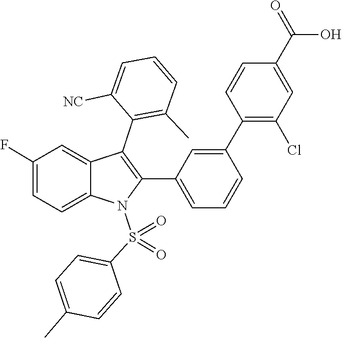 [SMILES: Cc1ccc(S(=O)(=O)n2c(-c3cccc(-c4ccc(C(=O)O)cc4Cl)c3)c(-c3c(C)cccc3C#N)c3cc(F)ccc32)cc1]
or a pharmaceutically acceptable salt thereof.

24. The method of claim 1, wherein the Agonist is an LXR Agonist and the patient suffers from a disease selected from: cancer, a neurodegenerative disease, and atherosclerosis.

25. The method of claim 1, wherein the Agonist is an RXR Agonist and the patient suffers from a disease selected from: cutaneous T-cell lymphoma, a neurodegenerative disease, immune-oncology, and an inflammatory disease.

26. A pharmaceutical composition, comprising:
(a) one or more LXR Agonists or RXR Agonists;
(b) one or more Inhibitor compounds; and,
(c) a pharmaceutically acceptable carrier.

27. The composition of claim 24, wherein the Agonist is selected from: 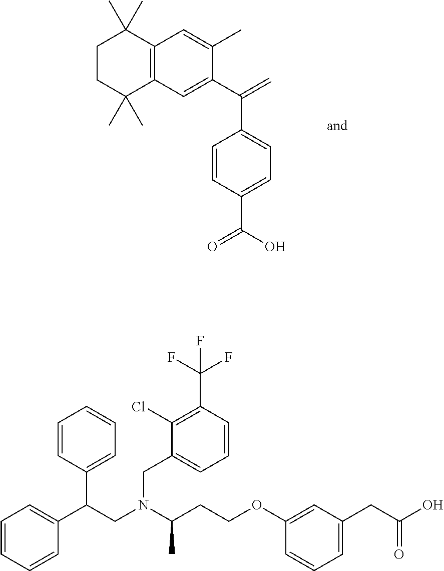 [SMILES: C=C(c1ccc(C(=O)O)cc1)c1cc2c(cc1C)C(C)(C)CCC2(C)C ; C[C@H](CCOc1cccc(CC(=O)O)c1)N(Cc1cccc(C(F)(F)F)c1Cl)CC(c1ccccc1)c1ccccc1]
or a pharmaceutically acceptable salt thereof.

28. The composition of claim 24, wherein the Inhibitor compound is an LXR Inverse Agonist.

29. The composition of claim 27, wherein the LXR Inverse Agonist is selected from: 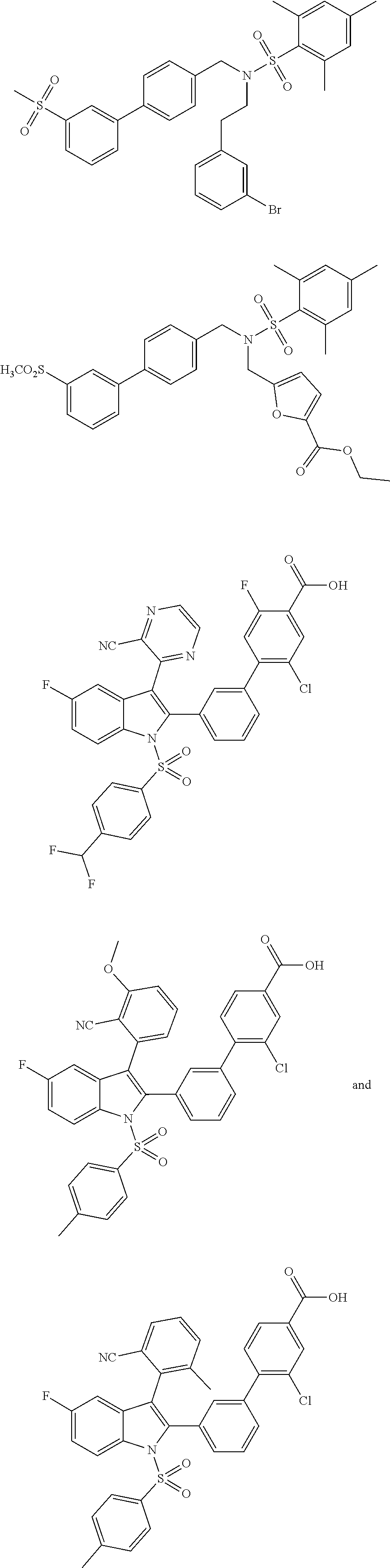
or a pharmaceutically acceptable salt thereof.
